(19) United States
(12) Patent Application Publication
(10) Pub. No.: US 2021/0020294 A1
(43) Pub. Date: Jan. 21, 2021

(54) METHODS, DEVICES AND SYSTEMS FOR HOLISTIC INTEGRATED HEALTHCARE PATIENT MANAGEMENT

(71) Applicant: Pacesetter, Inc., Sylmar, CA (US)

(72) Inventors: Rupinder Bharmi, Canyon Country, CA (US); Rahul Agarwal, Los Angeles, CA (US); Philip B. Adamson, Austin, TX (US); Heidi Hellman, Atlanta, GA (US); Nirav Dalal, Porter Ranch, CA (US)

(21) Appl. No.: 16/930,791

(22) Filed: Jul. 16, 2020

Related U.S. Application Data
(60) Provisional application No. 62/875,870, filed on Jul. 18, 2019.

(51) Int. Cl. G16H 20/30; G16H 50/20; G16H 20/17

(52) U.S. Cl. CPC G16H 20/30; G16H 20/17; G16H 50/20

(57) ABSTRACT

A method for managing a treatment is provided. The method is under control of a processor. The method obtains a body generated analyte (BGA) indicative of a malnutrition state (MS) characteristic of interest (COI) of a patient and obtains implantable medical device (IMD) data indicative of a physiologic COI from the patient. The method assigns a health risk index based on the MS COI and the physiologic COI. The health risk index is indicative of a chronic disease state and malnutrition state currently exhibited by the patient. The method generates a treatment notification based on the health risk index.

### REFERENCE TO RELATED APPLICATIONS

The present application claims priority to U.S. Provisional Application No: 62/875,870, Titled “METHODS, DEVICE AND SYSTEMS FOR HOLISTIC INTEGRATED HEALTHCARE PATIENT MANAGEMENT” which was filed on Jul. 18, 2019, the complete subject matter of which is expressly incorporated herein by reference in its entirety.

### BACKGROUND

Embodiments of the present disclosure generally relate to methods, devices and systems for holistic management of patients with chronic disease based on an integrated analysis of dissimilar patient data from unrelated data sources.

A variety of technologies and solutions are offered today for managing patients with chronic diseases. For example, certain solutions focus on patient nutrition, while other solutions utilize lab diagnostics to monitor certain characteristics of interest from blood samples or other bodily fluid samples. In addition, passive implantable medical devices are provided to monitor pulmonary arterial pressure, while other implantable medical devices are provided to monitor other diagnostics. As another example of passive implantable medical devices, structural heart procedures are provided (e.g., transcatheter valve solutions, surgical valvular solutions, structural heart occluders) to advanced heart failure therapy. As a further example, active implantable medical devices are provided, such as pacemakers, defibrillators, cardiac resynchronization therapy devices and the like.

However, the various technologies and solutions are provided somewhat independent of one another and without full regard for patient information available from all other data sources. Often, clinical management decisions are made based on fragments of patient information that the clinician has at the time of the decision. The clinician must manually piece together patient data each time the clinician renders a patient management decision.

A need remains for an integrated approach, that considers patient data from dis-similar sources, when managing patients with chronic disease. Further, a need remains for systems and methods that support periodic measurements of patient vital signs and disease specific analytes, as opposed to need-based/clinical visit-based measurements

### SUMMARY

In accordance with embodiments herein, methods, systems and devices are provided that integrate external diagnostic information (e.g., laboratory or point of care diagnostic information) with streaming data from implantable medical devices in order to provide a holistic patient management environment. In accordance with embodiments herein, methods, systems and devices are provided for applications of the holistic patient management environment, in which meaningful clinical insights are derived from relevant sources of information. The holistic patient management environment allows for optimal management of the patient as it relates to specific one-to-one care received at the clinic or hospital, and also allow for remote observation of the patient to allow for longitudinal management of the patient.

In order to provide holistic patient management, the methods, systems and devices integrate patient data from dissimilar data sources into a healthcare system that remotely monitors implantable and external medical and test devices. As nonlimiting examples, the holistic patient management environment utilizes IMD data and BGA data from dis-similar sources, in at least the following applications:

1. Providing early diagnosis and treatment recommendations for patients who experience chronic disease and malnutrition;

2. Managing treatment for heart failure patients;

3. Managing prediction of hospitalizations for heart failure patients;
4. Managing detection of myocardial ischemia; and
5. Managing an INR level for patients with mechanical circulatory support devices.

In accordance with embodiments herein, methods, systems and devices are provided that incorporate digital healthcare management and patient application features, as implemented on handheld devices, to leverage information known about various food products and a patient's diet. For example, smart phone-based applications make it simple to track calorie, salt and fat intake, and to combine such information with activity collected by an IMD, wearable device and the like. The information is analyzed to identify a treatment diagnosis and recommendation. Among other things, the recommendation may include a communication to a patient and/or caregiver that includes educational material and/or feedback regarding negative/positive health consequences of the patient's lifestyle choices. The recommendation may inform a patient of trends in certain physiologic characteristics of interest and provide feedback and/or encouragement when patient data improves or indicates a positive trend. From a clinician perspective, the insights from the trends from a patient and their treatment would provide feedback on the suitability of the treatment for the patient (e.g., what is working, what is not working for the patients).

In accordance with embodiments herein, a method and system are provided for managing a treatment. The method and system are under control of a processor. The method and system obtain a body generated analyte (BGA) indicative of a malnutrition state (MS) characteristic of interest (COI) of a patient and obtains implantable medical device (IMD) data indicative of a physiologic COI from the patient. The method and system assign a health risk index based on the MS COI and the physiologic COI. The health risk index is indicative of a chronic disease state and malnutrition state currently exhibited by the patient. The method and system generate a treatment notification based on the health risk index.

Optionally, the method and system may calculate a malnutrition state related index (MSI) based on the BGA data. The MSI may be indicative of a degree of malnutrition experienced by the patient. The method and system may calculate a congestion state related index (CSI) based on the IMD data, the CSI indicative of a degree of congestion experienced by the patient. The generating the treatment notification may include generating a diagnosis and treatment recommendation based on the MSI and CSI. The treatment recommendation may include changing at least one of a medication or dosage level. The BGA data may include a serum albumin level of the patient the calculating comprises calculating a geriatric nutrition risk index (GNRI) level based on the serum albumin level.

Optionally, the method and system may classify the MSI to have one of a number of malnutrition states based on a correlation between the GNRI level and predetermined ranges. The method and system may calculate a malnutrition state by applying at least one of a geriatric nutrition risk index, a controlling nutrition status index or a prognostic nutrition index to the BGA data. The method and system may apply one or more application specific models (ASM) to calculate the health risk index. The ASM may be implemented as at least one of a threshold-based algorithm, template correlation algorithm, lookup table, decision tree, or machine learning algorithm.

In accordance with embodiments herein, a method and system are provided for managing treatment for a heart failure (HF) patient. The method and system are under control of a processor. The method and system obtain electrolyte related body generated analyte (BGA) data indicative of a level of an electrolyte characteristic of interest (COI) of a patient, obtains diuretic medication information indicative of a diuretic prescription to a patient and obtains implantable medical device (IMD) data that includes hemodynamic data indicative of a hemodynamic COI experienced by the patient following the diuretic prescription. The method and system determine a diuretic response profile of the patient based on the diuretic medication information and hemodynamic data and generates an HF diagnosis based on the diuretic response profile. The level of the electrolyte COI indicated by the electrolyte related BGA data. The method and system generate an HF treatment notification based on the HF diagnosis.

Optionally, the method and system may calculate a level for at least one of glomerular filtration rate (GFR), blood urea nitrogen (BUN) or creatinine from the electrolyte related BGA data. The calculating operation may include calculating the level for the GFR. The HF diagnosis may include recommending an increase or decrease in the diuretic prescription based on predetermined combinations of changes in the hemodynamic data and the level of the GFR. The method and system may identify cardiorenal syndrome (CRS) based on an increase in the BUN and creatinine. The method and system may assign a health risk index of advanced AF, when the CRS is identified in combination with a downward trend in cardiac output and a diuretic resistance indicated by the diuretic response profile. The BGA data may include malnutrition state BGA (MS-BGA) data indicative of a malnutrition state (MS) COI of the patient. The HF diagnosis may declare one of an intravascular volume overload or a total body overload based on the hemodynamic data, diuretic response profile and MS-BGA data.

Optionally, the hemodynamic data may include pulmonary arterial pressure (PAP) data indicative of a pulmonary arterial pressure experienced by the patient following the diuretic prescription. The diuretic response profile may be determined based on the PAP data. The electrolyte related BGA data may include a glucose level indicative of a blood sugar level for the patient. The method and system may further comprise identifying episodes of increased PAP associated with a decrease in the blood glucose level, and based thereon, may generate the HF diagnosis that avoids an increase in a dosage of the diuretic prescription. The diagnosis may include a recommendation to adjust intake of a nutritional supplement configured to correct a malnutrition state and avoid an increase in a dosage of the diuretic prescription.

In accordance with embodiments herein, a method and system are provided for prioritizing patients for clinician management. The method and system are under control of a processor. The method and system obtain related body generated analyte (BGA) data indicative of a characteristic of interest (COI) of a heart. The method and system obtain implantable medical device (IMD) data indicative of a COI experienced by the heart and determines a risk score for a patient based on the BGA data and the IMD data. The risk score is related to a probability that a patient will experience a predetermined event during a predetermined period of time. The method and system generate an HF diagnosis based on the risk score and generates an HF treatment notification based on the HF diagnosis.

Optionally, the method and system may calculate an overall probability that the patient will experience a heart failure episode, as the predetermined event. The determining the risk score may comprise assigning a level to the risk score based on the overall probability. The IMD data may include pulmonary arterial pressure (PAP) data. The determining the risk score may comprise estimating a PAP probability that a patient will experience the predetermined event during the predetermined period of time based on at least one of a PAP level or PAP trend.

Optionally, the BGA data may represent cardiac marker related BGA data that may include B-type natriuretic peptide (BNP) data. The determining the risk score may comprise estimating a BNP probability that the patient will experience the predetermined event during the predetermined period of time based on at least one of a BNP level or BNP trend and may calculate the overall probability based on a combination of the PAP and BNP probabilities.

Optionally, the method and system may compare the priority score of the patient to priority scores of other patients, may assign a rank to the patient based on the comparing operation and may schedule an appointment for the patient based on the rank. The risk score may be related to a cause of hospitalization associated with at least one of kidney failure, nutrition deficiencies, malnutrition and pneumonia.

In accordance with embodiments herein, a method and system are provided for detecting myocardial ischemia (MI). The method and system are under control of a processor. The method and system obtain cardiac enzyme related body generated analyte (BGA) data indicative of a level of a cardiac enzyme characteristic of interest (COI) of a patient and obtains implantable medical device (IMD) data that includes cardiac activity (CA) signals for one or more cardiac beats. The method and system determine an ST segment level for the one or more cardiac beats based on the CA signals. The method and system generate an MI diagnosis based on the ST segment level and a level of the cardiac enzyme COI indicated by the cardiac enzyme related BGA data and generates an MI treatment notification based on the MI diagnosis.

Optionally, the method and system may calculate a troponin level for at least one of troponin I or troponin T from the cardiac enzyme related BGA data. The MI diagnosis generated may be based on the troponin level. The MI diagnosis may include identifying at least one of an acute myocardial ischemia or myocardial infarction. The obtaining the cardiac enzyme related BGA data may include implementing a first test with an at home point-of-care BGA test device to obtain a first troponin level, and implementing, at a later point in time, a second test with a medical facility BGA test device to obtain a second troponin level. The generating the MI diagnosis may be based on a relation between the first and second troponin levels.

Optionally, the obtaining the cardiac enzyme related BGA data may include implementing, while a patient is not experiencing a select myocardial infarction symptom, a first test to obtain a first troponin level, and implementing, while the patient is experiencing the select myocardial infarction symptom, a second test to obtain a second troponin level. The select myocardial infarction symptom may represent chest pain. The determining the ST segment level may further comprise analyzing the ST segment for the one or more cardiac beats to identify a shift to an elevated level with respect to a prior or baseline ST segment level. The MI diagnosis may include identifying at least one of an acute myocardial ischemia or myocardial infarction when the shift in the ST segment level exceeds a threshold.

Optionally, the method and system may automatically identify a baseline ST segment level, the determining operation further comprising comparing the ST segment level to the baseline ST segment level to identify a shift there between. The method and system may obtain the cardiac enzyme related BGA data and may generate the MI diagnosis and may generate the MI treatment notification are performed while a patient is at a medical facility. The obtaining the IMD data may be performed before at a point in time before the patient is at the medical facility. The EMI diagnosis may indicate an acute myocardial ischemia. The EMI diagnosis may be generated at an early stage while a patient is experiencing a symptom of chest pain. The method may obtain heart rate information. The generating the EMI diagnosis may be based in part on the heart rate information.

In accordance with embodiments herein, a method and system are provided for managing treatment of the patient with a mechanical circulation support device. The method and system are under control of a processor. The method and system obtain implantable medical device (IMD) data that includes cardiac activity (CA) signals for one or more cardiac beats and analyzes the CA signals to determine an atrial fibrillation (AF) burden. The method and system obtain body generated analyte (BGA) data and anticoagulant medication information indicative of an anticoagulant prescription to a patient and determines a level for an international normalized ratio (INR) indicative of a blood clotting (BC) characteristic of interest (COI) of a patient based on the BGA data. The method and system generate an INR diagnosis based on the AF burden, the level of the INR and the anticoagulant medication information and generates an INR treatment notification based on the INR diagnosis.

Optionally, the IMD data may include mechanical circulatory support (MCS) data indicative of a parameter of an MCS device. The INR diagnosis may be generated in part based on the parameter of the MCS device. The parameter may be indicative of at least one of an RPM level, flow rate or device alert from the MCS device. The IMD data may include a parameter that is indicative of at least one of an RPM level, flow rate or device alert from a ventricular assist device (VAD). The analyzing may further comprise continuously analyzing the AF burden and the parameter from the VAD in connection with risk of at least one of hemolysis or thrombosis

Optionally, the analyzing the CA signals may include identifying an onset of AF burden after implant of a left ventricular assist device. The anticoagulant prescription may include a warfarin prescription. The generating the INR treatment notification may include an adjustment to the warfarin prescription in connection with targeting the INR to between 2.0 and 3.0. The generating the INR diagnosis may include determining whether to increase, maintain or reduce a dosage of an anticoagulation prescription. In accordance with new and unique aspects herein, a method is provided for managing treatment for a patient, the method comprising: under control of a processor, obtaining a medical data collection, for the patient, that includes at least two of the following a) to e): a) implantable medical device (IMD) data indicative of at least one of ai) a hemodynamic COI experienced by the patient, aii) a physiologic COI from the patient, aiii) cardiac activity (CA) signals for one or more cardiac beats; b) body generated analyte (BGA) data indicative of at least one of bi) a malnutrition state (MS) COI of a patient, bii) electrolyte COI of a patient, biii) a COI of a heart, iv) a cardiac enzyme COI of a patient; c) diuretic medication information indicative of a diuretic prescription to a patient; d) anticoagulant medication information indicative of an anticoagulant prescription to a patient; or e) behavior related medical (BRM) data indicative of an action, conduct or state by a patient in connection with one or more physiologic COI. The method applies an application-specific model (ASM) to the medical data collection to determine a diagnosis and a treatment notification based on the diagnosis, wherein the ASM is implemented as at least one of a threshold-based algorithm, template correlation algorithm, lookup table, decision tree, or machine learning algorithm. The ASM performs at least one of the following a) to f) determining operations to determine the diagnosis and treatment notification: f) determining at least one of: f1) whether a patient is a candidate for a procedure, f2) an effectiveness of a prior procedure, f3) a degree of perfusion from pulmonary hypertension experienced by the patient; g) determining a health risk index indicative of a chronic disease state and malnutrition state currently exhibited by the patient; h) determining h1) a diuretic response profile of the patient based on the diuretic medication information and hemodynamic data, h2) an heart failure (HF) diagnosis based on the diuretic response profile, the level of the electrolyte COI indicated by the BGA data; and h3) an HF treatment notification based on the HF diagnosis; i) determining:) a risk score for a patient based on the BGA data and the IMD data, the risk score related to a probability that a patient will experience a predetermined event during a predetermined period of time; i2) an HF diagnosis based on the risk score; and d3) an HF treatment notification based on the HF diagnosis; j) determining: j1) an ST segment level for the one or more cardiac beats based on the CA signals; j2) an myocardial infarction (MI) diagnosis based on the ST segment level and a level of the cardiac enzyme COI indicated by the BGA data; and j3) an MI treatment notification based on the MI diagnosis; or k) determining: k1) a level for an international normalized ratio (INR) indicative of a blood clotting (BC) characteristic of interest (COI) of a patient based on the BGA data; k2) an INR diagnosis based on the AF burden, the level of the INR and the anticoagulant medication information; and k3) an INR treatment notification based on the INR diagnosis.

In accordance with new and unique aspects herein, the medical data collection includes the IMD data indicative of the hemodynamic COI experienced by the patient and the ASM determines, as the treatment notification, an indication that a patient is or is not a candidate for at least one of implant of a ventricular assist device, a transplant, or a valve repair procedure. In accordance with new and unique aspects herein, the medical data collection includes first and second IMD data from first and second IMDs, respectively, the first IMD including a pulmonary arterial pressure (PAP) sensor, the first IMD data corresponding to hemodynamic data collected by the PAP sensor indicative of ai) the hemodynamic COI, the second IMD connected to two or more subcutaneous electrodes configured to sense CA signals as the second IMD data. Additionally or alternatively, the ASM analyzes the first and second IMD data, to determine whether the patient is experiencing a select degree of perfusion resulting from pulmonary hypertension. In accordance with new and unique aspects herein, the medical data collection includes the IMD data indicative of the hemodynamic COI experienced by the patient, the method further comprising obtaining the IMD data after the patient has undergone a surgical procedure, the treatment notification including an indication regarding an effectiveness of the procedure. In accordance with new and unique aspects herein, the medical data collection comprises the BGA data indicative of the MS COI of the patient and further comprising the IMD data indicative of the physiologic COI from the patient, the ASM determining the health risk index by: calculating a malnutrition state related index (MSI) based on the BGA data, the MSI indicative of a degree of malnutrition experienced by the patient, calculating a congestion state related index (CSI) based on the IMD data, the CSI indicative of a degree of congestion experienced by the patient; and generating the diagnosis and treatment recommendation based on the MSI and CSI.

In accordance with new and unique aspects herein, BGA data includes a serum albumin level of the patient, the ASM calculating a geriatric nutrition risk index (GNRI) level based on the serum albumin level, the ASM classifying the MSI to have one of a number of malnutrition states based on a correlation between the GNRI level and predetermined ranges. In accordance with new and unique aspects herein, the medical data collection comprises the BGA data indicative of the level of the electrolyte COI of the patient, the ASM determining the diagnosis and treatment notification, in part, by calculating a level for at least one of glomerular filtration rate (GFR), blood urea nitrogen (BUN) or creatinine from the BGA data. Additionally or alternatively, the diagnosis represents a HF diagnosis including recommending an increase or decrease in the diuretic prescription based on predetermined combinations of changes in the hemodynamic data and the level of the GFR. Additionally or alternatively, the method further identifies cardiorenal syndrome (CRS) based on an increase in the BUN and creatinine, assigning a health risk index of advanced AF, when the CRS is identified in combination with a downward trend in cardiac output and a diuretic resistance indicated by the diuretic response profile. In accordance with new and unique aspects herein, at least one of: i) the BGA data includes malnutrition state BGA (MS-BGA) data indicative of a malnutrition state (MS) COI of the patient, wherein the HF diagnosis declares one of an intravascular volume overload or a total body overload based on the hemodynamic data, diuretic response profile and MS-BGA data; ii) the BGA data includes a glucose level indicative of a blood sugar level for the patient, the method further comprising identifying episodes of increased pulmonary arterial pressure associated with a decrease in the blood glucose level, and based thereon, generating the HF diagnosis that avoids an increase in a dosage of the diuretic prescription; or iii) the diagnosis includes a recommendation to adjust intake of a nutritional supplement configured to correct a malnutrition state and avoid an increase in a dosage of the diuretic prescription.

In accordance with new and unique aspects herein, a system is provided for managing treatment for a patient, the system comprising: memory configured to store program instructions; and an input configured to obtain a medical data collection, for the patient, that includes at least two of the following a) to e): a) implantable medical device (IMD) data indicative of at least one of ai) a hemodynamic COI experienced by the patient, aii) a physiologic COI from the patient, aiii) cardiac activity (CA) signals for one or more cardiac beats; b) body generated analyte (BGA) data indicative of at least one of bi) a malnutrition state (MS) characteristic of interest (COI) of a patient, bii) an electrolyte COI of a patient, biii) a COI of a heart, iv) a cardiac enzyme COI of a patient; c) diuretic medication information indicative of a diuretic prescription to a patient; d) anticoagulant medication information indicative of an anticoagulant prescription to a patient; or e) behavior related medical (BRM) data indicative of an action, conduct or state by a patient in connection with one or more physiologic COI. The system further includes a processor configured to implement the program instructions to: apply an application-specific model (ASM) to the medical data collection to determine a diagnosis and a treatment notification based on the diagnosis; wherein the ASM is implemented as at least one of a threshold-based algorithm, template correlation algorithm, lookup table, decision tree, or machine learning algorithm. The ASM is configured to perform at least one of the following a) to f) determine operations to determine the diagnosis and treatment notification: f) determine at least one of: f1) whether a patient is a candidate for a procedure, f2) an effectiveness of a prior procedure, f3) a degree of perfusion from pulmonary hypertension experienced by the patient; g) determine a health risk index indicative of a chronic disease state and malnutrition state currently exhibited by the patient; h) determine h1) a diuretic response profile of the patient based on the diuretic medication information and hemodynamic data, h2) an heart failure (HF) diagnosis based on the diuretic response profile, the level of the electrolyte COI indicated by the BGA data; and h3) an HF treatment notification based on the HF diagnosis; i) determine:) a risk score for a patient based on the BGA data and the IMD data, the risk score related to a probability that a patient will experience a predetermined event during a predetermined period of time; i2) an HF diagnosis based on the risk score; and d3) an HF treatment notification based on the HF diagnosis; j) determine: j1) an ST segment level for the one or more cardiac beats based on the CA signals; j2) an myocardial infarction (MI) diagnosis based on the ST segment level and a level of the cardiac enzyme COI indicated by the BGA data; and j3) an MI treatment notification based on the MI diagnosis; or k) determine: k1) a level for an international normalized ratio (INR) indicative of a blood clotting (BC) characteristic of interest (COI) of a patient based on the BGA data; k2) an INR diagnosis based on the AF burden, the level of the INR and the anticoagulant medication information; and k3) an INR treatment notification based on the INR diagnosis.

In accordance with new and unique aspects herein, the medical data collection includes BGA data indicative of the COI of the heart and IMD data indicative of the COI of the heart, the processor is configured to implement the ASM to: determine a risk score for a patient based on the BGA data and the IMD data, the risk score related to a probability that a patient will experience a predetermined event during a predetermined period of time; generate, as the diagnosis, an HF diagnosis based on the risk score; and generate, as the treatment notification, an HF treatment notification based on the HF diagnosis. In accordance with new and unique aspects herein, IMD data includes pulmonary arterial pressure (PAP) data, the processor configured to implement the ASM to at least one of: estimate a PAP probability that the patient will experience the predetermined event during the predetermined period of time based on at least one of a PAP level or PAP trend; or calculate an overall probability that the patient will experience a heart failure episode, as the predetermined event, the determining the risk score comprising assigning a level to the risk score based on the overall probability.

In accordance with new and unique aspects herein, the IMD data includes the CA signals for one or more cardiac beats, the processor configured to implement the ASM to: determine an ST segment level for the one or more cardiac beats based on the CA signals; generate, as the diagnosis, a myocardial infarction (MI) diagnosis based on the ST segment level and a level of the cardiac enzyme COI indicated by the cardiac enzyme related BGA data; and generate, as the treatment notification, an MI treatment notification based on the MI diagnosis.

In accordance with new and unique aspects herein, the processor is further configured to implement the ASM to calculate a troponin level for at least one of troponin I or troponin T from the BGA data, the MI diagnosis generated based on the troponin level. In accordance with new and unique aspects herein, the BGA data includes obtaining cardiac enzyme related BGA data by implementing a first test with an at home point-of-care BGA test device to obtain a first troponin level, and by implementing, at a later point in time, a second test with a medical facility BGA test device to obtain a second troponin level, the generating the MI diagnosis based on a relation between the first and second troponin levels. In accordance with new and unique aspects herein, the medical data collection includes the IMD data indicative of CA signals for one or more cardiac beats, the BGA data and the anticoagulation medication information indicative of the anticoagulant prescription to the patient, the processor configured to implement the ASM to: determine an atrial fibrillation (AF) burden based on the CA signals; determine a level for an international normalized ratio (INR) indicative of a blood clotting (BC) characteristic of interest (COI) of a patient based on the BGA data; generate an INR diagnosis based on the AF burden, the level of the INR and the anticoagulant medication information; and generate an INR treatment notification based on the INR diagnosis.

In accordance with new and unique aspects herein, the IMD data further includes mechanical circulatory support (MCS) data indicative of a parameter of an MCS device, the INR diagnosis generated in part based on the parameter of the MCS device. In accordance with new and unique aspects herein, the IMD data includes a parameter that is indicative of at least one of an RPM level, flow rate or device alert from a ventricular assist device (VAD), the processor further configured to implement the ASM to analyze the AF burden and the parameter from the VAD in connection with risk of at least one of hemolysis or thrombosis.

### BRIEF DESCRIPTION OF THE DRAWINGS

FIG. 1 illustrates a block diagram of a system for integrating external diagnostics with remote monitoring provided by implantable medical devices in accordance with embodiments herein.

FIG. 2 illustrates a block diagram of a data path that may be implemented in accordance with embodiments herein.

FIG. 3 illustrates examples of various types of diagnostic information that may be collected as Body Generated Analyte data in accordance with embodiments herein.

FIG. 4 illustrates a high-level flowchart of a method, implemented by the medical network of FIG. 1, for processing current or real time BGA data and/or IMD data in accordance with embodiments herein.

FIG. 5A illustrates a healthcare system formed in accordance with embodiments herein.

FIG. 5B illustrates a distributed healthcare system that collects and analyzes patient data in accordance with embodiments herein.

FIG. 6A illustrates a method for collecting new BGA data and based thereon, calculating an BGA Index in accordance with embodiments herein.

FIG. 6B illustrates a method for collecting new IMD data and based thereon, calculating an IMD Index in accordance with embodiments herein.

FIG. 6C illustrates a method for collecting new BRM data and based thereon, calculating a COI in accordance with embodiments herein.

FIG. 6D illustrates a method for generating a treatment diagnosis and treatment recommendation based on real time BGA data and IMD data in accordance with embodiments herein.

FIG. 6E illustrates a process for automatically identifying high risk behavior in HF patients in accordance with embodiments herein.

FIG. 6F illustrates a chart plotting an example of a collection of PAP measurements and PAP trend that may be obtained over time.

FIG. 6G illustrates examples of weekly weight gain/loss trends for a patient population.

FIG. 6H illustrates examples of transmission compliance trend information that may be displayed and/or recorded.

FIG. 6I illustrates examples of PAP trend information.

FIG. 7A illustrates example relationships of increased mortality and worsening nutritional status in accordance with embodiments herein.

FIG. 7B illustrates a graphical representation of a portion of the information that may be stored in a patient medical network in accordance with embodiments herein.

FIG. 7C illustrates examples of pop-up notification windows that may be presented in connection with a patient in accordance with embodiments herein.

FIG. 8A illustrates a comparison of HF prescription changes for a population of patients during a six-month “follow-up” period in accordance with embodiments herein.

FIG. 8B illustrates a timeline in connection with an example medical application that utilizes a PAP sensor in accordance with embodiments herein.

FIG. 8C illustrates a method for implementing an application specific model for diagnosis and treatment recommendations for treating HF patients, who exhibit medication resistance (e.g., diuretic resistance), in accordance with embodiments herein.

FIG. 8D illustrates an operation that may be executed in various temporal relations to one another and in various temporal relations to changes in medication in accordance with embodiments herein.

FIG. 9A illustrates a method for determining a risk score for a patient and predicting a risk of an impending hospitalization for the patient in accordance with embodiments herein.

FIG. 9B illustrates a process for applying a risk score in connection with implementing a treatment diagnoses and recommendations in accordance with embodiments herein.

FIG. 9C illustrates a block diagram of an example method utilized by embodiments herein to determine patient-specific priority rank in accordance with embodiments herein.

FIG. 10A illustrates a method for tracking certain types of IMD data and BGA data indicative of myocardial ischemia and/or myocardial infarctions in accordance with embodiments herein.

FIG. 10B illustrates a method for utilizing the information collected by the operations of FIG. 10A during or in connection with a patient visit to an emergency room or other medical facility in accordance with embodiments herein.

FIG. 11 illustrates a method for managing treatment of patients with a mechanical circulation support device, such as mechanical ventricular assist types of IMDs, in accordance with embodiments herein.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods and systems described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods and systems may be used, in accordance with an embodiment herein. Further, wherein indicated, the methods and systems may be fully or partially implemented by one or more processors of one or more devices or systems. While the operations of some methods and systems may be described as performed by the processor(s) of one device, additionally, some or all of such operations may be performed by the processor(s) of another device described herein.

All references, including publications, patent applications and patents, cited herein are hereby incorporated by reference in their entirety as if each reference were individually and specifically indicated to be incorporated by reference and were set forth in its entirety herein.

Terms

The term “ACEI” shall mean angiotensin-converting enzyme inhibitors.

The term “AF burden” shall mean atrial fibrillation burden. By way of example, the AF burden may be calculated based on the methods and systems described in one or more of the following publications, all of which are expressly incorporated herein by reference in their entireties:
U.S. Patent Publication Number 2014/0275827, entitled “METHOD AND SYSTEM FOR DERIVING EFFECTIVENESS OF MEDICAL TREATMENT OF A PATIENT” published Sep. 18, 2014;
U.S. Patent Publication Number 2014/0039238, entitled “SYSTEMS AND METHODS FOR CONTROLLING NEUROSTIMULATION OF ACUPUNCTURE SITES USING AN IMPLANTABLE CARDIAC RHYTHM MANAGEMENT DEVICE” published Feb. 6, 2014;
U.S. Patent Publication Number 2013/0204147, entitled “ATRIAL FIBRILLATION DETECTION BASED ON PULMONARY ARTERY PRESSURE DATA” published Aug. 8, 2013;
U.S. Patent Publication Number 2013/0116583, entitled “SYSTEMS AND METHODS FOR PREDICTING AND CORROBORATING PULMONARY FLUID OVERLOADS USING AN IMPLANTABLE MEDICAL DEVICE” published May 9, 2013;
U.S. Patent Publication Number 2012/0089032, entitled “METHOD AND SYSTEM FOR DISCRIMINATING AND MONITORING ATRIAL ARRHYTHMIA BASED ON CARDIOGENIC IMPEDANCE” published Apr. 12, 2012;
U.S. Patent Publication Number 2011/0125206, entitled “SINGLE CHAMBER IMPLANTABLE MEDICAL DEVICE FOR CONFIRMING ARRHYTHMIA THROUGH RETROSPECTIVE CARDIAC SIGNALS” published May 26, 2011;
U.S. Patent Publication Number 2014/0221771, entitled “METHOD AND IMPLANTABLE SYSTEM FOR BLOOD-GLUCOSE CONCENTRATION MONITORING USING PARALLEL METHODOLOGIES” published Aug. 7, 2014;
U.S. Patent Publication Number 2014/0058278, entitled “SYSTEMS AND METHODS FOR DETECTING ISCHEMIC EVENTS” published Feb. 27, 2014;
U.S. Patent Publication Number 2013/0218036, entitled “METHODS AND SYSTEMS TO CORRELATE ARRHYTHMIC AND ISCHEMIC EVENTS” published Aug. 22, 2013;
U.S. Patent Publication Number 2013/0184777, entitled “SYSTEMS AND METHODS FOR ASSESSING AND EXPLOITING CONCURRENT CATHODAL AND ANODAL CAPTURE USING AN IMPLANTABLE MEDICAL DEVICE” published Jul. 18, 2013;
U.S. Patent Publication Number 2012/0197149, entitled “SYSTEM AND METHOD FOR DISTINGUISHING AMONG CARDIAC ISCHEMIA, HYPOGLYCEMIA AND HYPERGLYCEMIA USING AN IMPLANTABLE MEDICAL DEVICE” published Aug. 2, 2012;
U.S. Patent Publication Number 2012/0158079, entitled “SYSTEMS AND METHODS FOR ASSESSING THE SPHERICITY AND DIMENSIONAL EXTENT OF HEART CHAMBERS FOR USE WITH AN IMPLANTABLE MEDICAL DEVICE” published Jun. 21, 2012;
U.S. Patent Publication Number 2012/0065527, entitled “METHODS AND SYSTEMS FOR MONITORING ATRIAL STIFFNESS” published Mar. 15, 2012;
U.S. Patent Publication Number 2012/0046528, entitled “SYSTEM AND METHOD FOR DETECTING AND TREATING CARDIOVASCULAR DISEASE” published Feb. 23, 2012; and
U.S. Patent Publication Number 2011/0004111, entitled “ISCHEMIA DETECTION USING INTRA-CARDIAC SIGNALS” published Jan. 6, 2011.

The term “ARB” shall mean angiotensin receptor blockers.

The term “ASM” shall mean application-specific model.

The terms “behavior related medical data” and “BRM data” shall mean information indicative of an action or conduct by a patient that will affect one or more physiologic characteristics of interest and/or information indicative of a present state experienced by a patient in connection with a physiologic characteristic of interest. As nonlimiting examples of information indicative of an act or conduct, BRM data may represent information related to a patient's diet (e.g., what, when and how much a patient ate or drank), information related to whether a patient is following a physician's instructions (e.g., exercising, walking, following a fluid regiment, taking medication at prescribed times), information related to nutritional supplements (e.g., what, when and how much a patient is taking as nutritional supplements), self-reported quality of life information from the patient, signs and symptoms indicating fatigue, lack of mobility/exercise and the like. The foregoing examples concern BRM data that is directly relate to actions and/or conduct by the patient. Optionally, the BRM data may indirectly relate to actions and/or conduct by the patient. For example, the BRM data may indicate how often and/or volumes of certain food products and liquids ordered by the patient through a home delivery service (e.g., how often and in what volume the patient orders certain groceries and other food products that may be delivered to a patient's home).

Further, as nonlimiting examples of information indicative of a present state, the BRM data may represent information indicating how a patient feels (e.g., headaches, shortness of breath, tired, chest pains). The BRM data may be manually entered by the patient or a third-party through various types of PDE devices. Optionally, the BRM data may be automatically entered by a PDE device based on electronic monitoring of actions and conduct by the patient, as well as other types of sensors.

The term “BGA test device” shall mean any and all equipment, devices, disposable products utilized to collect and analyze a BGA. The BGA test device may implement one or more of the methods, devices and systems described in the following publications, all of which are incorporated herein by reference in their entireties:
U.S. Pat. No. 8,514,086, entitled “DISPLAYS FOR A MEDICAL DEVICE”, issued Aug. 20, 2013;
U.S. Patent Publication Number 2011/0256024, entitled “MODULAR ANALYTE MONITORING DEVICE”, published Oct. 20, 2011;
U.S. Patent Publication Number 2010/0198142, entitled “MULTIFUNCTION ANALYTE TEST DEVICE AND METHODS THEREFORE”, published Aug. 5, 2010;
U.S. Patent Publication Number 2011/0160544, entitled “SYSTEM AND METHOD FOR ANALYSIS OF MEDICAL DATA TO ENCOURAGE HEALTHCARE MANAGEMENT”, published Jun. 30, 2011;
U.S. Pat. No. 5,294,404, entitled “REAGENT PACK FOR IMMUNOASSAYS” issued Mar. 15, 1994;
U.S. Pat. No. 5,063,081, entitled “METHOD OF MANUFACTURING A PLURALITY OF UNIFORM MICROFABRICATED SENSING DEVICES HAVING AN IMMOBILIZED LIGAND RECEPTOR” issued Nov. 5, 1991;
U.S. Pat. No. 7,419,821, entitled “APPARATUS AND METHODS FOR ANALYTE MEASUREMENT AND IMMUNOASSAY” issued Sep. 2, 2008;
U.S. Patent Publication Number 2004/0018577, entitled “MULTIPLE HYBRID IMMUNOASSAYS” published Jan. 29, 2004;
U.S. Pat. No. 7,682,833, entitled “IMMUNOASSAY DEVICE WITH IMPROVED SAMPLE CLOSURE” issued Mar. 23. 2010;
U.S. Pat. No. 7,723,099, entitled “IMMUNOASSAY DEVICE WITH IMMUNO-REFERENCE ELECTRODE” issued May 25, 2010; and
Baj-Rossi et al. “FABRICATION AND PACKAGING OF A FULLY IMPLANTABLE BIOSENSOR ARRAY”, (2013) IEEE, pages 166-169.

The term “BNP” shall mean a brain natriuretic peptide. The BNP is determined from a blood test that measures levels of a protein called BPN that is made by the heart and blood vessels. A level for the BNP increases above normal when an individual experiences heart failure.

The term “body generated analyte” shall mean a test substance or specimen that is naturally generated by or naturally present in a human body. The test substance or specimen may be in liquid form (e.g., blood or other bodily fluid), solid form (e.g., tissue, fat, muscle, bone, or other organ-based material), gas form, cellular form or otherwise. Non-limiting examples of body generated analytes include hematocrit, troponin, CKMB, BNP, beta human chorionic gonadotropin (bHCG), carbon dioxide partial pressure (pCO.sub.2), partial pressure oxygen (pO.sub.2), pH, PT, ACT, activated partial thromboplastin time (APTT), sodium, potassium, chloride, calcium, urea, glucose, creatinine, lactate, oxygen, and carbon dioxide, thyroid stimulating hormone, parathyroid hormone, D-dimer, prostate specific antibody, TCO₂, Anion Gap, ionized calcium, urea nitrogen, lactose, hemoglobin, pH, PCO₂, PO₂, HCO₃, Base Excess, O₂, ACT Kaolin, ACT Celite, PT/INR, β-hCG, cTnl, CK-MB, BNP and the like, and combinations thereof. The analyte may be tested in a liquid sample that is whole blood, however other samples can be used including blood, serum, plasma, urine, cerebrospinal fluid, saliva and amended forms thereof. Amendments can include diluents and reagents such as anticoagulants and the like.

The terms “body generated analyte-based index” and “BGA index” shall mean an index that is calculated based on one or more body generated analytes, where the index is indicative of a state of one or more physiologic characteristics of interest (COI) of the patient. As non-limiting examples, the physiologic COI may relate to diuretic response, CRS, intravascular volume depletion/overload, total body overload, malnutrition, peripheral edema, adenomatous GI tract absorption, liver congestion, liver protein generation state, hypoglycemic, hyperglycemic and the like.

The term “BUN” shall mean blood urea nitrogen.

The term “CDS” shall mean chronic disease state.

The term “CDSR” shall mean chronic disease state related.

The term “CONUT” shall mean controlling nutritional status index.

The term “CRI” shall mean congestion related index.

The term “CSI” shall mean chronic disease state index.

The terms “diagnosis” and “diagnosis recommendation” shall mean the identification of the nature of an illness or other problem by examination of the symptoms.

The term “EDT” shall mean an external diagnostic test.

The term “HF” shall mean heart failure.

The term “GFR” shall mean glomerular filtration rate. The Glomerular filtration rate is determined from a test used to check how well the kidneys are working. Specifically, the GFR estimates how much blood passes through the glomeruli per unit time.

The term “GNRI” shall mean geriatric nutrition risk index.

The term “IMD” shall mean an implantable medical device. Embodiments may be implemented in connection with one or more implantable medical devices (IMDs). Non-limiting examples of IMDs include one or more of neurostimulator devices, implantable leadless monitoring and/or therapy devices, and/or alternative implantable medical devices. For example, the IMD may represent a cardiac monitoring device, pacemaker, cardioverter, cardiac rhythm management device, defibrillator, neurostimulator, leadless monitoring device, leadless pacemaker and the like. The IMD may measure electrical and/or mechanical information. For example, the IMD may include one or more structural and/or functional aspects of the device(s) described in U.S. Pat. No. 9,333,351, entitled “NEUROSTIMULATION METHOD AND SYSTEM TO TREAT APNEA” issued May 10, 2016 and U.S. Pat. No. 9,044,610, entitled “SYSTEM AND METHODS FOR PROVIDING A DISTRIBUTED VIRTUAL STIMULATION CATHODE FOR USE WITH AN IMPLANTABLE NEUROSTIMULATION SYSTEM” issued Jun. 2, 2015, which are hereby incorporated by reference. The IMD may monitor transthoracic impedance, such as implemented by the CorVue algorithm offered by St. Jude Medical. Additionally or alternatively, the IMD may include one or more structural and/or functional aspects of the device(s) described in U.S. Pat. No. 9,216,285, entitled “LEADLESS IMPLANTABLE MEDICAL DEVICE HAVING REMOVABLE AND FIXED COMPONENTS” issued Dec. 22, 2015 and U.S. Pat. No. 8,831,747, entitled “LEADLESS NEUROSTIMULATION DEVICE AND METHOD INCLUDING THE SAME” issued Sep. 9, 2014, which are hereby incorporated by reference. Additionally or alternatively, the IMD may include one or more structural and/or functional aspects of the device(s) described in U.S. Pat. No. 8,391,980, entitled “METHOD AND SYSTEM FOR IDENTIFYING A POTENTIAL LEAD FAILURE IN AN IMPLANTABLE MEDICAL DEVICE” issued Mar. 5, 2013 and U.S. Pat. No. 9,232,485, entitled “SYSTEM AND METHOD FOR SELECTIVELY COMMUNICATING WITH AN IMPLANTABLE MEDICAL DEVICE” issued Jan. 5, 2016, which are hereby incorporated by reference. Additionally or alternatively, the IMD may be a subcutaneous IMD that includes one or more structural and/or functional aspects of the device(s) described in U.S. application Ser. No. 15/973,195, entitled “SUBCUTANEOUS IMPLANTATION MEDICAL DEVICE WITH MULTIPLE PARASTERNAL-ANTERIOR ELECTRODES” filed May 7, 2018; U.S. application Ser. No. 15/973,219, entitled “IMPLANTABLE MEDICAL SYSTEMS AND METHODS INCLUDING PULSE GENERATORS AND LEADS” filed May 7, 2018; U.S. application Ser. No. 15/973,249, entitled “SINGLE SITE IMPLANTATION METHODS FOR MEDICAL DEVICES HAVING MULTIPLE LEADS”, filed May 7, 2018, which are hereby incorporated by reference in their entireties. Further, one or more combinations of IMDs may be utilized from the above incorporated patents and applications in accordance with embodiments herein. Embodiments may be implemented in connection with one or more subcutaneous implantable medical devices (S-IMDs). For example, the S-IMD may include one or more structural and/or functional aspects of the device(s) described in U.S. application Ser. No. 15/973,219, entitled “IMPLANTABLE MEDICAL SYSTEMS AND METHODS INCLUDING PULSE GENERATORS AND LEADS”, filed May 7, 2018; U.S. application Ser. No. 15/973,195, entitled “SUBCUTANEOUS IMPLANTATION MEDICAL DEVICE WITH MULTIPLE PARASTERNAL-ANTERIOR ELECTRODES”, filed May 7, 2018; which are hereby incorporated by reference in their entireties. The IMD may represent a passive device that utilizes an external power source, and entirely mechanical plan will device, and/or an active device that includes an internal power source. The IMD may deliver some type of therapy/treatment, provide mechanical circulatory support and/or merely monitor one or more physiologic characteristics of interest (e.g., PAP, CA signals, impedance, heart sounds).

The terms “IMD-based index” and “IMD index” shall mean an index that is calculated based on data collected by an IMD, where the index is indicative of a state of one or more physiologic characteristics of interest of the patient. Nonlimiting examples of signals that may be collected by an IMD and utilized to calculate an IMD Index includes electrocardiogram signals (ECG), pulmonary arterial pressure signals, impedance measurements, right side filling pressure, trans-renal pressure gradient, cardiac output, neurological signals, and hemodynamic signals.

The term “IMD data” shall refer to any and all types of information and signals conveyed from an implantable medical device to a local or remote external device. Nonlimiting examples of IMD data include cardiac activity signals (e.g., intracardiac electrogram or IEGM signals), impedance signals (e.g., cardiac, pulmonary or transthoracic impedances), accelerometer signatures (e.g., activity signals, posture/orientation signals, heart sounds), pulmonary arterial pressure signals, MCS rpm levels, MCS flow rates, device alerts and the like.

The term “INR” shall mean the international normalized ratio which represents a laboratory measurement of how long it takes blood to form a clot.

The term “MCS” shall mean mechanical circulatory support. For example, nonlimiting examples of MCS devices include ventricular assist devices. Nonlimiting examples of MCS data include RPM level, flow level, flow rate, device alerts, changes in RPM and/or flow levels, and the like.

The term “MP” shall mean medical personnel, nonlimiting examples of which include doctors, nurses, hospital or clinical staff, pharmacist, physical therapist, and any other person trained or licensed to provide medical assistance to a patient.

The term “MSI” shall mean malnutrition state index.

The terms “MS body generated analyte” and “MS BGA” shall mean a body generated analyte that exhibits one or more characteristics of interest related to a patient's malnutrition state.

The term “NT-proBNP” shall mean an N-terminal pro b-type natriuretic peptide. The NT-proBNP is determined from a blood test that measures levels of an N-terminal pro b-type natriuretic peptide that is made by the heart and blood vessels. A level for the NT-proBNP increases above normal when an individual experiences heart failure.

The term “obtain” or “obtaining”, as used in connection with data, signals, information and the like, includes at least one of i) accessing memory of an external device or remote server where the data, signals, information, etc. are stored, ii) receiving the data, signals, information, etc. over a wireless communications link between the IMD and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

The term “ONS” shall mean oral nutritional supplement.

The term “PA” shall mean pulmonary artery.

The term “PAP” shall mean pulmonary arterial pressure.

The terms “patient data entry device” and “PDE device” shall mean an electronic device that includes a user interface that is configured 1) to receive patient data that is entered by the patient and/or 2) to receive patient data in connection with actions/decisions by the patient. A PDE device is different from an IMD and a BGA test device. The PDE device is configured to receive behavior related medical data that differs from IMD data and that differs from BGA data. The PDE devices may include, but are not limited to, smart phones, desktop or laptop computers, tablet devices, smart TVs, fixed cameras, smart watch, wearable heart rate monitor, portable or handheld cameras, recording devices, digital personal assistant (DPA) devices and the like. One nonlimiting example of a PDE device is a smart phone implementing the “HEMAAPP” application, developed at the University of Washington, where the application is configured to utilize a camera within a smart phone to monitor a patient's hemoglobin level (while holding a finger over the camera) and to detect when the patient is in an anemic or other undesirable state related to hemoglobin levels. Another example is a smart phone application developed by Wilbur Lam at the Aflac Cancer and Blood Disorders Center of Children's Healthcare of Atlanta, and Wallace Coulter, a faculty member in the Department of biomedical engineering at Georgia Tech. The PDE device may include an electronic device sold under the trademark ALEXA® by Amazon.com Inc., and/or an electronic device sold under the trademark NOW® by Google LLC., and the like. In addition, the PDE devices may represent various types of devices configured to record audio and/or voice signatures, detect gestures and movements and the like. The PDE device may include a graphical user interface, through which the patient or another user enters the patient data. Optionally, the PDE device may include audio and/or video sensors/cameras that may receive patient data. For example, a user may use a keyboard, touch screen and/or mouse to enter patient data. Optionally, the user may enter the patient data through spoken words (e.g., “Alexa I just took my medication”, “Alexa I am eating 3 slices of peperoni pizza”, “Alexa I am eating an apple”, “Alexa I am drinking a 12 oz. soda and eating a candy bar). Optionally, the PDE device may automatically track actions by a patient, such as through the use of cameras to visually watch a patients actions, through the use of microphones to “listen” to a patient's actions, and/or through the use of other types of sensors (e.g., refrigerator or kitchen cabinet door sensor, sensor on a treadmill). For example, a camera may capture video that is processed by a processor utilizing image recognition to identify what a patient is eating/drinking, when the patient eats/drinks, and how much the patient consumed. Optionally, the BRM device may include a position tracking device sold under the trademark FITBIT® by Fitbit Inc. or other types of position tracking devices. The position tracking device may monitor and collect, as BRM data, movement information, such as a number of steps or distance traveled in a select period of time, a rate of speed, a level of exercise and the like. Optionally, the BRM device may monitor and collect, as BRM data, heart rate.

The term “PNI” shall mean prognostic nutritional index.

The term “POC” shall mean point-of-care. The term “point-of-care” and “POC”, when used in connection with medical diagnostic testing, shall mean methods and devices configured to provide medical diagnostic testing at or near a time and place of patient care. The time and place of patient care may be at an individual's home, such as when providing “at home” point of care solutions. The time and place of patient care may be at a physician's office or other medical facility, wherein one or more medical diagnostic tests may be performed on-site at a time of or shortly after a patient visit and collection of a patient sample. The POC may implement the methods, devices and systems described in one or more of the following publications, all of which are expressly incorporated herein by reference in their entireties:
U.S. Pat. No. 6,786,874, entitled “APPARATUS AND METHOD FOR THE COLLECTION OF INTERSTITIAL FLUIDS” issued Sep. 7, 2004;
U.S. Pat. No. 9,494,578, entitled “SPATIAL ORIENTATION DETERMINATION IN PORTABLE CLINICAL ANALYSIS SYSTEMS” issued Nov. 15, 2016; and
U.S. Pat. No. 9,872,641, entitled “METHODS, DEVICES AND SYSTEMS RELATED TO ANALYTE MONITORING” issued Jan. 23, 2018.

The term “primary”, when used in connection with a relation between a treatment and a physiologic COI, shall mean a physiologic COI that the treatment is intended to affect by changing the physiologic COI from an undesired level to a desired level. For example, a treatment may adjust a diuretic prescription to increase/decrease the primary physiologic COI of PAP from an unduly high or low PAP to a normal PAP.

The term “probability” shall mean, not only a determined percentage or numerical value, but also non-numerical values and corresponding indicators. For example, a risk score algorithm may be used to determine a risk range or risk category a patient falls into with the range or risk category illustrated by color, bars, and the like. Such probability may also include morphological comparisons such as comparisons of waveforms in graphs associated with heart related data. In this manner, probability is simply the factoring or use of event related variable(s) to determine the likelihood an event will or won't occur. The probability thus may be represented in numerous manners, including a risk score, a subsequent risk score, a percentage, a bar graph, a range, a color-coded indicator, text indicia providing an indicator text such as “low”, “medium”, and “high”, pictorially, and the like.

The terms “processor,” “a processor”, “one or more processors” and “the processor” shall mean one or more processors. The one or more processors may be implemented by one, or by a combination of more than one implantable medical device, a wearable device, a local device, a remote device, a server computing device, a network of server computing devices and the like. The one or more processors may be implemented at a common location or at distributed locations. The one or more processors may implement the various operations described herein in a serial or parallel manner, in a shared-resource configuration and the like.

The term “real-time” shall refer to a time period substantially contemporaneous with an event of interest. The term “real-time,” when used in connection with collecting and/or processing data utilizing an IMD, shall refer to processing operations performed substantially contemporaneous with a physiologic event of interest experienced by a patient. By way of example, in accordance with embodiments herein, cardiac activity signals are analyzed in real time (e.g., during a cardiac event or within a few minutes after the cardiac event). The term “real-time,” when used in connection with a body generated analyte, shall refer to operations performed substantially contemporaneous with an occurrence of a characteristic of interest in a malnutrition state experienced by the patient. By way of example, in accordance with embodiments herein, the body generated analyte may correspond to serum albumin that is analyzed and utilized in a diagnosis and treatment recommendation. The analysis of the serum albumin and generation of the diagnosis and treatment recommendation are performed in real-time, namely while the patient is experiencing a certain malnutrition state, not to exceed 24 hours from the time the BGA was collected.

The term “secondary”, when used in connection with a relation between a treatment and a physiologic COI, shall mean a physiologic COI that the treatment is not intended to affect. For example, when the treatment adjusts the diuretic prescription, a secondary physiologic COI may be renal function that is already operating in a normal or acceptable manner, and should not be affected by the diuretic change, but nonetheless may be adversely affected by the diuretic change.

The term “treatment notification” shall mean a communication and/or device command to be conveyed to one or more individuals and/or one or more other electronic devices, including but not limited to, network servers, workstations, laptop computers, tablet devices, smart phones, IMDs, EDT equipment and the like. When a treatment notification is provided as a communication, the treatment notification may represent in an audio, video, vibratory or other user perceivable medium. The communication may be presented in various formats, such as to display patient information, messages, user directions and the like. The communication is presented on one or more of the various types of electronic devices described herein and may be directed to a patient, a physician, various medical personnel, various patient record management personnel and the like. The communication may represent an identification of a patient diagnosis and various treatment recommendations. The diagnosis and treatment recommendation may be provided directly to the patient. For example, in some circumstances, a diagnosis and treatment recommendation may be to modify a dosage level, in which case, the notification may be provided to the physician or medical practitioner. As another example, the diagnosis and treatment recommendation may be to begin, change or end certain physical activities, in which case, the notification may be provided to the patient, in addition to the physician or medical practitioner. As another example, the treatment notification may present an indication that a patient may or may not be a good candidate suited for implant of a ventricular assist device (e.g., LV assist device), a transplant, a valve repair procedure (e.g., a MitraClip™ valve repair to correct mitral regurgitation) and the like. Other nonlimiting examples of a communication type notification include, in part or in whole, a recommendation to schedule an appointment with a physician, schedule an appointment for additional blood work, perform an additional at home POC blood analysis (e.g., utilizing at home EDT equipment), recommend that the patient collect additional EDT and/or IMD data. When a notification includes an action that may be performed by a patient alone, the notification may be communicated directly to the patient. Other nonlimiting examples of a communication type notification include communications sent to a patient (e.g., via a PDE device or other electronic device), where the communication informs the patient of how a patient's lifestyle choices are directly affecting the patient's health. For example, when a patient consumes too much sugar, a notification may be sent to the patient to inform that the excessive sugar has caused a spike in the patient's glucose level. As another example, when a patient avoids exercise for a period of time, the notification may inform a patient that the patient's lack of exercise has raised a PAP trend and/or introduced an undue burden on a patient's kidneys.

When a treatment notification is provided as a device command, the treatment notification may represent an electronic command directing a computing device (e.g., IMD, EDT equipment, local external device, server) to perform an action. For example, the action may include directing the following:
1. IMD or EDT equipment to provide additional IMD data and/or EDT data already available;
2. IMD or EDT equipment to collect additional data and/or another type of data;
3. IMD to deliver a therapy and/or modify a prior therapy (e.g., a pacing therapy, neural stimulation therapy, appetite suppression therapy, drug delivery rate);
4. Local external device to provide additional information regarding past and present behavior of the patient; and
5. Server to analyze further information in the patient medical record and/or from another medical record.

The term “treatment recommendation” shall mean a recommendation for the patient, medical personnel and/or a device (e.g., an IMD, local external device, remote server, or BGA device) to take an action and/or maintain a current course of action. Non-limiting examples of treatment recommendations include dispatching an ambulance to the patient's location, instructing the patient immediately go to a hospital, instructing the patient schedule an appointment, instructing the patient change a prescription, instructing the patient undergo additional examinations (e.g., diagnostic imaging examinations, exploratory surgery and the like), instructing the patient undergo a POC test to collect new BGA data, instructing the patient take a nutritional supplement (e.g., an ONS), instructing the patient start, stop or change a physical activity, or instructing the patient make no changes. The treatment recommendation may include an instruction to change, maintain, add or stop a therapy delivered by an active IMD, such as a pacing therapy, and ATP pacing therapy, a neural stimulation therapy, mechanical circulatory support and the like.

### System Overview

FIG. 1 illustrates a block diagram of a system 100 for integrating external diagnostics with remote monitoring of data provided by implantable medical devices in accordance with embodiments herein. The system may be implemented with various architectures, that are collectively referred to as a healthcare system 120. By way of example, the healthcare system 120 may be implemented as described herein. The healthcare system 120 is configured to receive data from a variety of external and implantable sources including, but not limited to, active IMDs 102 capable of delivering therapy to a patient, passive IMDs or sensors 104, BGA test devices 106, wearable sensors 108, and point-of-care (POC) devices 110 (e.g., at home or at a medical facility). A POC device 110 may represent one type of BGA test device 106. The data from one or more of the external and/or implantable sources is collected and transmitted to one or more secure databases within the healthcare system 120. Optionally, the patient and/or other users may utilize a PDE device, such as a smart phone, tablet device, etc., to enter BRM data. For example, a patient may use a smart phone to provide feedback concerning activities performed by the patient, a patient diet, nutritional supplements and/or medications taken by the patient, how a patient is feeling (e.g., tired, dizzy, weak, good), etc.

For example, the external BGA test device 106 may collect lab test results for specific tests and then transmit the lab test results to the healthcare system 120. The BGA test device 106 may be implemented at a variety of physical locations, such as one or more “core” laboratories, a physician's office, ER (emergency room), OR (operating room) and/or a medical facility POC (e.g., during hospitalizations or routine healthcare visits). The BGA test device 106 may be implemented as an at-home POC device 110 that collects test results periodically or continuously monitor one or more body generated analytes (e.g., blood glucose). The at home POC device 110 may transmit the raw BGA data to the medical network (e.g., a local external device and/or remote server). Additionally or alternatively, the at-home POC device 110 may implement a corresponding test of the BGA data for a characteristic of interest (COI) such as a malnutrition state COI, an electrolyte COI, a cardiac marker COI, a hematology COI, a blood gas COI, a coagulation COI, an endocrinology COI. The POC device 110 transmits the COI (and optionally the BGA data) to the healthcare system 120 as the tests are performed at home or elsewhere. The POC device 110 may implement periodic or continuous tests for glucose levels, such as through sensors and handheld devices offered under the trademark FREESTYLE LIBRE® by Abbott Laboratories. Optionally, the BGA test device 106 may be implemented as a fully implantable “lab on a chip”, such as an implantable biosensor array, that is configured to collect lab test results.

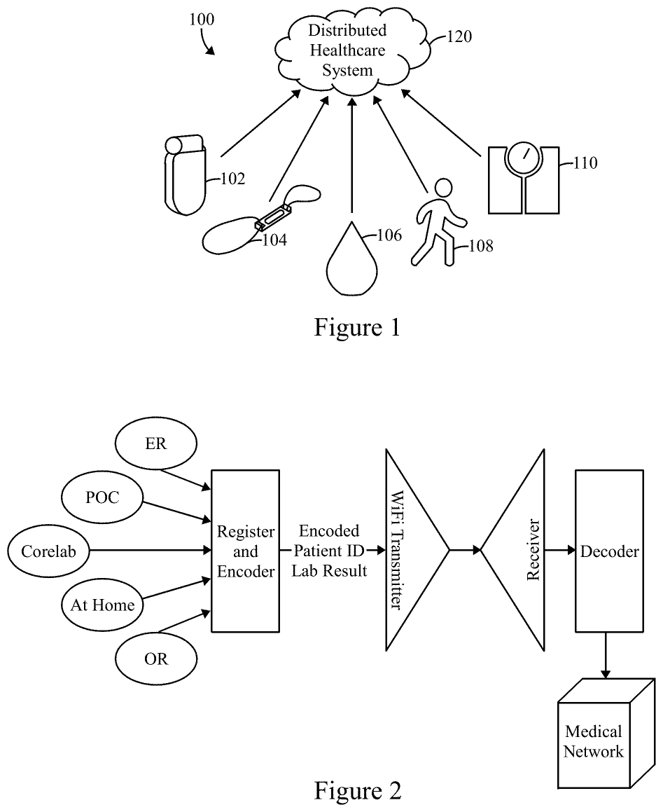

FIG. 2 illustrates a block diagram of a data path that may be implemented in accordance with embodiments herein to encode/decode IMD and/or BGA data during transfer to and within the healthcare system 120. FIG. 2 illustrates various physical locations, at which data may originate, such as one or more “core” laboratories, a physician's office, ER (emergency room), OR (operating room) and/or a medical facility POC location (e.g., during hospitalizations or routine healthcare visits), at-home POC location and the like. The devices (BGA test device, IMD, local external device, MP device) include an encoder and register to store encoded patient IDs, IMD data and BGA data. The encoded IMD and BGA data is stored in one or more data storage devices. The IMD and BGA data are transmitted over various types of networks to a data storage within the healthcare system 120. The data storage device within the healthcare system 120 may represent one or more databases and/or servers located at a single central location. Optionally, the data storage device may represent one or more databases and/or servers distributed across multiple locations throughout a city, throughout a state, throughout a country and/or worldwide. The patient data (e.g., IMD data, BGA data, diagnostic imaging data, and other medical data) for an individual patient may be centrally stored at one location and/or distributed across multiple data storage devices throughout the system. For example, IMD data for a patient may be maintained at one or more locations, such as a central network server configured to communicate and coordinate with the corresponding type of IMD. The BGA data for the same patient may be maintained at one or more other locations, such as at a different central network server configured to communicate and coordinate with the corresponding type of BGA test device. The IMDs, BGA test devices, local external devices, servers and MP devices are provided with one or more decoders that are configured to or decode patient IDs, IMD data and BGA data.

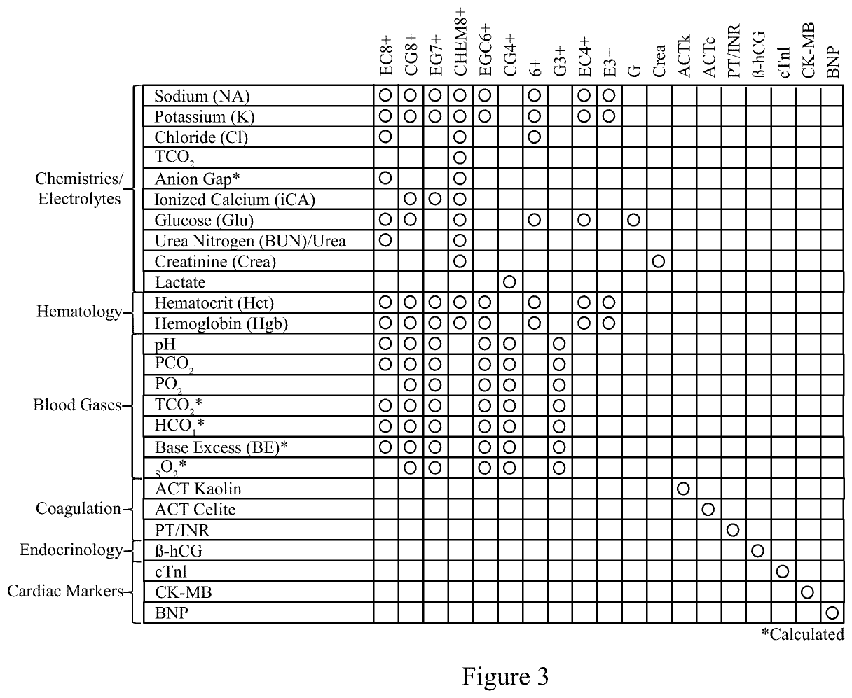

FIG. 3 illustrates examples of various types of diagnostic information that may be collected as BGA data. For example, a single-use cartridge may be utilized for a range of clinical tests, including cardiac markers, cardiac enzymes, lactate, coagulation, blood gases, chemistries and electrolytes, and hematology. As nonlimiting examples, a cartridge based BGA test device may be configured to test patient levels for one or more body generated analytes. The system may offer a comprehensive range of clinical tests in a single point-of-care device and provide medical diagnostic test results to the methods and systems herein in a time-sensitive manner.

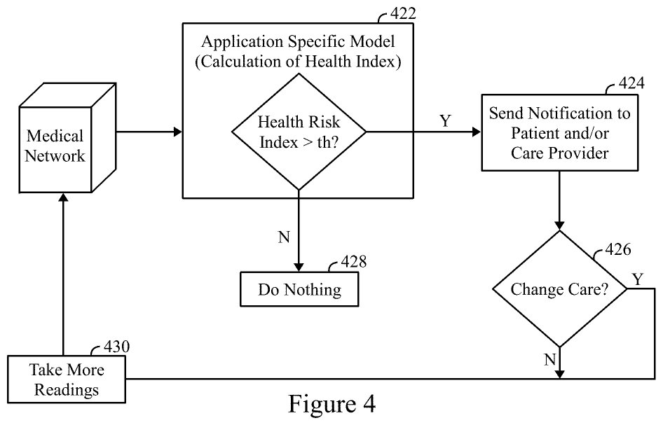

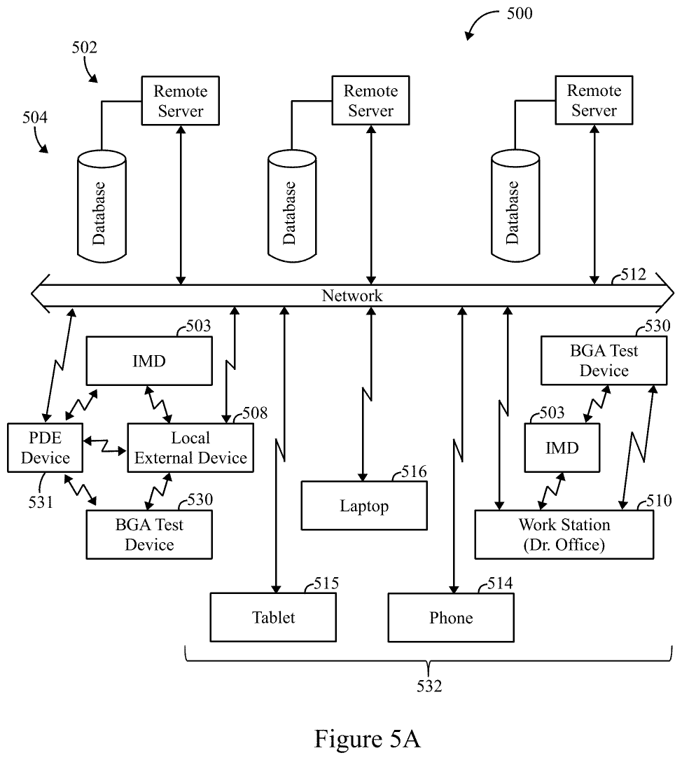

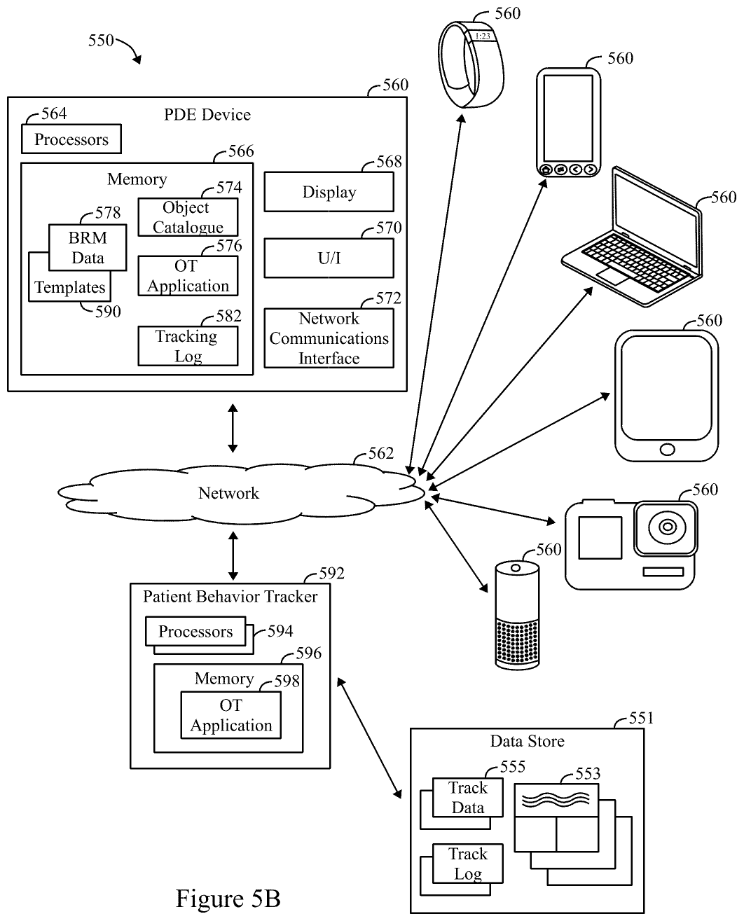

FIG. 4 illustrates a high-level flowchart of a method, implemented by the medical network of FIG. 1, for processing current or real time BGA data and/or IMD data in accordance with embodiments herein. The healthcare system 120 includes one or more computing devices (e.g., servers, local external devices, MP devices as shown in FIG. 5A) that are configured to collect and process BGA data and/or IMD data. The example of FIG. 4 represents an example of a high-level analysis that may be implemented, with more detailed examples provided herein. Upon receipt of new (or changes in) BGA data and/or IMD data, at 422, the processor of the computing device(s) identifies one or more application specific models or ASM to analyze the BGA and/or IMD data. Optionally, the analysis by the ASM may incorporate the additional BGA and/or IMD data into any relevant trend tracked in connection with the present patient. For example, the ASM may track trends in mean PAP, systolic PAP, dicrotic notch in the PAP waveform, change in PAP over time (dPAP/dt), AF burden, arrhythmia burden, etc. The application specific model may be implemented in various manners, as described herein, including but not limited to lookup tables, decision trees, machine learning algorithms and the like. At 422, the processor calculates a health risk index based on the incoming BGA and/or IMD data, alone or in combination with previously stored BGA and IMD data. The health risk index represents a general indicator of a degree to which the patient is experiencing a health state or potential health risk. As a patient's health deteriorates, indicated by one or more characteristics reflected in the BGA and IMD data, the health risk index will similarly elevate. At 422, the processor also generates a treatment diagnosis based on the IMD data and BGA data.

At 422, the processor also determines whether the health risk index exceeds one or more thresholds. When the health risk index does not exceed the threshold(s), the process interprets the condition as an indication that the incoming BGA and/or IMD data indicates that a patient's health condition remains relatively stable. This is assessed as either an instantaneous change relative to the last health risk index, or based on a gradual increase in the health risk index over a pre-defined period of time. Accordingly, flow moves to 428 where the process determines that no other action is necessary. Alternatively, when the health risk index exceeds the threshold, the process interprets the condition as an indication that the incoming BGA and/or IMD data indicates that a patient's health condition is deteriorating and in connection there with flow moves to 424.

At 424, the processor generates a treatment notification based on the treatment diagnosis and directs the treatment notification to be sent to the patient and/or a care provider. At 426, the one or more processors determine whether a change in care has been identified by the treatment diagnosis. Optionally, the operation at 426 may be implemented manually by a clinician or other medical practitioner. As a further option, the operation at 426 may be implemented automatically by one or more processors, as well as manually by a clinician or medical practitioner. The clinician or medical practitioner may then be afforded an option to “override” or modify the automated determination of a change in care. For example, the notification may include a treatment notification such as an indication that a patient may or may not be a good candidate suited for implant of a ventricular assist device (e.g., LV assist device), a transplant, a valve repair procedure (e.g., a MitraClip™ valve repair to correct mitral regurgitation) and the like.

At 430, the processor determines whether to obtain additional BGA and/or IMD data and the process continues by collecting additional data. For example, the operation at 430 may simply represent a continuous loop at which the healthcare system 120 waits to receive new/additional BGA and/or IMD data. Additionally or alternatively, the processor may determine (e.g., as part of the treatment diagnosis) that further data should be obtained before a change in care is decided. Optionally, the operation at 430 may be implemented manually by a clinician or other medical practitioner. As a further option, the operation at 430 may be implemented automatically by one or more processors, as well as manually by a clinician or medical practitioner. The clinician or medical practitioner may then be afforded an option to “override” or modify the automated determination to obtain additional BGA and/or IMD data. The process of FIG. 4 automatically develops the treatment diagnosis (e.g., clinical insights) based on all available data. The clinical insights may result in a determination to i) collect more data, ii) recommend a change in clinical care or otherwise. Optionally, a clinician may be afforded the option to “Opt-In” or “Opt-out” of one or more different features and applications, thereby allowing the clinicians to choose which clinical insights they receive in connection with managing patients. Optionally, a clinician may be afforded the option to make decisions (e.g., render a diagnosis, change treatment, collect more data) and/or validate/reject decisions rendered automatically by the one or more processors.

Optionally, the process of FIG. 4 may be implemented in whole or in part within one or more IMD, a PDE and/or a local external computing device. For example, an IMD may track the IMD data and detect possible deterioration of patient's health. When the IMD detects possible deterioration, the IMD notifies the patient to perform a POC measurement to collect supplemental BGA data. Optionally, when the IMD detects possible deterioration, the IMD may automatically convey a device command (as a treatment notification) to a BGA test device. In response, the BGA test device may automatically collect supplemental BGA data. The combination of the IMD data and the supplemental BGA data is analyzed in accordance with embodiments herein locally or remotely. Additionally or alternatively, the BGA test device may perform continuous BGA monitoring (e.g., instructions patient to take PAP measurement, etc.) and locally analyze the BGA data for indications of possible deterioration in a patient condition. When the BGA test device identifies a possible deterioration in a patient condition, the BGA test device may automatically convey a device command to an IMD to direct the IMD to collect supplemental IMD data. The combination of the BGA data and the supplemental IMD data is analyzed in accordance with embodiments herein locally or remotely.

The system of FIG. 4 may be implemented in whole or in part in connection with tracking patients during a general health risk to the public, such as a pandemic (e.g. the COVID-19 pandemic, another coronavirus pandemic and the like). For example, the ASM may identify patients who have tested positive for an infectious disease, such as for example, a coronavirus, inform various care providers, inform the patient, implement various contact tracing procedures and the like. As one example, the system of FIG. 4 may collect geographic information for a population of patients who have tested positive for the coronavirus and based thereon identify “hotspots” and the like. The geographic information may be collected in various manners, such as just based on the patient's address or other historical medical information, based on GPS coordinates provided from a smart phone or other portable device that supports tracking. As another example, a patient being monitored by the system of FIG. 4 may not test positive for the coronavirus, however, the system may include data indicating that a hotspot has occurred in a geographic area including approximate to a patient. In response thereto, the system may send a notification to the patient informing the patient that the number of positive coronavirus cases has unduly increased in the immediate area or in a surrounding area and provide various directions to the patient to avoid risk of exposure. As a further example, once a patient has been identified as testing positive for an inflammatory disease, such as a coronavirus, the system may subsequently track the condition of the patient and subsequently provide notification to the patient and/or caregivers that it may be desirable to have the patient tested for antibodies or other resistance to the coronavirus.

As another example, the system of FIG. 4 may track a condition of a patient relative to health risk that may be of limited concern to the general public, but of particular concern to the present patient given the present patient's health, such as an immune deficiency, undue susceptibility to inflammatory diseases and the like. For example, the system may identify patients susceptible to a particular inflammatory disease and inform the patient that other individuals in geographic proximity to the patient who have tested positive for the inflammatory disease. As another example, when tracking a geographic location of the patient (e.g. through a smart phone or other portable device), the system may identify that a patient is traveling and is in a geographic area having a high number of positive cases of a coronavirus or other disease to which the patient may be susceptible. The system may push a notification to the patient smart phone including a warning. Various types of alerts may be added and adjusted.

In accordance with new and unique aspects herein, embodiments may direct a patient to collect IMD data, BGA data and/or BRM data more frequently than collected during normal conditions, such as during a public health risk. For example, during normal conditions a patient may obtain a PAP measurement once each day. During a pandemic or other public health risk, the patient may be directed to increase the frequency of PAP measurements (e.g. every four hours, morning, noon and night, etc.).

In accordance with new and unique aspects herein, embodiments may allow updates to prescriptions that add medications not initially indicated for a patient's original condition. For example, a patient may originally be prescribed various diuretics. However, when a patient contracts an inflammatory disease or other temporary disease, the patient may be prescribed or unilaterally choose to take additional medication (e.g. Hydroxychloroquine, Remdesivir, anti-inflammatory steroids or the like). In response thereto, the system of FIG. 4 may update the ASM to analyze characteristics of the IMD data, BGA data and/or BRM data related to shortness of breath, arrhythmias, poor hemodynamics or other health conditions that may result as side effects from the additional medication. The system may provide warnings and other alerts to the patient regarding the additional medications.

### Healthcare System

FIG. 5A illustrates a healthcare system 500 formed in accordance with embodiments herein. The healthcare system 500 includes one or more servers 502, each of which is connected to one or more database 504. The servers 502 and databases 504 may be located at a common physical location and/or distributed between multiple remote locations within a city, state, country or worldwide. The servers 502 communicate with one or more networks 512. The network 512 may be the internet, a voice over IP (VoIP) gateway, a local plain old telephone service (POTS) such as a public switched telephone network (PSTN), a cellular phone-based network, and the like. Alternatively, the network 512 may be a local area network (LAN), a campus area network (CAN), a metropolitan area network (MAN), or a wide area network (WAM). The network 512 serves to provide a network that facilitates the transfer/receipt of information such as IMD data and BGA data.

The system 500 also includes one or more IMDs 503, one or more local external devices 508, one or more BGA test devices 530, one or more PDE devices 531 and one or more medical personnel (MP) devices 532, all of which communicate (directly or indirectly) through the network 512 to the servers 502 and/or one another. The IMD 503 may be passive or active, may collect various types of data, such as cardiac electrical and/or mechanical activity data, PAP or other pressure related data, impedance data, RPM data, flow data, and the like. The BGA test device 530 may analyze various types of body generated analytes to derive the BGA data. The PDE device 531 may communicate with any or all of the IMDs 503, local external devices 508, a BGA test device 530, as well as the network 512. The PDE device 531 collects BRM data, such as based on manual inputs from a patient or other user, and/or based on automatic video and/or audio monitoring.

The local external device 508 may be implemented as a variety of devices including, but not limited to, medical personnel programmer, a local RF transceiver and a user workstation, smart phone, tablet device, laptop computer, desktop computer and the like. The MP devices 532 may also be implemented as a variety of devices including, but not limited to, medical personnel programmer, workstation, smart phone, tablet device, laptop computer, desktop computer and the like. Functionality of the MP device 532 related to embodiments herein may be implemented through dedicated hardware circuits, firmware, software, and/or application operating on one or more computing devices.

The server 502 is a computer system that provides services to other computing systems over a computer network. The servers 502 control the communication of information including IMD data, patient entered data, medical record information and BGA. The servers 502 interface with the network 512 to transfer information between the servers 502, databases 504, local external devices 508, medical personnel devices 532 for storage, retrieval, data collection, data analysis, diagnosis, treatment recommendations and the like.

The databases 504 store all or various portions of the information described herein, including, but not limited to, IMD data, BGA data, BRM data, medical record information, treatment diagnoses and recommendations, and the like. Various portions of the information may be downloaded or uploaded in combination or separately to/from the databases 504, local external devices 508 and MP devices 532. The local external device 508 may reside in a patient's home, a hospital, or a physician's office. The local external device 508 communicates wired or wirelessly with one or more IMD 503 and/or BGA test devices 530. The servers and devices described herein may wirelessly communicate with one another utilizing various protocols, such as Bluetooth, GSM, infrared wireless LANs, HIPERLAN, 3G, satellite, as well as circuit and packet data protocols, and the like. Alternatively, a hard-wired connection may be used to connect the servers and devices. The local external device 508, when implemented as a programmer, may be configured to acquire cardiac signals from the surface of a person (e.g., ECGs), and/or intra-cardiac electrogram (e.g., IEGM) signals from the IMD 503. The local external device 508 interfaces with the network 512 to upload the data and other information to the server 502.

Optionally, the local external device may represent a local RF transceiver that interfaces with the network 512 to upload IMD data and/or BGA data.

The workstation 510 may interface with the network 512 via the internet or POTS to download various data, information, diagnoses and treatment recommendations from the database 504. Alternatively, the workstation 510 may download raw data from the surface ECG units, leads, or monitoring device via either the programmer or the local RF transceiver. Once the user workstation 510 has downloaded the cardiac signal waveforms, ventricular and atrial heart rates, or detection thresholds, the user workstation 510 may process the information in accordance with one or more of the operations described above. The system may download information and notifications to the cell phone 514, the tablet device 515, the laptop 516, or to the server 502 to be stored on the database 504.

Thus, provided is a distributed “digital” healthcare system that collects various types of data, enables the data to be analyzed by various computing devices within the system and determines one or more treatment diagnosis and treatment recommendation substantially in real-time with the collection of new data. In this manner, unneeded and undesired hospitalizations may be avoided through preventative detection, reducing costs associated with emergency medical procedures. Additionally, such a system also assists in prolonging a human's life and increases patient care. Thus, an improved system and methodology are provided.

Optionally, the health risk index may be utilized to rank and schedule patients for future appointments to ensure those patients with the greatest risk of a medical emergency are monitored more closely than those with less of a risk.

FIG. 5B illustrates a distributed healthcare system 550 that collects and analyzes patient data in accordance with embodiments herein. The system 550 includes one or more patient data entry (PDE) devices 560 that communicate over a network 562 with various other devices, such as IMDs, BGA test devices, MP devices, local external devices, servers and the like. Optionally, the PDE devices 560 may communicate through a wholly or partially wired subsystem. The network 562 may represent the World Wide Web, a local area network, a wide area network and the like. When the PDE device 560 includes a GUI, the patient or other user may input patient data in addition to IMD data and BGA data. Optionally, the PDE devices 560 may include one or more microphones that are configured to listen for audible information spoken by a user or patient, such as a verbal statement to enter patient data. Optionally, the PDE devices 560 may include one or more cameras that are configured to capture still images and/or video that is processed utilizing image recognition to identify what action a patient is performing (e.g., what, when and how much a patient is eating and/or drinking). The PDE device 560 includes one or more processors 564, memory 566, a display 568, a user interface 570, a network communications interface 572, and various other mechanical components, electrical circuits, hardware and software to support operation of the PDE device 560. It is recognized that not all PDE devices 560 include a display, user interface, and the like. For example, a fixed or handheld camera may simply include camera related electronics and network circuitry to support communication to and from the camera.

The user interface 570 is configured to receive behavior related medical (BRM) data related to information indicative of an action or conduct by a patient that will affect one or more physiologic characteristics of interest and/or information indicative of a present state experienced by a patient in connection with a physiologic characteristic of interest. The user interface 570 may include a variety of visual, audio, and/or mechanical devices. For example, the user interface 570 can include a visual input device such as an optical sensor or camera, an audio input device such as a microphone, and a mechanical input device such as a keyboard, keypad, selection hard and/or soft buttons, switch, touchpad, touch screen, icons on a touch screen, a touch sensitive areas on a touch sensitive screen and/or any combination thereof. Similarly, the user interface 570 can include a visual output device such as a liquid crystal display screen, one or more light emitting diode indicators, an audible output device such as a speaker, alarm and/or buzzer, and a mechanical output device such as a vibrating mechanism. The display may be touch sensitive to various types of touch and gestures. As further examples, the user interface 570 may include a touch sensitive screen, a non-touch sensitive screen, a text-only display, a smart phone display, an audible output (e.g., a speaker or headphone jack), and/or any combination thereof. The user interface 570 permits the user to select one or more of a switch, button or icon in connection with various operations of the PDE device 560 in connection with entering the BRM data. As nonlimiting examples, the patient or a third-party (e.g., family member, caregiver) may enter, through the PDE device 560, information related to the patient's diet and/or nutritional supplements (e.g., what, when and how much a patient is taking), information concerning whether a patient is following a physician's instructions, information indicative of a present state experienced by the patient and the like. For example, a user may use a keyboard, touch screen and/or mouse to enter BRM data. Optionally, the user may enter the BRM data through spoken words (e.g., “Alexa I just took my medication”, “Alexa I am eating 3 slices of peperoni pizza”, “Alexa I am eating an apple”, “Alexa I am drinking a 12 oz. soda and eating a candy bar).

Optionally, the PDE device may automatically monitor actions or conduct of interest. For example, a camera may be positioned to have a kitchen in a field of view. Still or video images from the camera are analyzed by one or more processors such as through image recognition to identify what, when and how much a patient eats or drinks. Optionally, the PDE device may include a microphone positioned near a kitchen and/or eating area. The audio recording may be analyzed by one or more processors to identify sounds indicative of eating and/or drinking food products of interest. The results from the analysis of the images and/or audio recording are saved as BRM data are utilized as explained herein. Optionally, the PDE device may automatically track actions by a patient, such as through the use of other types of sensors (e.g., refrigerator or kitchen cabinet door sensor, sensor on a treadmill). Optionally, the PDE device may include a position tracking device sold under the trademark FITBIT® by Fitbit Inc. or other types of position tracking devices. The position tracking device may monitor and collect, as BRM data, movement information, such as a number of steps or distance traveled in a select period of time, a rate of speed, a level of exercise and the like. Optionally, the PDE device may monitor and collect, as BRM data, heart rate.

The memory 566 can encompass one or more memory devices of any of a variety of forms (e.g., read only memory, random access memory, static random access memory, dynamic random access memory, etc.) and can be used by the processor 564 to store and retrieve data. The data that is stored by the memory 566 can include, but need not be limited to, operating systems, applications, and other information, in addition to BRM data 578. Each operating system includes executable code that controls basic functions of the communication device, such as interaction among the various components, communication with external devices via a wireless transceivers and/or component interface, and storage and retrieval of applications and data to and from the memory 566. Each application includes executable code that utilizes an operating system to provide more specific functionality for the communication devices, such as file system service and handling of protected and unprotected data stored in the memory 566.

The network communications interface 572 provides a direct connection to other devices, auxiliary components, or accessories for additional or enhanced functionality, and in particular, can include a USB port for linking to a user device with a USB cable. Optionally, the network communications interface 572 may include one or more transceivers that utilize a known wireless technology for communication.

In connection with embodiments that automatically collect BRM data, the memory 566 includes, among other things, an object tracking (OT) application 576, object catalogue 574, BRM data 578, a tracking log 582 and one or more templates 590. The memory 566 may store pick-up zones, drop-off zones, secure zones and access levels as described herein. The functionality of the OT application 576 is described below in more detail. The templates 590 may include one or more types of templates that are descriptive of, and associated with, food objects, nutritional supplement objects, and other objects of interest. More than one type of template (e.g., images, audio signatures, gestures) may be associated with a single type of object (e.g., vegetables, ice cream, meat products, vitamins, alcohol). For example, image-based templates may include still or video images associated with one type of food object, where the images are taken from different angles, with different lighting, and at different distances from the object. As another example, multiple sets of image-based templates may be stored in connection with multiple objects that are of a similar type (e.g., multiple pictures of different types of vegetables).

The BRM data 578 may be collected over the network 562 from numerous types of PDE devices 560 that implement a tracking operation (also referred to as tracking devices). For example, different types of electronic tracking devices 560 may collect image-based tracking data, audio-based tracking data, voice-based tracking data and gesture-based tracking data. The OT application 576 utilizes the templates 590 to analyze incoming data to identify objects of interest. The OT application 576 updates the tracking log 582 based on the analysis.

In the foregoing example, the PDE device 560 implements the OT application 576 locally on a device that may be generally present within the physical area of a user. For example, the PDE device 560 may represent the user's cell phone, laptop computer, tablet device, DPA device and the like.

Additionally or alternatively, all or portions of the OT application may be implemented remotely on a remote resource, referred to as a patient behavior tracker 592. The patient behavior tracker 592 includes one or more processors 594 that may perform limited operations, such as manage storage and creation of templates. The patient behavior tracker 592 may provide access to one or more memory 594, and/or implement the OT application 598. The patient behavior tracker 592 communicates with PDE devices 560 through one or more networks 562 to provide access to object catalogs and to implement processes described herein. The patient behavior tracker 592 may represent a server or other network-based computing environment. The patient behavior tracker may represent a single computing device, or a collection of computer systems located at a common location or geographically distributed.

The patient behavior tracker 592 includes one or more processors 594 and memory 596, among other structures that support operation thereof. In accordance with embodiments herein, the patient behavior tracker 592 receives requests from various PDE devices 560 and returns resources in connection there with. It is recognized that the patient behavior tracker 592 performs other operations, not described herein, such as operations associated with maintaining resources and the like.

The memory 551 may store the object catalogs 553 organized in various manners and related to a wide variety of objects and types of tracking data. The object catalogs 553 may be organized and maintained within any manner of data sources, such as data bases, text files, data structures, libraries, relational files, flat files and the like. The object catalogs 553 include various types of templates corresponding to different types of objects. Optionally, the memory 551 may store BRM data 555, along with timing information such as when the patient behavior tracker 592 receives the BRM data 555 from PDE devices 560 that are performing tracking operations.

### Process to Collect and Analyze BGA and IMD Data

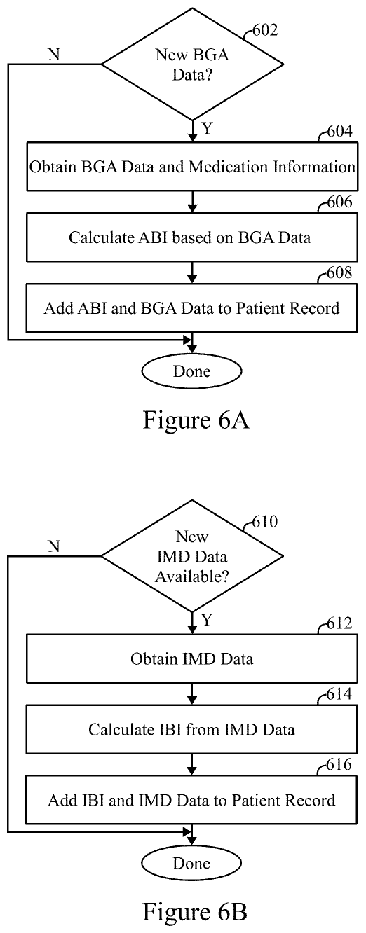

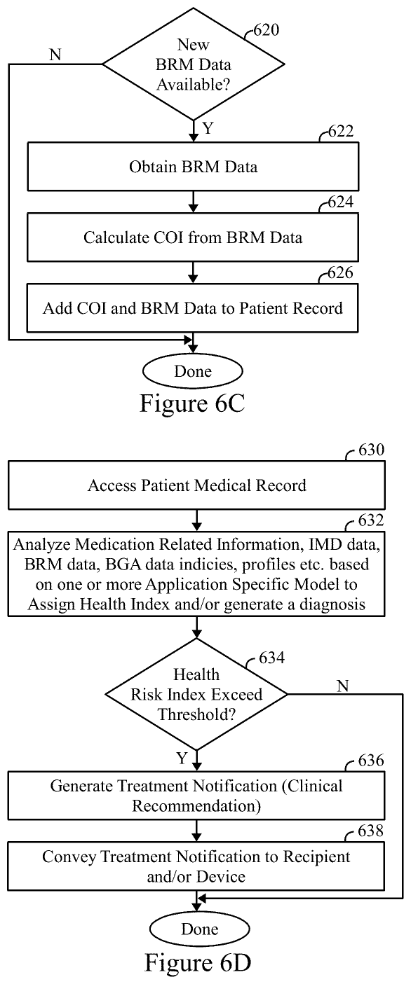

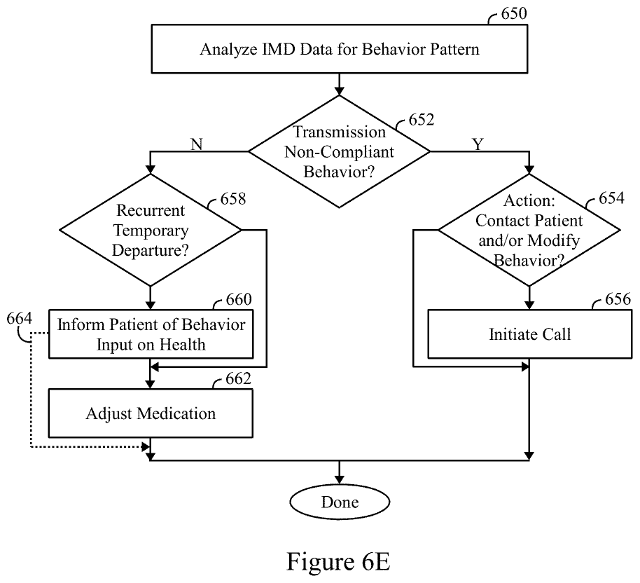

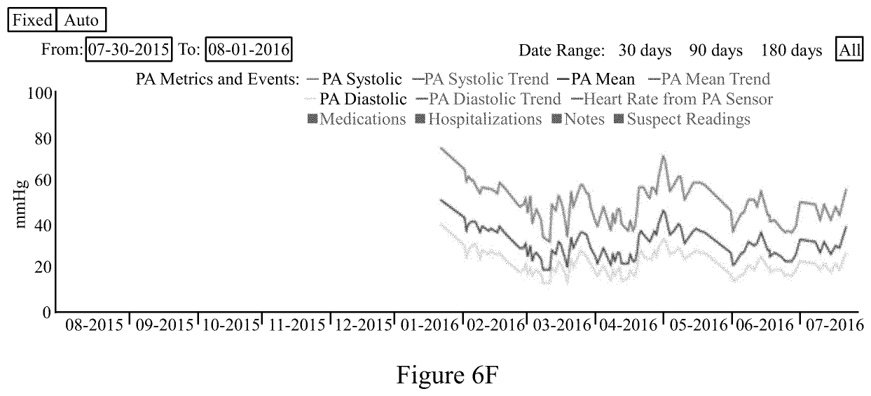

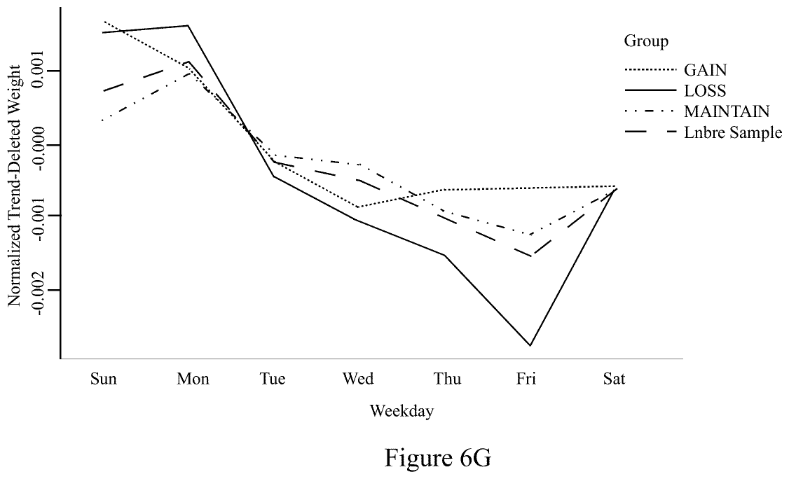

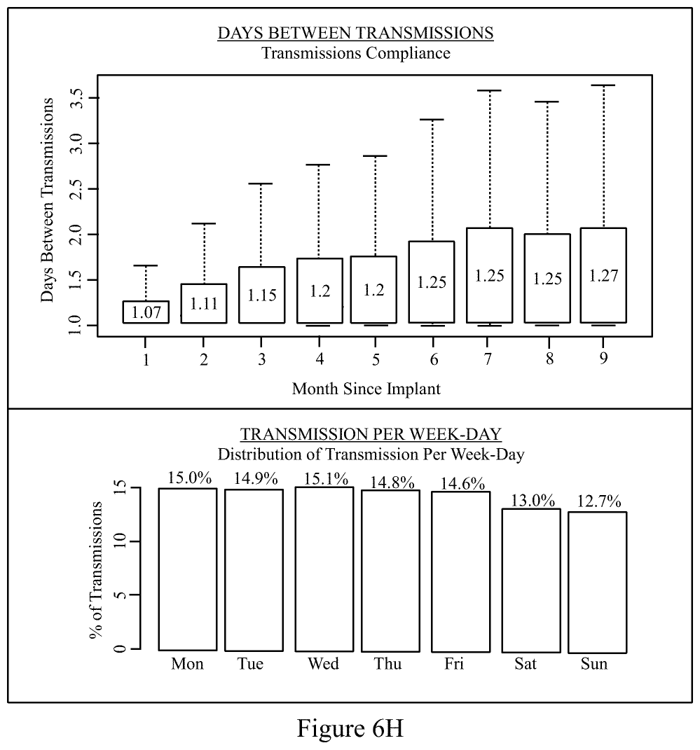

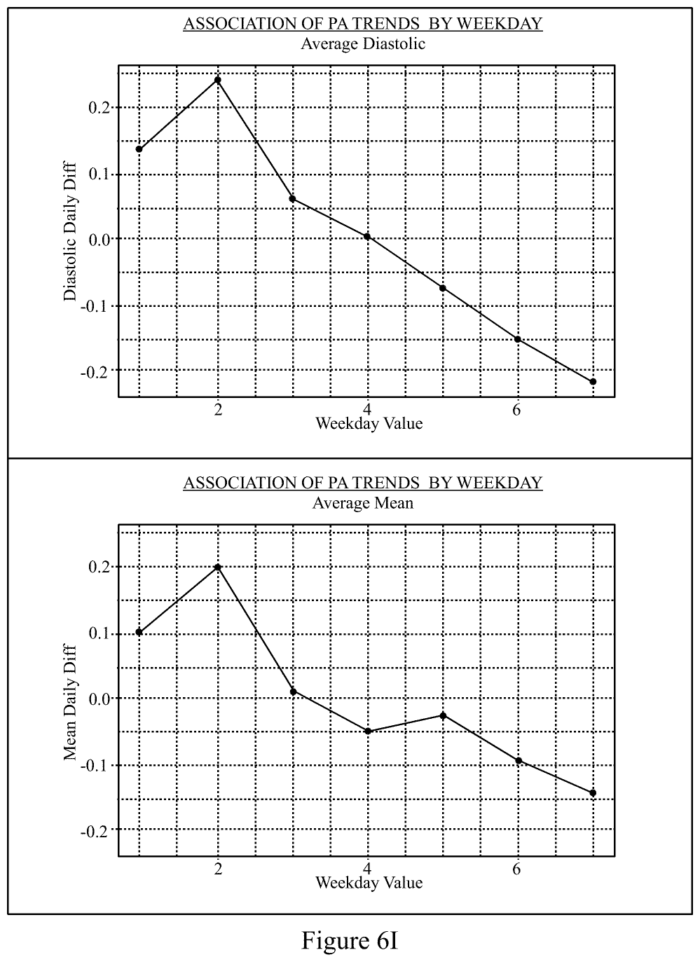

FIGS. 6A-6D illustrate a method for treating chronic disease states based on collection and analysis of IMD data and BGA data in accordance with embodiments herein.

FIG. 6A illustrates a method for collecting new BGA data and based thereon, calculating an BGA Index in accordance with embodiments herein. The method of FIG. 6A may be initiated based on various criteria, such as periodically, continuously, at the direction of a clinician or network manager, in response to receipt of new lab results and the like. Additionally or alternatively, the method of FIG. 6A may be initiated each time a medical network receives a request from a BGA test device to upload new BGA data. For example, each time a BGA test device analyzes a blood sample or other fluid sample from the patient, the BGA test device will send a new data upload request (with the patient ID and test results) to a server, database or other computing device of a medical network. As another example, the BGA test device may wirelessly transmit BGA data to a local external device (e.g., a bedside monitor, laptop computer, smart phone, etc.) and the local external device then sends a new data upload request to the server, database or other computing device of the medical network. The upload request(s) may be utilized as one basis to initiate the method of FIG. 6A. As yet another example, an IMD, PDE device and/or BGA test device may initiate collection of additional IMD, BRM and/or BGA data. For example, an IMD and/or PDE device may track IMD and/or BRM data and detect possible deterioration of patient health. For example, an IMD data trend may cross a threshold, change direction or increase a downward trend. When the IMD detects possible deterioration, the IMD may notify the patient to perform a POC measurement to collect supplemental BGA data. Optionally, when the IMD detects possible deterioration, the IMD may automatically convey a device command (as a treatment notification) to a BGA test device. In response, the BGA test device may automatically collect BGA data. Additionally or alternatively, a PDE device may analyze BRM data to identify when a BRM data trend crosses a threshold, changes direction, or increases a downward trend. The PDE device may identify an extended period of time in which no new BRM data is entered (e.g., an indicator of data entry noncompliance). When the PDE device detects possible deterioration and/or noncompliance, the PDE device may notify the patient to perform a POC measurement to collect supplemental BGA data and/or automatically convey a device command to a BGA test device requesting collection of new BGA data.

Additionally or alternatively, the BGA test device may perform continuous BGA monitoring and locally analyze the BGA data for indications of possible deterioration in a patient health. When the BGA test device identifies a possible deterioration in a patient condition, the BGA test device may automatically convey a device command to an IMD to direct the IMD to collect IMD data.

At 602, a processor determines whether new BGA data has been obtained. When new BGA data is present, flow moves to 604; otherwise, flow moves to 610. Optionally, on a weekly/monthly basis, patients may perform a point-of-care comprehensive blood panel test which is then transmitted to a central medical network. At 604, the processor obtains the BGA data. Optionally, the processor also obtains medication related information from the BGA data, where the medication related information may indicate changes in a patient's prescription regiment.

At 606, the processor calculates a body generated analyte-based index (BGA index) based on the BGA data. Examples are discussed below for different physiologic COIs that may be monitored in connection with different types of BGA Index calculations. For example, an BGA Index may represent a GNRI, CONUT and/or PNI that are calculated based on, among other things, a serum albumin level from the BGA data. As another example, the BGA Index may represent a level of an electrolyte related BGA, such as the GFR, BUN, creatinine level and the like. As another example, the BGA Index may represent levels and/or trends for BNP and/or NT-proBNP levels from the BGA data. As another example, the BGA Index may represent cardiac enzyme levels and/or trends obtained from the BGA data. As another example, the BGA Index may represent an INR level, as calculated from a coagulation related BGA indicative of a blood clotting COI of a patient. Optionally, the processor may classify the BGA Index based on various predetermined criteria.

At 608, the processor updates a patient medical record with the BGA Index and BGA data, as well as any medication information (e.g., a current prescription, a prescription change, compliance with prescribed medication, or the like) and any other test results of interest from the BGA data. Optionally, the processor may update the patient medical record to include historic information as appropriate based on patient history. Thereafter, the method may terminate without further action and/or, flow may move to one or more of the processes described herein for applying an application-specific model in connection with providing a diagnosis and treatment recommendation.

The operations of FIG. 6A may be performed each time a patient undergoes a full or partial blood panel analysis. For example, during routine care of heart failure patients, a comprehensive blood panel is assessed periodically. During the comprehensive blood panel, the lab records contain current measures of the patient serum albumin level. Embodiments herein utilize the serum albumin level recorded in the lab test results to generate the BGA Index. Additionally or alternatively, patients may perform a point-of-care comprehensive blood panel test on a weekly or monthly basis. The BGA data results of the POC blood panel test are then transmitted to the medical network and analyzed.

Optionally, the BGA data may be collected by more than one BGA device. The BGA device(s) may include one or more handheld analyte meters and/or measurement devices and a means for collecting data, preserving data integrity, and uniquely identifying patient data received from multiple sources. For example, provided herein is a means to uniquely identify a patient and the BGA data for the corresponding patient when the data is collected from one or more BGA devices. Providing more than one BGA device allows a patient to use multiple sources to collect BGA data. Accordingly, the system described herein provides a patient with more flexibility, which should encourage better compliance to protocols. Further, by having a way to uniquely identify patient data without requiring a patient to only use one analyte meter. For example, data can be centralized and analysis can be done with more assurance that all of the patient's data is being considered in the analyses. Embodiments herein may implement one or more of the methods and systems as described in U.S. Pat. No. 9,760,679, issuing on Sep. 12, 2017, and titled “Data synchronization between two or more analyte detecting devices in a database”, the complete subject matter of which is incorporated herein by reference.

FIG. 6B illustrates a method for collecting new IMD data and based thereon, calculating an IMD Index in accordance with embodiments herein. The method of FIG. 6B may be initiated based on various criteria, such as periodically, continuously, at the direction of a clinician or network manager, in response to communications with an IMD and the like. Additionally or alternatively, the method of FIG. 6B may be initiated each time a medical network receives a request from a IMD device or local external device to upload new BGA data. For example, the IMD collects IMD data continuously, periodically or in connection with select cardiac episodes. For example, when the IMD represents a PAP sensor, the PAP sensor will transmit PAP data to a local external device when the local external device is held against the patient proximate the PAP sensor and delivers power wirelessly to the PAP sensor. Additionally or alternatively, the IMD may represent an active IMD, such as a pacemaker, ICD, subcutaneous IMD, leadless IMD, ICM and the like, that collect cardiac activity signals (near field and/or far field), heart sound signals, impedance signals and the like. The active IMD may upload data nightly, weekly, on demand, in response to the detection of deteriorating patient health, or on another schedule to a local external device. The local external device will send a new data upload request (with the patient ID and IMD data) to a server, database or other computing device of a medical network. The upload request(s) may be utilized as one basis to initiate the method of FIG. 6B.

As yet another example, a PDE device and/or BGA test device may initiate collection of additional IMD data. For example, a BGA test device and/or PDE device may track BGA and/or BRM data and detect possible deterioration of patient health. For example, the BGA data trend may cross a threshold, change direction or increase a downward trend. When the BGA test device detects possible deterioration, the BGA test device may notify the patient to perform an IMD measurement to collect supplemental IMD data. Optionally, when the BGA test device detects possible deterioration, the BGA test device may automatically convey a device command (as a treatment notification) to the IMD. In response, the IMD may automatically collect IMD data. Additionally or alternatively, a PDE device may analyze BRM data to identify when a BRM data trend crosses a threshold, changes direction, or increases a downward trend. The PDE device may identify an extended period of time in which no new BRM data is entered (e.g., an indicator of transmission noncompliance). When the PDE device detects possible deterioration and/or noncompliance, the PDE device may notify the patient to perform a measurement to collect supplemental IMD data and/or automatically convey a device command to the IMD requesting collection of new IMD data.

At 610, the processor determines whether new IMD data is available. When new IMD data is available, flow moves to 612; otherwise, flow moves to 618. At 612, the processor obtains the new IMD data. At 614, the processor calculates an IMD based index (IMD Index) based on the new IMD data. Examples are discussed below for different physiologic COI that may be monitored in connection with different types of IMD Index calculations. For example, an IMD Index may represent a PAP systolic level, PAP diastolic level, PAP mean and the like. As another example, the IMD Index may represent a diuretic response profile that is calculated based on hemodynamic data (from the IMD data) and diuretic medication information. As another example, the IMD Index may represent PAP levels and/or trends derived from the IMD data. As another example, the IMD Index may represent ST segment levels and/or ST segment level shifts determined from cardiac activity data within the IMD data. As another example, the IMD Index may represent levels and/or trends in cardiac output, thoracic impedance, cardiogenic impedance, heart sounds and the like. As another example, the IMD Index may represent AF burden calculated based on CA signals indicative of AF episodes. The IMD Index may further represent MCS parameter levels and/or trends. Optionally, the processor may classify the IMD Index based on various predetermined criteria.

At 616, the processor updates the patient medical record with the IMD Index and any other information of interest from the new IMD data, including the raw IMD data. Thereafter, the method may terminate without further action and/or, flow may move to one or more of the processes described herein for applying an application-specific model to provide a diagnosis and treatment recommendation.

FIG. 6C illustrates a method for collecting new BRM data and based thereon, calculating a COI in accordance with embodiments herein. The method of FIG. 6C may be initiated based on various criteria, such as periodically, continuously, at the direction of a clinician or network manager, in response to communications with a PDE device and the like. Additionally or alternatively, the method of FIG. 6C may be initiated each time a medical network receives a request from a PDE device or local external device to upload new BRM data. For example, the PDE device collects BRM data continuously, periodically, in connection with select cardiac episodes. The PDE device will send a new data upload request (with the patient ID and BRM data) to a server, database or other computing device of a medical network. The upload request(s) may be utilized as one basis to initiate the method of FIG. 6C.

As yet another example, an IMD and/or BGA test device may initiate collection of additional IMD, BRM and/or BGA data. For example, an IMD and/or BGA test device may track IMD and/or BGA data and detect possible deterioration of patient health. For example, the IMD and/or BGA data trend may cross a threshold, change direction or increase a downward trend. When the IMD and/or BGA test device detects possible deterioration, the IMD and/or BGA test device may notify the patient to enter BRM data through the PDE device. Optionally, when the IMD and/or BGA test device detects possible deterioration, the IMD and/or BGA test device may automatically convey a device command (as a treatment notification) to a PDE device. In response, the PDE device may automatically collect BRM data.

At 620, the processor determines whether new BRM data is available. When new BRM data is available, flow moves to 622; otherwise, flow moves to 628. At 622, the processor obtains the new BRM data. At 624, the processor calculates a COI based on the new BRM data. Examples are discussed below for different physiologic COI that may be monitored in connection with different types of BRM data calculations. For example, the BRM data may track a sugar content of a patient's diet, with the characteristic of interest representing a sugar intake index. A patient's sugar intake may be analyzed in connection with blood glucose measurements and other BGA data, such as for diabetic patients. As another example, the BRM data may track a patient's exercise, with the characteristic of interest representing a calorie burning index. A patient's daily calorie expenditure may be analyzed in connection with trends in PAP levels (derived from IMD data) and/or electrolyte levels indicative of kidney behavior (derived from BGA data).

Optionally, the operations of FIG. 6C may be implemented when a patient does not actively enter BGA data. For example, a patient treatment/monitoring program may call for a patient to enter certain BRM data periodically (e.g., answering certain health, diet and/or exercise related questions daily or weekly). When a patient does not enter BRM data at the prescribed times or on a periodic basis, the operations of FIG. 6C may record the non-entry of BRM data as “noncompliant BRM data.” Additionally or alternatively, indirect BRM data may be derived from IMD data and/or BGA data. For example, select PAP trends may be indicative of certain patient behavior (e.g., weekend diet/exercise patterns). The fact that the patient is not transmitting direct BRM data at predefined periods indicates non-compliance. The fact that the PAP increases over the weekend may indicate high sodium/high calorie diet. Optionally, when an increase in the PAP trend is identified over a weekend, a potential in the increase of sodium/high calorie foods may be verified utilizing geo-location information for the patient (e.g., the patient went to a fast food restaurant), utilizing nutrition tracking application operating on a portable device and the like. When the select PAP trends are identified (with or without confirmation from geolocation information and nutrition tracking applications), the trend may be treated as indirect BRM data that is utilized to calculate a COI and stored in the patient medical record in accordance with the operations of FIG. 6C.

Optionally, the BRM data may be obtained from a third-party application and/or database. For example, numerous applications are implemented on smart phones and other user devices in connection with tracking diets and activity levels. Nonlimiting examples of diet/nutrition tracking applications include “FATSECRET CALORIE COUNTER”, “FOODUCATE”, “LOSE IT!”, “LOSE WEIGHT WITHOUT DIETING” applications, and the like. Nonlimiting examples of activity tracking applications include the FITBIT application. In accordance with embodiments herein, the information collected by third-party diet and activity tracking applications may be imported into a BRM data entry application operating on the same PDE or a different electronic device. Optionally, the information collected by the third-party diet and activity tracking applications may be conveyed from the electronic device to a server implemented in connection with embodiments herein. As a further example, the third-party diet and activity tracking application may convey the diet and activity information directly to a remote server implemented in connection there with separate from the BRM data entry application operating on the PDE. The remote server may then convey the diet and activity information to the PDE device and/or server utilized in connection with embodiments herein to collect, track, analyze and store BRM data.

As a further example, embodiments for the ASM may utilize BGA and/or IMD data as indirect indicators of BRM data. For example, the ASM may identify the fact that the patient is not transmitting BRM data at predefined periods, thereby indicates non-compliance. When the ASM does not receive direct BRM data, one or more other sources may be utilized as indirect BRM data. For example, when the IMD data includes PAP measurements, the ASM may be tracking the PAP trend (e.g., in connection with medical applications described herein). The ASM may utilize the PAP trend as indirect BRM data. For example, the ASM may determine that the PAP increases over a weekend and interpret the increasing PAP trend over certain times of week as an indicator that the patient has been following a high sodium/high calorie diet. Optionally, the determination that the patient is experiencing a high sodium/calorie diet can be verified using geo-location of the patient. For example, the ASM may collect geo-location information from one or more electronic devices carried or otherwise controlled by the patient. A patient may utilize a wearable device (e.g., smart phone, tablet device, fit bit device, smart watch) that tracks a patient's location and periodically conveys geolocation information to a local patient monitoring device and/or remote server. The ASM may compare the patient's location to known locations of various types of establishments, such as fast food restaurants, ice cream shops and the like, and based thereon derive indirect BRM data. The ASM may use the geolocation information as a confirmation when an increase in the PAP trend is identified over a short period of time and during certain days of the week (e.g., a weekend). Additionally or alternatively, the ASM may collect and analyze the geolocation information in parallel with or independent of PAP trend, and derive indirect to BRM data from the geolocation information independent of BGA and/or IMD data. For example, a patient's diet and/or exercise history may be preprogrammed based on a series of questions answered by the patient. Optionally, the patient's diet and/or exercise history may be collected over a period of time, such as through an application operating on an electronic device carried by the patient (e.g., a location tracking application). The ASM may include a geographic map with nodes for restaurants, gyms, and other establishments of interest. For example, the ASM may utilize the Google map database, or various other mapping applications and databases.

Optionally, the indirect BRM data may be derived from a food ordering application operating on a PDE device. For example, when a patient utilizes a smart phone to order carryout or delivery pizza, Chinese food, and the like, information regarding the food order may be conveyed to the ASM. The ASM may derive indirect BRM data from the online food order. In a more simple example, when the online food order includes a quantity of food associated with an individual consumer, the ASM may attribute all of the ordered food items to the patient. Alternatively, when the online food order includes a quantity of food associated with more than one consumer, the ASM may apportion a select quantity of the online food order to the patient's diet in various manners. For example, when the ASM is aware of a patient's “favorite foods”, if the online food order includes the patient's favored foods, the ASM may identify the corresponding portion of the order and attribute the corresponding portion of the order to the patient's diet. Alternatively, when the online food order is for multiple people, but the patient's food preferences are not known/used, the ASM may assign a portion of the overall order to the patient (e.g., when an order is for four people, the ASM may assign one fourth of each food item to a patient's diet consumption record).

The foregoing examples regarding geolocation information and online food ordering applications are examples of how indirect BRM data may be collected. Optionally, the patient may directly enter BRM data through the use of geolocation information and/or an online food ordering application. For example, when a patient goes to a fast food restaurant, the patient may designate the location (e.g., through dropping a geo-pin on a location tracking application, mark the restaurant through a diet tracking application, etc.). As another example, when a patient uses an online food ordering application, the patient may designate the food items that the patient plans to consume. The patient may designate the food items directly through the online food ordering application and/or through a separate diet/nutrition tracking application.

At 626, the processor updates the patient medical record with the COI and any other information of interest from the new BRM data, including the raw BRM data. Thereafter, the method may terminate without further action and/or, flow may move to one or more of the processes described herein for applying an application-specific model to provide a diagnosis and treatment recommendation.

FIG. 6D illustrates a method for generating a treatment diagnosis and treatment recommendation based on real time BGA data and IMD data in accordance with embodiments herein. The method of FIG. 6D may be initiated based on various criteria, such as periodically, continuously, at the direction of a clinician, network manager or patient, in response to updates in a patient's BGA and/or IMD data (e.g., based on the methods of FIGS. 6A and 6B) and the like.

At 630, the processor accesses one or more patient medical records. The patient(s) medical record to be analyzed maybe identified in various manners. For example, when flow moves from FIG. 6A and/or FIG. 6B to FIG. 6D, the process of FIG. 6D may be implemented in connection with the patient for which new IMD data and/or new BGA data is received. Additionally or alternatively, a physician or patient record manager may designate a patient medical record to be analyzed, such as in connection with an office visit, a telephone conversation, electronic mail and the like. Additionally or alternatively, the process of FIG. 6D may be automatically initiated on a periodic basis for all patients and/or for patients who exhibit certain criteria. For example, the patient medical records, for patients known to have a certain health condition, may be analyzed daily, weekly, monthly and the like.

At 632, the processor correlates a data entry time associated with collection of the medication related information, the IMD data, BRM data and BGA data. The processor analyzes the medication related information, the IMD data, BRM data and BGA data in connection with one or more application specific models (ASM) to generate a treatment diagnosis and calculate a health risk index related to the treatment diagnosis (e.g., at 422 in FIG. 4). The ASM may be implemented in various manners, including but not limited to threshold-based algorithms, template correlation algorithms, lookup tables, decision trees, machine learning algorithms and the like. Various embodiments are described for analyzing different combinations of IMD (from passive and active IMDs), BRM and BGA data in connection select medical applications. Various examples are also provided for treatment diagnosis that may be generated based on the analysis. The ASM analyzes data points from dissimilar data sources (e.g., from the IMD, BRM and BGA data) relative to one another to generate the treatment diagnosis. The data points from the IMD, BRM and BGA data have a relative level of importance with respect to one another, that varies, in connection with calculating the health risk index and generating the treatment diagnosis. The relative level of importance may vary in the context of a particular disease state or health risk index of interest.

In connection with generating a treatment diagnosis, the processor also assigns a health risk index that is indicative of a state or condition exhibited by the patient. Based on the type of medical application, the state of the patient may vary, as will the risk associated with the patient's state. As explained herein, the health risk index may be assigned a value along a scale, a value corresponding to a particular state or condition of the patient, and the like.

At 634, the processor determines whether the health risk index exceeds a threshold. For example, when the health risk index is assigned values along a scale (e.g., 1-10), the threshold may be set at 6. Optionally, the determination at 634 may be based on a relative change in the health risk index. For example, the health risk index would be determined to exceed the threshold when the health risk index is increased by 2 on the scale of 1-10, or when the health risk index is downgraded from moderate to severe. When the health risk index exceeds the threshold, the process interprets the condition as warranting a treatment notification, and accordingly, flow moves to 636. When the health risk index does not exceed the threshold, the process interprets the condition to mean that the patient is maintaining a previous health state which does not warrant a new notification, and accordingly, flow moves to the end of the method of FIG. 6D.

At 636, the processor identifies a treatment diagnosis and treatment notification to be provided in connection with the health risk index. As one example, current IMD data may indicate that a patient's PAP level has increased (relative to prior PAP data). If the PAP levels were considered alone, a potential or candidate treatment diagnosis and recommendation would be to change the patient's diuretic medication (e.g., increase the dosage or change the type of diuretic if the patient is exhibiting a resistance to a particular diuretic). However, in the present example, current BGA data is also obtained and analyzed contemporaneous in time with the current IMD data. The current BGA data may indicate that a blood glucose level is high, and a patient's diabetes may be in an uncontrolled state. The ASM analyzes the blood glucose level information in combination with the PAP information. Given the increase in the blood glucose level, the ASM may determine that it is not preferable to change the diuretic prescription at this time. Instead, the ASM generates a diagnosis and treatment recommendation that first treats the blood glucose level. Once the blood glucose level has returned to an acceptable range, the ASM may then render the diagnosis and treatment recommendation that treats the elevated PAP level. In the present example, the ASM recognized to afford a lower weight (or degree of importance), in the decision-making process, to the elevated PAP level, given that the patient was also experiencing an unduly high blood glucose level.

By way of example, prior to rendering a diagnosis and treatment recommendation, the ASM may determine that additional information is warranted. For example, the ASM may determine that updates or re-measurements of one or more IMD and/or BGA parameters is warranted. In the event that additional IMD data is needed, the ASM may convey a device command to the IMD to obtain and return the additional desired IMD data. Additionally or alternatively, the ASM may desire additional BGA data. For example, the ASM may provide an instruction to the patient to remeasure the PAP, if the patient has not already performed a remeasurement. Once the additional information is collected, the ASM may complete the analysis of the original and additional IMD, BGA and/or BRM data. In the event that the PAP level remains elevated, the ASM may render a diagnosis and treatment recommendation to treat the elevated PAP level.

In accordance with at least some embodiments, one assumption is that the various sources of information allow for remote monitoring to enable decision-making without any active input from the patient. Optionally, a portable device (e.g., smartphone, tablet, PDE device) may implement an application thereon that is configured to ask a patient about medication compliance, Quality of life or specific stimulations in the person's life that, are not related to hemodynamics or physiological state, but might otherwise explain a current health risk Index. As a further example, based on the information entered by the patient, embodiments may “over-ride” an otherwise permanent change in a therapy, as a current circumstance is considered a “one-of” situation.

As a further option, embodiments may “override” an otherwise permanent change in a therapy/prescription based on elevated activity. For example, a patient may exhibit a level of activity that is unusually high relative to a preprogrammed activity level and/or a patient's history of activity. When the patient exhibits an unusually high level of activity for a select period of time, the ASM may recognize such behavior and determined that changes in the IMD data, BGA data and/or BRM data may not be indicative of a degradation of the health risk index and thus may not warrant a change in therapy/prescription. For example, when the patient undergoes an unusually high level of activity for an extended period of time, the patient's blood pressure may become unusually elevated, but does not warrant a therapy/prescription change. Additionally or alternatively, when a patient exhibits an unduly high level activity for a select period of time, the ASM may determine that the patient is not prescription compliant (e.g., the patient is not maintaining within a prescribed activity range), and thus may convey a communication to the patient recommending that the patient reduce the activity level.

At 638, the processor communicates the treatment notification to the corresponding one or more recipients and/or devices. For example, the treatment notification may represent an identification of a patient diagnosis and treatment recommendation. The diagnosis and treatment recommendation may be provided to the patient's physician or other medical practitioner. Additionally or alternatively, the diagnosis and treatment recommendation may be provided directly to the patient. Examples of treatment notifications are discussed herein in connection with particular medical applications.

As explained herein, treatment diagnoses and recommendations may relate to changes in prescriptions, changes in parameters of an IMD and the like. Additionally, treatment diagnoses and recommendations may be implemented in connection with digital health/patient application features that are communicated to the patient through smart phone or another electronic device. For example, an application implemented on a smart phone may allow a patient to track calorie, salt and fat intake as BRM data. The calorie, salt and fat intake are combined with patient activity levels, such as measured by an IMD or wearable device. The treatment notification may be provided to the patient and/or a caregiver, with the treatment notification representing educational material and/or feedback regarding negative health consequences of a patient's lifestyle choices when the combined data (BRM, IMD and BGA data) indicates trends in a negative direction. Similarly, the treatment notification may provide feedback regarding positive health consequences of a patient's lifestyle choices when the combined data indicates trends in a positive direction. For example, when a patient with an IMD and/or BGA test device (who is diabetic or otherwise sensitive to sugar) consumes too much sugar, a notification may be sent to the patient to inform that the excessive sugar has caused a spike in the patient's glucose level. As another example, when a patient with an IMD and/or BGA test device avoids exercise for a period of time, the notification may inform a patient that the patient's lack of exercise has raised a PAP trend and/or introduced an undue burden on a patient's kidneys.

FIG. 6E illustrates a process for automatically identifying high risk behavior in HF patients in accordance with embodiments herein. The process of FIG. 6E overcomes difficulties of conventional approaches. It has been shown that management of NYHA Class III heart failure based on home transmission of pulmonary artery pressure has significant long-term benefit in lowering hospital admission rates for heart failure. HF management involves decisions and actions from managing physicians, HF clinic nursing staff and other medical personnel, who work closely to achieve desired clinical outcomes. The management of an HF patient is further impacted by the patient's behavioral patterns. For example, aspects of patient behavior that affect progression of HF include, among other things, compliance with medication intake, salt restrictions and activity level and the like. In general, a heart failure clinic manages hundreds and thousands of patients. Due to the volume of patients, it is difficult for the physicians and clinic staff to maintain continuous and complete knowledge of every patient's behavior pattern.

The process of FIG. 6E automatically tracks and identifies patient behavior using IMD data (e.g., PAP levels and PAP trends). The PAP level will change due to various reasons, such as acute changes in response to medication ingestion, chronic changes over 1-2 weeks in response to an impending HF exacerbation. Further, changes in the PAP level may follow short-term trends over certain periods of time (e.g., weekly trends Monday-Sunday). When the PAP level changes in accordance with a short-term trend, the trends are considered behavior-based trends as the trend specifically relates to a behavioral environment (e.g., personal and societal) of a patient. For example, a behavior-based PAP trend may show improvements in PAP levels over the course of a work week (e.g., Monday-Friday), followed by deterioration in PAP levels over the course of a weekend (e.g., Friday-Sunday). As another example, during weekdays or over a portion of the week, the behavior-based PAP trend may be relatively constant or exhibit a first rate of change (e.g., improving/deteriorating at a certain rate). However, during other days of the week or over the weekend, the behavior-based PAP trend may change to no longer be constant and/or to exhibit a different second rate of change. As another example, a behavior-based PAP trend may show improvements in PAP levels on certain (e.g., exercise) days of the week (e.g., days a patient attends a regular exercise class), with deterioration or no improvement in PAP levels on non-exercise days of the week. Short-term behavior-based patterns (e.g., 7-day cycle of a week) may have a strong impact on life that is more environmental and behavioral than biological. For example, days of the week determine sleep patterns, physical activity and eating habits. As another example, when considering the pattern of daily weight changes, higher weights may appear early in the week (Sunday and Monday), but then decrease over a remainder of the work week. As one example of a short-term weight behavior pattern, an increase in weight begins on Saturday and a decrease in weight begin on Tuesday.

In accordance with new and unique aspects herein, it is been found that PAP data may be helpful to identify patients who are candidates for implant of a ventricular assist device (e.g., LV assist device), a transplant, a valve repair procedure (e.g., a MitraClip™ valve repair to correct mitral regurgitation) and the like. Various characteristics and trends in the PAP data may be monitored by clinicians and/or an ASM to identify patho-physiology that may be treated with an advanced therapy such as an LV assist device, transplant, valve repair and the like. Currently, a problem remains in that the current methods subject patients to various subjective clinical measurements that may go unnoticed and result in a lack of identification of patients who are very sick patho-physiologically. For example, pulmonary hypertension may lead to an undesirably large degree of perfusion inpatients who are otherwise viewed to be healthy. Embodiments herein describe methods and systems to identify characteristics of the PAP data indicative of poor patient health as well as indicative of candidates for certain types of implants. For example, embodiments herein analyze combinations of cardiac activity signals, such as from an ICM, with PAP data for characteristics indicative of unduly large perfusion resulting from pulmonary hypertension. For example, the ASM may analyze the CA signals and PAP signals in search of certain characteristics of interest in heart rate, arrhythmias pressures and the like. The ICM may measure the heart rate over an extended period of time (e.g. several weeks, months or longer). For example, the ASM may track the level of burden associated with the arrhythmia. Arrhythmias exhibiting high burden (e.g. a large number of events or long duration of events) will exhibit a high PAP pressure. As the PAP pressure reduces, the arrhythmia burden is also reduced. Additionally or alternatively, the active IMD, such as the ICM, may measure activity and provide the activity signals as part of the IMD data. Embodiments herein may examine PAP pressure during exercise to identify a reaction of the level of pulmonary hypertension to the level of exercise. For example, while a patient is exercising, and ICM may measure heart rate, while a PAP sensor measures pressure. As another example, an IMD may utilize an activity sensor to measure a level of exercise, in combination with the PAP sensor measuring pressure. The indication of the level of exercise, in combination with the PAP data, may be analyzed manually or with an ASM in search of an unduly large level of perfusion in connection with pulmonary hypertension.

In accordance with new and unique aspects herein, it has been recognized that certain characteristics of PAP waveforms may indicate mitral regurgitation. The presence of mitral regurgitation may represent one reason why a patient does not respond to medication or changes in prescriptions, such as diuretics or changes in a level of diuretics. When a patient is not responding to diuretics, if the PAP waveforms indicate mitral regurgitation, the combination may be utilized to designate the patient as a candidate for a valve procedure, such as a MitraClip™ procedure and the like. Additionally or alternatively, the CA signals and PAP data may be analyzed to identify whether a patient is a candidate for ablation.

In accordance with new and unique aspects herein, it has been recognized that PAP data may be utilized to track an effectiveness of a procedure, such as a MitraClip™ procedure, ablation and the like. For example, when a procedure is implemented to correct mitral regurgitation, a patient's PAP measurements are expected to improve within a predetermined period of time (e.g. within 30 days of implant). Embodiments herein monitor the PAP pressure to track whether improvement occurs when expected. The PAP data may be analyzed for various PAP waveform patterns indicative of continued mitral regurgitation, thereby indicating that the patient is not responding to the procedure. For example, following an implant procedure, the patient's hemodynamics are expected to improve. The improvement is expected to appear within the PAP waveform, such as within the behavior of the dicrotic notch, dp/dt, systolic PAP, mean PAP, variability and the like. When expected improvements are not seen in the PAP waveform characteristics of interest, the condition as interpreted to indicate that the patient is not responding to the procedure. As another example, following an ablation procedure, a patient's PAP data may be measured to determine whether the condition justifying the ablation as returned. For example, the PAP may be measured post procedure for an effectiveness for an extended period of time, such as one to two years.

FIG. 6G illustrates examples of weekly weight gain/loss trends for a patient population. The horizontal axis designates the day of the week, while the vertical axis designates weight along a normalized scale. The various graphs correspond to segments of the patient population that experienced weight gain, weight loss, maintained weight within a desired range and the overall patient population. As shown in FIG. 6G, patients typically experience weight gain from Friday to Monday and weight loss Monday to Friday.

The process of FIG. 6E may be implemented as part of the analysis performed by an ASM when generating treatment diagnosis and recommendations. Additionally or alternatively, the process of FIG. 6E may be utilized as criteria to trigger a more detailed ASM analysis. The operations of FIG. 6E may be utilized to build a metric that may then be used by the ASM to monitor periodic changes in PAP levels/trends and based thereon, render a treatment diagnosis and recommendation for specific clinical actions to arrest HF exacerbation. As explained herein in connection with FIGS. 6A-6C, IMD, BGA and BRM data are collected over time.

At 650, a processor analyzes IMD data for a patient of interest to identify behavior-based patterns (if present) in the IMD data. For example, the IMD data may represent PAP data. Patients who have PAP sensors implanted are supposed to collect PAP data on a predetermined regular schedule at a predetermined time each day. For example, the patient may be directed to measure PAP data in the morning each day prior to ingesting medications. The PAP measurement represents a patient's daily hemodynamic status. FIG. 6F illustrates a chart plotting an example of a collection of PAP measurements and PAP trend that may be obtained over time. FIG. 6F illustrates a HF patient's PAP levels and trends associated with mean, systolic, diastolic PA pressures and heart rate. The analysis at 650 may analyze one or more behavior-based patterns, such as transmission compliance/adherent or a recurrent temporary departure from a normal behavior pattern.

At 652, the processor determines whether the analysis at 650 identified a behavior-based pattern indicative of transmission noncompliance. For example, each time IMD data is collected, the IMD data is paired with a timestamp designating the date and time of data collection. A transmission compliant behavior pattern represents a series of IMD data samples collected at predetermined or even intervals over time. For example, when IMD data samples are collected daily at the same time or within a predetermined window of time each day, the collection of IMD data samples would be considered transmission compliant. Alternatively, sporadic transmission behavior represents IMD data samples collected at uneven points in time, such as when days are skipped. For example, when the series of IMD data includes gaps for one or more days, the series of IMD data exhibits a transmission noncompliant behavior pattern. As another example, a noncompliant/sporadic transmission behavior pattern may be declared when three or more days pass without collecting IMD data. As another example, the transmission noncompliant behavior pattern may be declared when the IMD data samples are collected daily, but the time of day at which the IMD data samples are collected is outside of the predetermined window of time (e.g., too late in the day after the patient has taken prescribed medication). When the IMD data exhibits a transmission noncompliant behavior (e.g., sporadic transmissions), flow branches to 654.

At 654, the processor determines a treatment recommendation that includes an action to be taken in connection with the transmission noncompliant behavior pattern. The nature of the action to be taken may relate to the nature or severity of the transmission noncompliance. For example, when sporadic transmissions are declared (e.g., IMD data has not been collected for three or more days), the process may initiate an automated call to the patient to check on the physical status of the patient. Optionally, the process may provide a treatment recommendation that medical personnel call the patient. Additionally or alternatively, the treatment recommendation may represent a communication to the patient to request that the patient modify their behavior and ensure daily transmission of IMD data. Alternatively, when the transmission noncompliant behavior pattern is less severe (e.g., one late transmission of IMD data, one day of the skipped IMD data), the process may determine that no further action is necessary and no behavior modification is required. As a further example, if the lack of transmission is due to travel or hospitalization, no behavior modification related action is required. At 654, when further action is appropriate, flow moves to 656. At 656, an automated call may be initiated or a recommendation to place a call is provided to medical personnel. Alternatively, at 654, when no action is needed, the process of FIG. 6E ends.

Optionally, further statistics may be recorded in connection with transmission behavior patterns. For example, the transmission compliance/adherence may be characterized using the date of IMD data transmissions. The dates of the IMD data transmissions are then evaluated for each patient to estimate the days between transmissions statistics. For example, the dates of IMD data measurements/transmissions may be reviewed to identify weekday transmission trends.

Optionally, at 654, transmission compliance trend information may be presented to medical personnel. FIG. 6H illustrates examples of transmission compliance trend information that may be displayed and/or recorded. FIG. 6H illustrates two types of information that may be presented, such as “Days between Transmission” and “Transmission per Week-day”. The Days between Transmission graph tracks a number of days between transmissions of PAP data with the horizonal axis indicating the months since implant of the PAP sensor and the vertical axis indicating the days between transmissions. The Distribution of Transmission/weekday graph tracks the day of the week along the horizonal axis and the percentage of transmissions along the vertical axis.

Returning to 652, when the IMD data is determined to be transmission compliant, flow moves to 658. At 658, the processor determines whether the IMD data is indicative of a recurrent temporary departure from a normal behavior pattern. For example, a recurrent temporary departure from a normal behavior pattern may be indicated in a weekly trend in which a PAP level temporarily rises but then returns to a normal trend. For example, the processor may analyze PAP levels over the course of a week to identify upper and lower PAP levels achieved during the interval of interest (e.g., the week). The processor calculates a relative percentage difference in PAP over the week. A relative increase of ×% over the weekend is labeled a normal behavior pattern, whereas a relative increase of 2×% is labeled an abnormal departure from the normal behavior pattern and represents an alarming trend. A relative increase of 2×% would not represent a recurrent temporary departure. When the processor identifies an abnormal departure, flow moves to 662, where one or more actions are taken as discussed hereafter. Alternatively, when the processor identifies a recurrent temporary departure, flow moves to 660.

At 660, the processor generates one or more patient-based treatment notifications associated with a determination of a recurrent temporary departure from the normal behavior pattern. For example, the action may include sending a message to the patient to inform the patient that they are experiencing an increase in PAP each weekend due to their diet (e.g., salt intake) and/or lack of exercise. The message will allow the patient to understand how their weekend diet decisions are perhaps more indulgent than a weekday managed diet. The message may allow the patient to take self-corrective actions, such as reducing salt or taking an extra dose of a diuretic or other course of action. Following 660, the process of FIG. 6E may end as shown at 664. Optionally, flow may continue to 662.

At 662, the processor generates one or more medical personnel treatment notifications. The MP treatment notifications will vary based on whether a recurrent temporary departure was identified. For example, an MP treatment notification may recommend an action for the HF clinic. For example, behaviors that improve a patient's quality of life could be considered by clinicians for HF management. The diuretic dosage for the patient could be set based on the weekly trend in the patient's PAP, where the dosage is tailored to flatten spikes in weekly PAP trends. The treatment notification may also recommend an increase or decrease of the physical activity along with recommendations on medication usage. Additionally or alternatively, the treatment notification may include information that a physician uses to manually render dosage changes. Optionally, the PAP levels and trends may be presented on a visual user interface of the local external device, and/or medical personnel devices for review.

FIG. 6I illustrates examples of PAP trend information that may be displayed to medical personnel and/or analyzed by the processor at 650. FIG. 6I illustrates average diastolic PAP and average mean PAP trends by weekday.

Additionally or alternatively, when flow moves from 658 to 662 in connection with a PAP change this does not repeat and/or continues for to long of time to be classified as temporary (e.g., a long term PAP trend or non-recurrent non-temporary departure). For example, the treatment recommendation may recommend a change in diuretic dosage for the patient that is tailored to lower a long term upward PAP trend. The treatment notification may also recommend an increase or decrease of the physical activity along with recommendations on medication usage. Additionally or alternatively, the treatment notification may include information that a physician uses to manually render dosage changes for long term PAP trends.

In accordance with at least one aspect, the process of FIG. 6E reduces a number of hospitalizations by providing more frequent medication changes and by providing frequent feedback encouraging a patient to modify his/her weekend behavior.

In accordance with aspects herein, methods and systems are provided that track a manner in which patient behavior modulates weekly PAP daily trends, PAP changes and PAP percentage change over a week. A higher modulation in PAP over a week is indicative of poorer PAP control which may lead to worsened health and worse outcomes such as hospitalizations. In accordance with aspects herein, methods and systems are provided that allow physicians to titrate a patient's medication based on their behavior and potentially increase the medication dosage on the weekdays with a preponderance of high PA pressures. In accordance with aspects herein, methods and systems are provided that allow patients to modify their behavior to keep spikes in PAP over weekends in check. The patient may be asked to increase/decrease their physical activity or reducing their salt intake based on their weekly PA pressure profiles and thereby managing PAP.

### Application #1: Chronic Disease and Malnutrition

Next, the discussion turns to embodiments that utilize the methods, devices and medical networks described herein to monitor a chronic disease in connection with malnutrition in connection with early diagnosis and treatment. Malnutrition is common for patients with chronic heart failure (CHF), with the prevalence of malnutrition in chronic HF patients ranging from 16-62%. Even though numerous technological developments are made every year in the field of HF management, malnutrition is often ignored. In general, there is a lack of awareness and consensus on nutritional strategies aimed at improving the quality of life and functional capacity in these patients. In accordance with embodiments herein, data analytics may be utilized to manage micronutrients synergistically with medication-based and device-based therapies for patients with HF. Improving a patient's energetic metabolism and energy transfer affords the potential to improve clinical outcomes in the patient.

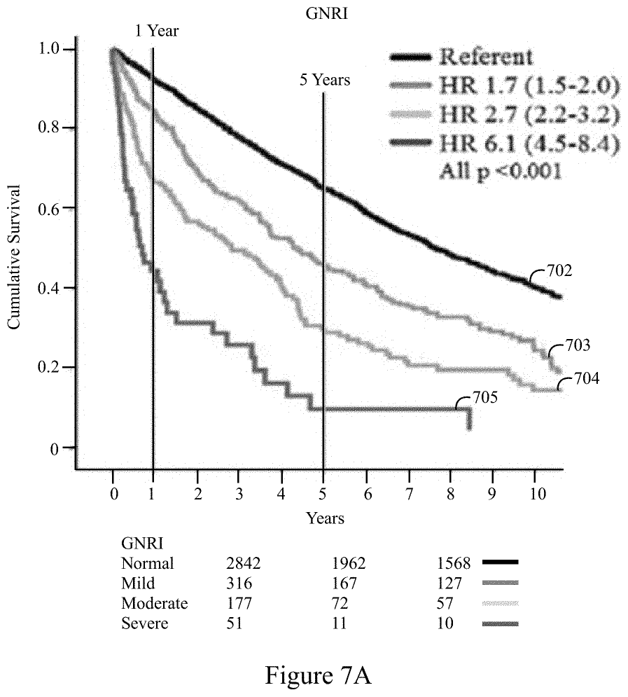

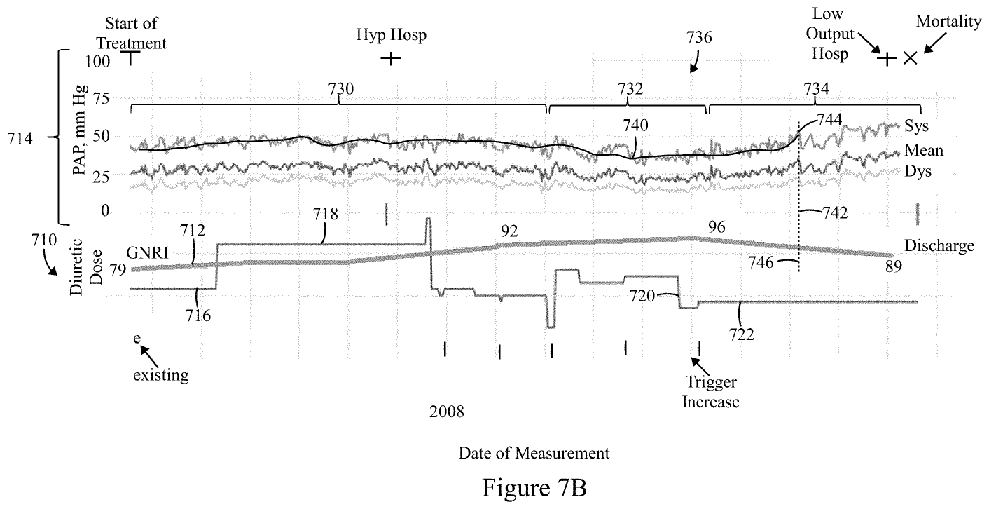

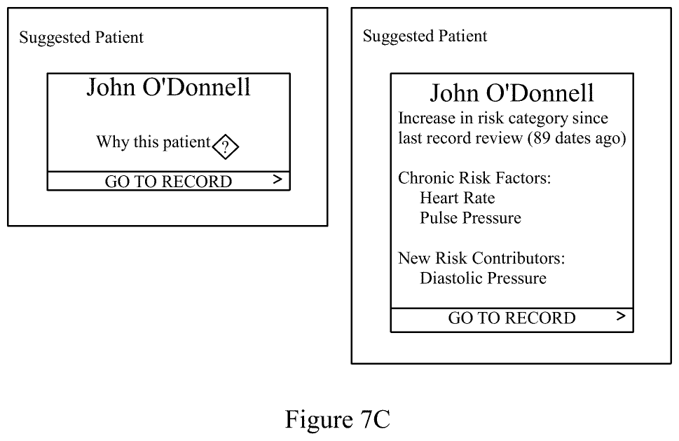

Using data from 2000 patients enrolled in a longitudinal study of patients with CHF (The Hull LifeLab), Sze et al. studied the relation between congestion, malnutrition and clinical outcomes (SZE et al. “Malnutrition, Congestion and Mortality in Ambulatory Patients with Heart Failure” Heart Failure and Cardiomyopathies; 2019). The study demonstrated that concomitant presence of malnutrition and congestion is strongly associated with high mortality. FIG. 7A illustrates example relationships of increased mortality and worsening nutritional status. In FIG. 7A, cumulative survival is plotted along the vertical axis (normalized), while the years of survival are plotted along the horizontal axis. FIG. 7A illustrates graphs 702-705, each of which corresponds to a subset of the patient's that exhibit different hazard ratios (HR) and a corresponding level of malnutrition as quantified by an index, namely the GNRI. The graph 702 corresponds to the patient population that exhibited a normal GNRI and a very low hazard ratio (e.g., HR<1.5). The graph 703 corresponds to the patient population that exhibited a mild GNRI and a corresponding mild hazard ratio (e.g., HR of 1.7, with a variance of 1.5-2.0). The graph 704 corresponds to the patient population that exhibited a moderate GNRI and a corresponding moderate hazard ratio (e.g., HR of 2.7, with a variance of 2.2-3.2). The graph 705 corresponds to the patient population that exhibited a severe GNRI and a corresponding severe hazard ratio (e.g., HR 6.1, with a variance of 4.5-8.4). As indicated by the graph 705, patients who exhibit a GNRI indicative of severe malnutrition, similarly experienced a significantly high risk of death within a relatively short period of time. As indicated by the graphs 704 and 703, patients who exhibited a GNRI indicative of “moderate” and “mild” malnutrition, respectively, experienced slightly lower (but still unduly high risks of death) at one year and thereafter. From the data plotted in FIG. 7A, it can be seen that even a mild level of malnutrition is associated with a significant risk of death at one year with a sustained risk over the years

In accordance with embodiments herein, the methods of FIGS. 6A-6D are applied wherein one physiologic COI corresponds to malnutrition and the BGA Index is calculated as a malnutrition state related index (MSI) based on the BGA data, while another physiologic COI corresponds to congestion and the IMD Index is calculated as a congestion related index (CSI) based on PAP measurements. The “Geriatric Nutritional Risk Index” (GNRI) has been shown to have a desired prognostic value as compared to other such indices. In accordance with embodiments herein, the processor calculates a GNRI, such as using the following formula: [1.489×(serum albumin in grams per liter)]+[41.7×(body weight in kilograms/desired body weight)]. A desired body weight may be calculated using the formula: 22× (height in meters)². The measure of serum albumin represents one of the analytes measured in the BGA data. The processor classifies the present MSI based on the GNRI, thereby taking into account a patient's body weight, height, serum albumin level and the like. For example, the processor may classify the present MSI to be indicative of a normal nutrition level or a malnutrition level that is mild, moderate or severe. For example, various index ranges may be programmed or automatically defined to correspond to different nutrition/malnutrition classification states. For example, a GNRI level>98 may be characterized as a normal nutritional state, a GNRI level between 92-98 may be characterized as mild malnutrition state, a GNRI level between 82-91 may be characterized as moderate malnutrition state, and a GNRI level <82 be characterized as severe malnutrition state.

Optionally, the ASM may utilize one or more sources of information as confirmatory evidence. For example, BRM data may be used as confirmatory evidence, such by having the patient enter self-reported quality of life information from the patient, signs and symptoms indicating fatigue, lack of mobility/exercise and the like.

The operations of FIG. 6A collect, among other things, serum albumin levels (in the BGA data) and calculate an MSI. The operations of FIG. 6B collect, among other things, the PAP measurements and calculate a CSI. The malnutrition state can be categorized using various tools such as GNRI, CONUT, PNI and the like.

Additionally or alternatively, the processor may use other metrics to assess a patient's malnutrition state. For example, the processor may utilize the current BGA data to calculate a Controlling Nutrition Status Index (CONUT) and/or a Prognostic Nutritional Index (PNI). The CONUT metric derives an index based at least in part on a patient's levels for serum albumin, cholesterol and total lymphocyte count. The processor classify patient to have a normal nutrition state with a CONUT score of 0-1 is normal, to exhibit mild malnutrition with a CONUT score of 2-4, to exhibit moderate malnutrition with a CONUT score of 5-8, and to exhibit severe malnutrition with a CONUT score of 9-12. The PNI metric derives an index based on the formula: 10× serum albumin (g/dl)+0.005×total lymphocyte count(mm³). The processor classifies a patient to have a normal nutrition state with a PNI score of greater than 38, to exhibit moderate malnutrition with a PNI score of 35-38 and to exhibit severe malnutrition with a PNI score of less than 35. It is recognized that the foregoing ranges and specific metrics for calculating a malnutrition state related index are merely examples and may be varied, replaced or supplemented.

FIG. 7B illustrates a graphical representation of a portion of the information that may be stored in a patient medical network. In FIG. 7B, time is plotted along a horizontal axis (denoted as “date of measurement”), beginning at a start of treatment time. The patient medical record stores medication related information 710, (e.g., a type of prescription and a dosage level), an MSI 712 and a CSI 714 that are measured and/or calculated over time. The medication related information 710 illustrates that the patient was prescribed an initial dosage level 716, that was then raised to a dosage level 718, with various other increases and decreases over time, until being decreased at a prescription change 720 to a lower dosage level 722. The MSI 712, corresponds to a GNRI that was calculated, based on BGA data, to have an initial level of 79. Over time, measurements of new BGA data resulted in calculations of increases in the MSI to a level of 92, followed by a level of 96. Following the peak in the GNRI level of 96 at 724, the GNRI level decreased to 89. The CSI 714 represents a set of pulmonary arterial pressure (PAP) measurements, namely PAP levels when a heart is at asystole state, PAP levels when the heart is at the diastolic state and a mean PAP level. In the example of FIG. 7B, the systolic, mean and diastolic PAP measurements remained there relatively steady over an interval 730, after which the measurements exhibited a slight dip during an interval 732, followed by a steady increase over an interval 734. Of note, the PAP measurements transitioned from the lower steady-state during interval 732 to begin increasing over interval 734 at a transition point 736. The transition point 736 also generally corresponds to the point in time at which the GNRI level began to decrease from the peak of 96 continuing downward. The transition point 736 also followed shortly after a change in a dosage level of the prescription at prescription change 720.

The method of FIG. 6D analyzes the various information shown in FIG. 7B, utilizing one or more ASM. As a nonlimiting example of a threshold-based approach, the ASM (at 632 in FIG. 6D) may determine whether the MSI exceeds one or more thresholds and/or the MSI transitions from one predetermined range to another. Similarly, the ASM may determine whether the CSI exceeds one or more thresholds and/or the CSI transitions from one predetermined range to another. Further, the ASM may review the medication related information to determine whether a change in a prescription and/or dosage occurred contemporaneous with the new MSI or CSI levels. The ASM assigns (at 632 in FIG. 6D) the health risk index to a low, moderate or severe health risk based on current levels and/or changes in the MSI and CRI, and the medication related information. Optionally, the health risk index may be assigned a value along a scale, such as a numeric scale 1-10, where 10 is indicative of severe health risk and 1 is indicative of low health risk. Optionally, the health risk index may be assigned in alphabetic rating, such as A-F, where an F is indicative of severe health risk and an “A” is indicative of low health risk. Optionally, the health risk index may include multiple indicators, where each indicator corresponds to one of the factors used to derive the health risk index. For example, the health risk index may be formatted with a separate indicator for the MSI, a separate indicator for the CSI and a separate indicator for the medication related information, each of the indicators being adjusted accordingly between low and severe health risks.

The ASM assigns a health risk index based on a combination of i) the relation between the MSI and the corresponding thresholds or ranges, ii) the relation between the CSI and the corresponding thresholds or ranges and iii) current medication related information. For example, as noted herein, the MSI may correspond to a calculation of a GNRI. For example, in FIG. 6D, at 632, the BGA data may indicate that a patient's present malnutrition state corresponds to a GNRI of 90, whereas a prior BGA data measurement may have indicated that the patient's prior malnutrition state corresponded to a GNRI of 95. As one example, a GNRI state between 82-91 may be characterized as moderate malnutrition, and a GNRI state between 92-98 may be characterized as mild malnutrition. Also at 632, the IMD data may indicate a current systolic PAP of 60 mmHg, while a prior IMD data set may have indicated a prior systolic PAP of 48 mm Hg. As a nonlimiting example, a PAP threshold may be set at 50 mm Hg for the current patient, where an increase above the PAP threshold is deemed to represent an unduly high congestion level. The foregoing “negative” changes in the GNRI and systole PAP may occur at a time when no change in a patient's prescription or dosage occurred. When the GNRI and systole PAP both cross corresponding thresholds indicative of degradation in malnutrition and congestion, in combination with no change in a patient's prescription/dosage, at 632, the ASM may assign the health risk index to an “moderate health risk” or a “6” on a scale of 1-10.

Additionally or alternatively, the ASM may compare current and prior levels for the MSI and CRI, to identify a percentage or incremental change. The ASM may further determine whether current levels for one or more of the MSI and/or CSI have substantially changed from a prior level by more than a predetermined incremental threshold (e.g., incremental change, percentage change). The ASM assigns the health risk index based on the change between the current and prior levels for the MSI and/or CRI. Optionally, the ASM may maintain an average or trend for prior MSI calculations and an average or trend for prior CSI calculations. The ASM may compare the averages or trends to the current levels for the MSI and CRI. The ASM may then assign the health risk index based on a difference between the current MSI and the MSI average or trend and based on a difference between the current CSI and the CSI average or trend. For example, with reference to FIG. 7B, an average systolic PAP 740 is illustrated overlaid onto the plot of the systolic PAP measurements. Following the transition time 736, new IMD data and BGA data may be collected. At time 742, a current systolic PAP value 744 is calculated from the new IMD data and a current GNRI level 746 is calculated from the BGA data. The ASM may determine that the current systolic PAP value 744 exceeds the average 740 by more than a predetermined threshold. The ASM may further determine that the current GNRI level 746 indicates a downward trend in the average GNRI. The changes in the GNRI and systolic PAP at time 742, occurred commensurate with a constant prescription/dosage as denoted by 722 and thus are not associated with a prescription/dosage change. Based on the foregoing combination of changes in the systolic PAP and GNRI, and constant prescription/dosage, the ASM may change the health risk index from a low health risk to a severe health risk or change from a 3 to an 8 on a scale of 1-10.

Additionally or alternatively, the ASM may compare a shape of the MSI over time to one or more MSI templates (e.g., based on curve fitting of the trends), and compare a shape of the CSI over time to one or more CSI templates. The templates may correspond to predetermined chronic disease states, for example with a first template exhibiting a morphology of the CSI over time associated with a present patient's baseline behavior (e.g., corresponding to a relatively stable disease state). Optionally, one or more baseline templates may be defined, from a patient population, where the baseline templates correspond to CSI morphologies indicative of relatively stable disease states. Further, one or more baseline templates may be defined, for the patient population, that correspond to CSI morphologies indicative of various levels of progressive disease state (e.g., moderate, severe, etc.). The ASM assigns a health risk index based on the correlation between the shape of a patient MSI and a corresponding MSI baseline and based on the correlation between the shape of the patient CSI and a corresponding CSI baseline.

Optionally, the ASM may apply prediction algorithms to predict future values and/or trends in MSI using MSI past trends, PAP, diet intake (e.g., using historical data from all patients). The prediction may be made by the ASM using a model based approach where important features may be identified using techniques like principle component analysis (PCA), forward-backward selection, matching pursuit, and model structures, such as Markov models, hidden Markov models, regression analysis, SVMs, GBMs, and the like. Optionally, the ASM may render predictions based on ensemble methods that use curve fitting. Based on the predictions, the ASM may identify worsening, stable and improving patients, and assign a health risk index in connection therewith.

At 634 FIG. 6D, the processor determines whether the health risk index exceeds a threshold. For example, when the health risk index is assigned values along a scale (e.g., 1-10), the threshold may be set at 6. Optionally, the determination at 634 may be based on a relative change in the health risk index. For example, the health risk index would be determined to exceed the threshold when the health risk index is increased by 2 on the scale of 1-10, or when the health risk index is downgraded from moderate to severe. When the health risk index exceeds the threshold, the process interprets the condition as warranting a notification, and accordingly, flow moves to 636. When the health risk index does not exceed the threshold, the process interprets the condition to mean that the patient is maintaining a previous health state which does not warrant a new notification, and accordingly, flow moves to the end of the method of FIG. 6D.

At 636, the processor identifies a notification to be provided in connection with the health risk index. At 638, the processor communicates the notification to the corresponding one or more recipients and/or devices. For example, in some circumstances, a diagnosis and treatment recommendation may be to modify a dosage level, in which case, the notification may be provided to the physician or medical practitioner. As another example, the diagnosis and treatment recommendation may be to add or change an amount of an ONS that a patient is taking. As another example, the diagnosis and treatment recommendation may be to begin, change or end certain physical activities, in which case, the notification may be provided to the patient, in addition to the physician or medical practitioner. Other nonlimiting examples of a notification include, in part or in whole, a recommendation to schedule an appointment with a physician, schedule an appointment for additional blood work, perform an additional at home POC blood analysis (e.g., utilizing at home BGA equipment), collect additional BGA and/or IMD data. When a notification includes an action that may be performed by a patient alone, the notification may be communicated directly to the patient.

With reference to the example of FIG. 7B, based on the IMD and BGA data collected at time 742, the ASM may set (at 632 in FIG. 6D) the health risk index to a “severe health risk” or level 8. Accordingly, flow moves through 634 to 426 and 428. The notification may include a communication delivered to a physician informing the physician that the patient's chronic disease state and malnutrition state have been classified as a severe health risk. Additionally or alternatively, the notification to the physician may include the display of the graphical information from FIG. 7B to the physician, along with current and past IMD data (e.g., systolic, mean and diastolic PAP values) and BGA data (e.g., the GNRI value, CONUT value, PNI value) and the patient's present prescription/dosage. When the graphical information from FIG. 7B is displayed, additional emphasis information may be added to highlight certain measurements, indices or other characteristics of particular interest, such as to highlight or enlarge regions of the graphs of particular interest.

Optionally, at the time of interest (e.g., at 742), a pop-up notification window may be presented that highlights one or more insight. FIG. 7C illustrates examples of pop-up notification windows that may be presented in connection with a patient. For example, the insight may provide specific trend-related information indicative of a trend in one or more type of IMD data, BGA data and/or BRM data and their correlation/association. For example, as described herein, the ASM may implement a patient prioritization process. In connection with the patient prioritization process, the ASM may present one or more pop-up notification windows with a patient name and individual variables with one or more arrows pointing up or down. Additionally or alternatively, the notification window may also include an underlying summary regarding BGA and/or IMD data (e.g., “1 mmHg increase in mean PAP increase risk of death by 3%”).

As yet another example, the diagnosis and treatment recommendation may be provided in connection with adding or changing an ONS or other micronutrient. As noted herein, micronutrients may be taken by patients to act as a synergistic support to reduce chronic disease and malnutrition. The micronutrients, among other things, improve energetic metabolism and energy transfer, affording opportunities for improved clinical outcomes. For example, creatine is an important energetic molecule in skeletal muscle in the heart and affords the ability to store and transfer high-energy phosphate. Creatine is synthesized from arginine, glycine and methionine in the kidneys, liver and pancreas. In heart failure patients, the concentration of creatine is reduced, potentially as a consequence of increased sympathetic activity. In accordance with embodiments herein, the BGA test device may measure creatine levels that are reported as part of the BGA data to the medical network. The process of FIGS. 6A-6D may factor in the deficiency in creatine when calculating the MSI. Additionally or alternatively, the creatine level may be recorded directly into the patient medical record. The process of FIG. 6D, when analyzing the MSI and/or BGA data at 632, may identify the deficiency in creatine and, based thereon, include within the notification, a recommendation to increase a patient's intake of a nutritional supplement for the creatine deficiency.

As another example, the notification may recommend a treatment that includes adding or changing a dosage of coenzyme Q10 taken by a patient. coenzyme Q10 is a natural antioxidant synthesized and diet supplied lipid solution cofactor that acts in the mitochondrial membrane. Coenzyme Q10 is present in a relatively high concentration in the mitochondrial electron transport chain, particularly in the synthesis of ATP, in healthy patients, coenzyme Q10 is present in high concentration in the myocardium. The coenzyme Q10 exerts three main biological roles in humans, namely contributing to mitochondrial energy production, stabilization of cell membranes and affords an antioxidant effect. In accordance with embodiments herein, the BGA test device may measure coenzyme Q10 levels that are reported as part of the BGA data. The process of FIGS. 6A-6D may factor in deficiencies of the coenzyme Q10, from the BGA data, when calculating the MSI. The process of FIG. 6, when analyzing the MSI and/or BGA data at 632, may identify the deficiency in the coenzyme Q10 and, based thereon, include within the notification, a recommendation to increase a patient's intake of a nutritional supplement for the coenzyme Q10 deficiency.

As yet another example, the treatment notification may recommend a treatment that includes adding or changing a dosage of vitamin D, iron, Thiamine, Amino Acids (e.g., Taurine, Carnitine, Arginine), carnosine and other micro-nutrient supplements.

As yet another example, the treatment notification may represent a communication conveyed to the patient and/or a caregiver to educate and/or provide feedback to the patient and/or caregiver regarding how present lifestyle choices are inducing negative and/or positive trends in a patient's health. For example, with respect to FIG. 7B, a patient may enter BRM data (e.g., through a smart phone application) indicating that the patient has consumed several food products that are high in salt. The salt content of such food products may adversely impact the intended effect of the prescribed diuretic. Accordingly, a patient's present diuretic prescription may be at a proper level, but a patient may exhibit an upward or downward PAP trend, due to the excessive salt content in the patient's present diet. The treatment notification may include a communication conveyed to the patient smart phone informing the patient that the excessive salt content has raised the patient's PAP trend and the treatment notification may include a recommendation to reduce the salt content of subsequent food products. Optionally, patient behavior may be derived indirectly from transmission non-compliance (e.g., the fact that the patient is not transmitting BRM data at predefined periods) and from PAP trends that are exhibited over select periods of time (e.g., a weekend). For example, when the ASM does not receive direct BRM data, the ASM designates the condition as transmission noncompliant. The ASM tracks the PAP trend with respect to certain periods of time. The ASM may determine that the PAP increases over a weekend and interpret the increasing PAP trend over the weekend as an indicator that the patient has been following a high sodium/high calorie diet.

In accordance with the operations of FIG. 6D, the method analyzes content of patient medical record to identify a patient's present chronic disease state and malnutrition state to identify a patient diagnosis and treatment recommendation. Optionally, the analysis at 632 may include a determination as to whether the patient medical record indicated that the patient has received a prior diagnosis and treatment recommendation from the medical network. When a prior diagnosis and treatment has been provided in accordance with the process of FIG. 6D, the processor determines whether the current diagnosis and treatment represent a change from the prior diagnosis and treatment. When a change is identified, the processor determines and communicates a new notification in connection there with.

While the process of FIG. 6D is described in connection with a particular point in time along the graphs of FIG. 7B, it is recognized that the process of FIG. 6D would be implemented repeatedly or at predetermined times. The process of FIG. 6D would analyze the current and prior medication related information 710, MSI 712 and CSI 714 (FIG. 7B), and determine a corresponding health risk index at multiple points over time. For example, during the interval 730, the process of FIG. 6D may determine that no notifications are warranted. As a further example, at the end of the interval 732, and/or at various points along the interval 734, the processor may determine that notifications are warranted based on changes in the GNRI and PAP systolic, mean and diastolic measurements. For example, at or shortly after the transition time 736, the processor may determine that a patient's malnutrition state has deteriorated, and the congestion level has unduly increased. In connection there with, the processor may develop a diagnosis and treatment recommendation that is conveyed to a physician, medical practitioner and/or the patient. As a nonlimiting example, the notification may recommend to add or increase in ONS, increase the dosage level for the patient's medication, schedule an in clinic visit, add a prescription for a new or alternative medication and the like.

As illustrated in FIG. 7B, utilizing the GNRI, the nutritional status in the patient with chronic disease (CHF) will be continuously or intermittently monitored (e.g., each time the patient's albumin level become available) allowing for early remote diagnosis of malnutrition. Optionally, changes in the PAP level/trend may be utilized as a trigger to collect new BGA data (and obtain a new GNRI). Additionally or alternatively, BRM data may be analyzed and utilized as a trigger to collect new BGA data (and obtain a new GNRI). For example, anemia is an indicator of malnutrition in elderly patients. A portable device may implement an application that is configured monitor a patient's hemoglobin level and based thereon to detect when the patient is experiencing an anemic state. Nonlimiting examples include applications configured to be implemented on portable devices, such as smart phones, to detect an anemic state (e.g., the “HEMAAPP” application developed at the University of Washington, and the smart phone application developed by Wilbur Lam at Aflac Cancer and Blood Disorders Center of Children's Healthcare of Atlanta, and Wallace Coulter, a faculty member in the Department of biomedical engineering at Georgia Tech). When the portable device detects anemia and/or a hemoglobin level of concern, the electronic device may convey a request to a local external device and/or remote server to trigger collection of new BGA data, obtain a new GNR I measurement and/or interpretation of GNRI. The decision at 634 triggers a notification (e.g., prompt) directed to a clinician, such as to prescribe an oral nutritional supplementation (ONS). When lab test results come available or POC measurements are made, the embodiments herein continue to generate serial GNRI levels and determine if the patient is in normal nutritional status. Additionally or alternatively, the process may generate notifications that include outcome data (out-patient clinician visits, in-patient hospitalizations, increase in PA pressure and death) to study how malnutrition results in improved clinical outcomes. CHF patients with malnutrition are more likely to have signs and symptoms of congestion. Accordingly, as the malnutrition is eliminated, the congestion and hence clinical outcomes in the patient should improve. In accordance with the foregoing embodiments, malnutrition severity may be more closely monitored and accounted for. Historically, patients experiencing severe malnutrition experience an increase in the risk of mortality within the first or second year by 2 to 3 times or more, unless actions are taken. The foregoing increased risk of mortality is also experienced in obese patients. Traditionally, when considering obese patients, a nutritional supplement was not necessarily considered as one of the first potential treatments. In accordance with the foregoing operations, embodiments generate diagnoses that include treatment recommendations to add/modify nutritional supplements in connection with other available therapies, in an effort to obviate long-term consequences of chronic disease.

In accordance with embodiments herein, the BGA data (serum albumin) indicative of the malnutrition state may be utilized as an initial trigger in the ASM analysis. A treatment recommendation may include, in part, a change or addition of a nutritional supplement. Knowing that the patient's malnutrition state is being treated (through nutritional supplement), the treatment recommendation (or a subsequent treatment recommendation) may go on to provide for other actions (e.g., changes in other medication, changes in programming parameters of one or more implantable or external medical devices). Following a change in nutritional supplements, when a patient's PAP pressure indicates a positive trend, this may be interpreted as an improvement in the patient's overall condition.

The diagnosis for a particular nutritional supplement may vary. Certain nutritional supplements are designed specifically for patients with chronic heart disease and may be part of a treatment diagnosis and recommendation. In addition to monitoring the malnutrition state, the ASM monitors the PAP. Patients with chronic heart disease are typically prescribed a “fluid restricted” diet to limit an amount of fluid and/or foods that cause the body to retain fluid. Some patients may not follow the fluid restrictions prescribed to them, which allows for an abnormal increase in a volume of blood that collects in the blood vessels, thereby leading to increases in the pulmonary arterial pressure, among other things. Accordingly, embodiments herein may define blood volumes preferred for patients who also receive recommendations for certain nutritional supplements. The PAP data may then be monitored to determine whether the blood volume indicated by the PAP is correlating in the desired manner to the recommended nutritional supplement. Once the patient is treated with a nutritional supplement, in the event that the patient's PAP increases while taking the nutritional supplement, the ASM may detect the increase in PAP during a subsequent analysis of IMD data, and in connection there with provide a second diagnosis and treatment recommendation that includes a diuretic prescription. The diuretic prescription may be varied based on fluctuations in PAP. Existing updates to diuretic information may be obtained from the patient medical record and/or from medical personnel entering medication information as described in connection with embodiments herein.

The foregoing embodiment is described in connection with using, as the IMD data, pulmonary arterial pressure measurements as a basis to calculate the congestion related state index. Additionally or alternatively, the congestion related state index may be calculated wholly or in part based on other types of IMD data. For example, intra-thoracic impedance may be measured by an IMD and utilized as a proxy to calculate congestion. For example, the intra-thoracic impedance may be measured by one or more of the types of IMDs referenced herein and described in the various publications incorporated herein by reference. As another example, the congestion related state index may be derived from information measured by an IMD that is implemented as a left atrial pressure sensor. As another example, the congestion related state index may be derived from information measured by an IMD that measures cardiac output.

The foregoing examples allow for a diagnosis and treatment recommendation to include prescribing nutritional supplements that may otherwise cause the patient to collect fluid (thereby increasing blood volume in the pulmonary arteries. The level of fluid buildup may be further managed for subsequent diagnosis and treatment recommendations that add or adjust diuretic prescriptions in response to changes in the congestion related state index (e.g., as indicated by the PAP, intra-thoracic impedance, LAP, CO measurements and the like).

### Application #2: Management of HF Patients

Next, the discussion turns to embodiments that provide medical management for heart failure patients. Heart failure may be managed by managing a patient's intake of certain drug classes, such as diuretics (loop diuretics, thiazide diuretics), vasodilators (nitrate, hydralazine) and neurohormonal antagonists (ACE inhibitor or ARB, Beta blockers, aldosterone antagonist).

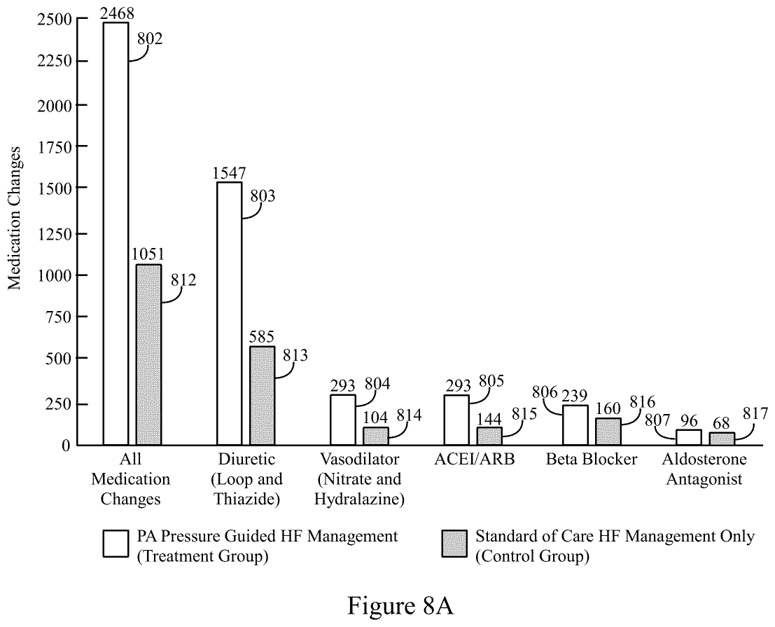

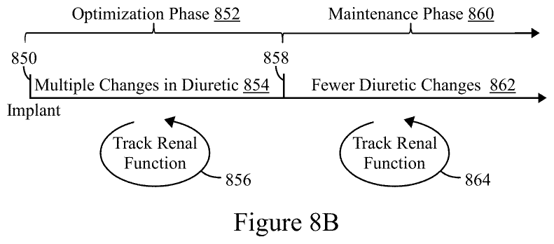

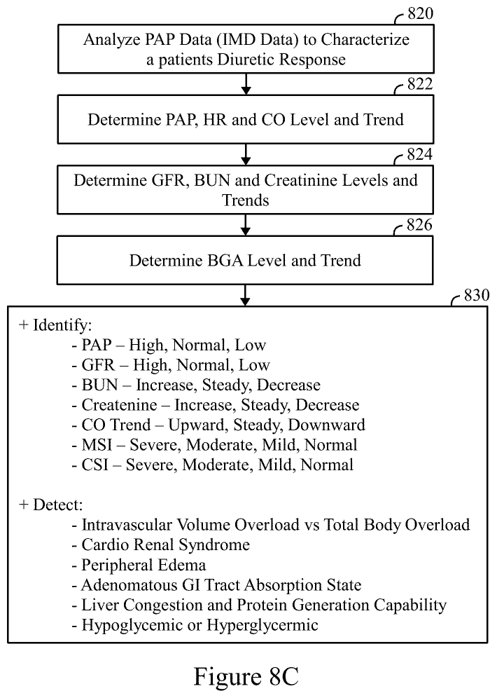

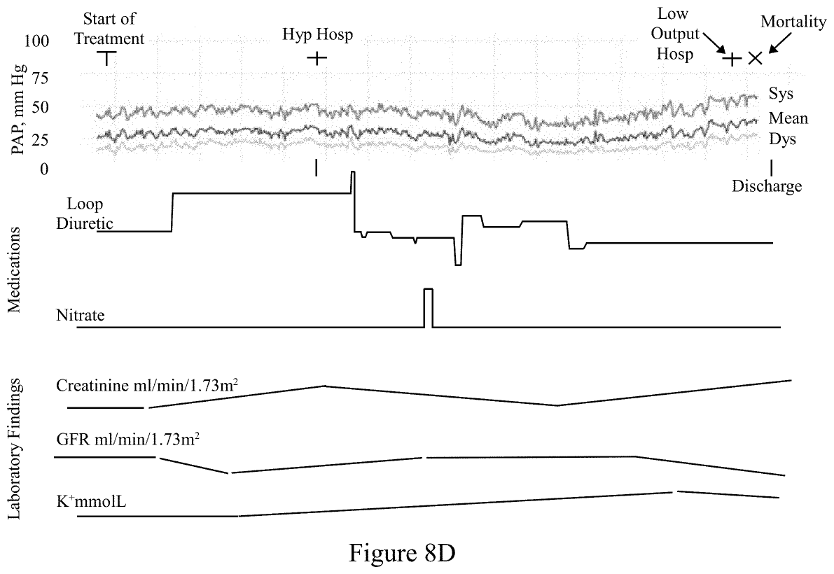

FIG. 8A illustrates a comparison of HF prescription changes for a population of patients during a six-month “follow-up” period. The bar chart 802 represents a count of the total number of medication changes prescribed for the group of the patient population, for whom the medication changes were guided at least in part by pulmonary arterial pressure measurements collected from an implantable IMD (a PAP sensor) (also referred to as the “treatment group” or “actively monitored group”). The bar chart 812 represents a count of the total number of medication changes prescribed for a remaining group of the patient population, for whom medication changes were guided by a standard of care that did not collect and did not take into account pulmonary arterial pressure measurements (also referred to as the “control group” or “blind therapy group”). The treatment or actively monitored group were managed utilizing pulmonary artery pressure guided HF management, which was added to the standard of care management for patients who exhibited certain clinical signs and symptoms. The PAP guided HF management of the medications was done with frequent external measurements, such as GFR (glomerular filtration rate), BUN (blood urea nitrogen), creatinine, and electrolytes. The blind therapy group only received HF management that included standard assessment of weight and patient reported symptoms. The patients received certain changes in drugs over the course of the study.

By way of example, there were approximately 2468 changes in the treatment group patient population medication when the treating medical personnel were provided with PAP measurements on an ongoing basis. In contrast, there were approximately 1061 changes in control group patient population medication when the treating medical personnel utilized the predefined standard of care that did not account for PAP measurements. The bar charts 803-807 represent separate counts of the number of medication changes prescribed for the treatment group in connection with particular classes of drugs, namely diuretics, vasodilators, ACEI/ARBs, beta blockers and aldosterone antagonist, respectively. The bar charts 813-817 represent separate counts of the number of medication changes prescribed for the control group in connection with same classes of drugs, namely diuretics, vasodilators, ACEI/ARBs, beta blockers and aldosterone antagonist, respectively. As a further example, within the treatment group, from the total medication changes of 2468, approximately 1547 of the medication changes related to a change in a diuretic medication. Within the control group, from the 1061 medication changes, approximate 585 of the medication changes related to a change in a diuretic medication. Further, the patient population was analyzed to determine an extent to which the interventions (medication changes) resulted in a decrease in HF hospitalizations in patients managed with and without IMDs that measured pulmonary arterial pressure. The analysis indicated that diuretics were the more frequently modified class of drugs as compared to other classes of drugs taken by patients.

In accordance with embodiments herein, the methods and systems receive the BGA data and medication dosage for each patient. When available, the methods and systems receive IMD data that includes hemodynamics data (e.g., PA pressure from a PA sensor). The methods and systems derive specific clinical insights for managing the patient based on the BGA data and the hemodynamic data. Optionally, medications may be entered manually by the nurse/patient (e.g., Bluetooth via portal into a home monitoring device).

Next, examples are provided for specific clinical insights that may be derived from combinations of patient data collected in connection with management of HF patients.

FIG. 8B illustrates a timeline in connection with an example medical application that utilizes a PAP sensor. The PAP sensor is initially implanted at a site of interest at 850. Patients who receive PAP sensors typically already exhibit relatively high PAP levels. Therefore, when the PAP sensor is initially implanted, the PAP levels measured will likely be relatively high. Following implant, the patient goes through an optimization phase 852 (e.g., for 8-12 weeks following implant), in which medical personnel frequently review PAP measurements and make treatment adjustments 854, such as changing a patient's diuretic prescriptions or other drugs. Optionally, during the optimization phase 852 (e.g., for 8-12 weeks following implant), an automated process may frequently review PAP measurements and render treatment adjustments 854, such as changing a patient's diuretic prescriptions or other drugs. During the optimization phase 852, the medical personnel (and/or processor) make frequent treatment changes to gain control over the PAP level and to reduce the patient's PAP level to a baseline or other desired PAP level (e.g., normal).

Once the PAP level is under control and/or returns to a desired PAP level, the patient management enters a “maintenance phase” 860. In the maintenance phase 860, the PAP system is set/programmed to monitor for changes in the PAP level. For example, the PAP system may track PAP levels to detect when the PAP level varies beyond a threshold tolerance, such as outside a desired range or variation from a baseline level by more than a predetermined amount. The tracking and detecting operations may be performed by the PAP sensor and/or by an external device that communicates with the PAP sensor. As a further example, the PAP system may monitor PAP diastolic pressure with upper and lower limits set. When the patient PAP level exceeds the upper limit or falls below the lower limit, the PAP system notifies medical personnel of the deviation.

In generally, when a PAP level experiences an event outside a tolerance (e.g., exceeds or falls below the upper or lower limit, respectively), the event would trigger a change in a patient's diuretic prescription 862. Medical personnel do not necessarily closely monitor PAP levels for patients who remain within a desired PAP range. When a change in treatment is warranted (due to the PAP level moving outside the desired range), the change is often an adjustment in the diuretic prescription.

The processes described in connection with the Medical Application #2 can be implemented throughout a patient's life, including during either or both of the optimization phase 852 and the maintenance phase 860. The operations of FIGS. 6A-6E and 8, monitor various IMD and BGA data to track and determine how and whether the medication is affecting the patient's body.

The BGA data becomes very important in at least the optimization phase, if not the maintenance phase as well. For example, a patient's diuretic prescription may be adjusted one, two or more times per week during the first few weeks/months. During such frequent diuretic adjustments 854, it is also important to monitor and understand what is happening with respect to other aspects of the patient's physiology. For example, the BGA data may be analyzed at 856 to determine how potassium and creatinine levels are changing because an effect of the diuretic may cause the potassium and/or creatinine levels to become depleted. Embodiments herein allow adjustments to the diuretics (at 854 and 862), while contemporaneously monitoring renal function (at 856 and 864) in a context of PAP levels. The methods and systems determine whether the loop diuretics are having a desired effect, whether nitrates are having a desired effect, and/or whether there is an impending electrolyte abnormality associated with a change in diuretics (e.g., indicating that the kidneys are negatively being impacted by a particular change in fluid retention within the patient due to the prescribed diuretics).

During the maintenance phase 860, medical personnel and/or an automated process do not monitor a patient as closely/frequently as during the optimization phase. However, during the maintenance phase, the processes herein automatically track combinations of the PAP level, electrolyte levels and other BGA data.

FIG. 8C illustrates a method for implementing an application specific model for diagnosis and treatment recommendations for treating HF patients, who exhibit medication resistance (e.g., diuretic resistance), in accordance with embodiments herein. The method of FIG. 8C may be initiated based on various criteria, such as periodically, continuously, at the direction of a clinician, network manager or patient, in response to updates in a patient's BGA and/or IMD data (e.g., based on the methods of FIGS. 6A and 6B) and the like. The method of FIG. 8C represents an embodiment of an implementation for the ASM performed at 632 in FIG. 6D.

At 820, the processor accesses one or more patient medical records. The patient(s) medical record to be analyzed maybe identified in various manners. For example, when flow moves from FIG. 6A and/or FIG. 6B to FIG. 8C, the process of FIG. 6D may be implemented in connection with the patient for which new IMD data and/or new BGA data is received. Additionally or alternatively, a physician or patient record manager may designate a patient medical record to be analyzed, such as in connection with an office visit, a telephone conversation, electronic mail and the like. Additionally or alternatively, the process may be automatically initiated on a periodic basis for all patients and/or for patients who exhibit certain criteria. For example, the patient medical records, for heart failure patients, may be analyzed daily, weekly, monthly and the like.

At 820, the processor analyzes the IMD data, such as PAP data, in connection with prescription information, to characterize a current diuretic response exhibited by the patient. For example, in accordance with embodiments herein, the processor may compare past and present diuretic prescriptions with past and present pulmonary arterial pressure measurements, and past and present diuretic medication information, to determine a diuretic response profile characterization. As nonlimiting examples, the diuretic response profile characterization may be determined in accordance with the systems and methods described in the pending patent application (docket number A16E1036), serial number ______, titled “METHODS AND SYSTEMS FOR DETERMINING DIURETIC RESPONSE PROFILES” and filed on ______, the complete subject matter of which is incorporated by reference herein in its entirety. The diuretic response profile is then used, as explained herein, as a criteria in determining a diagnosis and treatment recommendation for the individual patient. Patients may exhibit a particular diuretic response profile for various reasons. The operations of the method in FIG. 8 analyze additional patient data to determine the particular underlying reason for the diuretic resistance, namely based on patient data indicative of renal function, electrolyte, and nutritional status.

At 822, the processor determines PAP and CO levels and trends based on current and prior IMD data. Each value for a PAP level may represent an individual PAP measurement performed at one point in time. Optionally, each PAP level may be defined as an average of PAP measurements collected over a predetermined period of time (e.g., several minutes, one hour, one day). Similarly, each value for a CO level may represent an individual CO measurement performed at one point in time. Optionally, each CO level may be defined as an average of CO measurements collected over a predetermined period of time (e.g., several minutes, one hour, one day).

Additionally or alternatively, the processor may record the PAP and CO levels over time to define associated PAP and CO trends. Optionally, the PAP and/or CO trends may be defined based on averages for corresponding measurements (e.g., a three-day rolling average, one week rolling average, one month rolling average). Optionally, the PAP and/or CO trends may be defined based on mathematical combinations of ensembles of PAP and CO measurements. For example, each point along a PAP trend may be defined as an average of PAP measurements collected over a predetermined period of time (e.g., several minutes, one hour, one day). Similarly, each point along a CO trend may be defined as an average of CO measurements collected over a predetermined period of time (e.g., several minutes, one hour, one day).

At 824, the processor determines levels for one or more of a patient's electrolyte related BGA data. For example, the processor determines levels for at least one of GFR, BUN, and creatinine, based on the current BGA data, which may correspond to an individual measurement from a BGA test device and/or a mathematical combination of two or more measurements (e.g., an average of test measurements over a few hours, one day, one week). Additionally or alternatively, the processor determines trends for the GFR, BUN and/or creatinine levels based on the current BGA data and prior BGA data and/or prior levels for the respective measurements. For example, each point along a GFR, BUN and/or creatinine trend may be defined as an average of GFR, BUN and/or creatinine measurements collected over a predetermined period of time (e.g., several minutes, one hour, one day).

At 826, the processor obtains the CSI and MSI levels and trends exhibited by the patient. As explained herein, the MSI level and trend may be calculated and updated periodically at 606 in FIG. 6A, while the CSI level and trend may be calculated and updated periodically at 614 in FIG. 6B.

At 830, the processor analyzes the information, obtained and determined in the operations at 820-826, utilizing an application-specific model to assign a health risk index to the patient's present condition. The processor analyzes current and prior medication related information, diuretic response, GFR, BUN, creatinine, BRM data and the patient's overload state utilizing the ASM, and based thereon, a health risk index is assigned. The application specific model may be implemented in various manners, as described herein, including but not limited to lookup tables, decision trees, machine learning algorithms and the like.

Among other things, at 830, the processor identifies whether the PAP and GFR levels are high, normal or low. The processor determines whether the BUN and creatinine levels are increasing, steady or decreasing with respect to prior BUN and creatinine levels and/or prior BUN and creatinine trends. The processor identifies whether the CO trend is upward, steady or downward, and determines whether the MSI and CSI are severe, moderate, mild or normal. The processor may analyze other characteristics or factors related to BGA data and/or IMD data. Additionally, the processor analyzes BRM data that may be relevant in connection with managing an HF patient. For example, the patient's diet and/or exercise regimen (or lack of exercise) may represent a contributing factor in causing a high trend in the PAP, GFR, BUN, creatinine, CO, MSI and CSI, among other things.

At 830, the processor utilizes the determinations regarding the levels and trends in the BGA data and IMD data to detect or declare certain HF characteristics of interest. As nonlimiting examples of HF characteristics of interest, the processor diagnosis/declares whether the patient is experiencing intravascular volume overload or total body overload. The processor diagnosis/declares whether the patient is experiencing cardiorenal syndrome, a peripheral edema, and an adenomatous GI tract absorption state. The processor diagnosis/declares whether the patient is experiencing liver congestion and characterizes a protein generation capability of the liver. The processor diagnosis/declares whether the patient is experiencing a hypoglycemic or hyperglycemic state.

Hereafter, examples are described for various combinations of levels/trends indicated by the BGA and IMD data, along with potential diagnoses that may be rendered by the ASM. For example, the ASM may analyze the relation between a patient's PAP, GFR and diuretic prescription. If a patient has a high PAP and high GFR, the ASM may generate a treatment diagnosis that a more aggressive diuretic prescription (as compared to a current diuretic prescription) could be provided as a treatment therapy. Additionally or alternatively, the ASM may set the health risk index value to a relatively high level indicating a particular state of a patient's HF and/or indicating that a patient's HF state has worsened. On the other hand, the ASM may determine that a patient has high PAP, but a low GFR, in response to which the ASM may generate a treatment diagnosis to use a less aggressive diuretic prescription (as compared to a current diuretic prescription).

An extent to which the ASM calculates an adjustment in a diuretic prescription up or down may be managed in various manners. For example, predetermined increments may be defined for dosage adjustments, such that each iteration through the operations of FIG. 8, a diuretic prescription is increased or decreased no more than the predetermined increment. Optionally, the ASM may calculate an upward or downward adjustment to the diuretic prescription by an amount having a relation to a significance of a change in the patient's HF condition. For example, when the BGA and IMD data indicate that a patient has experienced a relatively minor or slight degradation in an HF state (but sufficient to warrant a prescription change), the ASM may suggest a slight change in a diuretic prescription. Alternatively, when the BGA and IMD data indicate that a patient has experienced a relatively major or severe degradation in their HF state, the ASM may suggest a proportionally substantial change in a diuretic prescription. Optionally, the diuretic response may be considered in connection with a patient's diet. When a patient's diet is preventing a diuretic prescription from having an expected impact, a treatment notification may be generated to inform the patient of the health consequences of how a poor diet is negatively impacting a diuretic prescription. As a further example, when a patient follows a strict diet, limits fluids in a prescribed manner and regularly exercises, the treatment notification may inform the patient that the foregoing lifestyle choices are having a positive effect on the patient's physiology that may result in an option to reduce a diuretic prescription among other things.

Further, the ASM may include, in the analysis, consideration of whether the patient is experiencing “cardiorenal syndrome” or “CRS”. CRS represents a condition in which a therapy configured to relieve congestive symptoms of heart failure is limited by a decline in renal function. The decline in renal function manifests as a reduction in GFR. The cardiorenal syndrome occurs due to high right sided filling pressures and a reduction in the trans-renal pressure gradient from the arterial to the venous side. The cardiorenal syndrome causes a patient's level of BUN and creatinine to go up as if the patient is intravascularly volume depleted, even though the patient is not intravascularly volume depleted. Instead, when a patient experiences a cardiorenal syndrome, elevation in the patient's BUN level and creatinine level are actually indicative of congestion. Patients experiencing cardiorenal syndrome with elevated levels of BUN and creatinine exhibit a somewhat different diuretic resistance scenario. The ASM factors in the foregoing considerations and searches for levels and trends in the foregoing BGA that indicate cardiorenal syndrome, diuretic resistance and the like. Optionally, the ASM may recognize that a decline in renal function is due in part to a patient's lifestyle choices, such as diet, alcohol consumption, sedentary behavior and the like. When a decline in renal function is identified commensurate in time with BRM data indicative of poor diet, alcohol consumption, sedentary behavior, the ASM may determine to postpone a treatment recommendation to change a patient's prescription, and instead send an initial treatment recommendation as a communication to the patient informing the patient that the foregoing lifestyle choices are having a negative impact on the patient's kidneys. As a further example, when a patient follows a strict diet, limits fluids in a prescribed manner and regularly exercises, the treatment notification may inform the patient that the foregoing lifestyle choices are having a positive effect on the patient's renal function that may result in an option to reduce a prescription among other things. Accordingly, the ASM facilitates a collaborative treatment with the clinician and the patient as partners in the patient's care. Additionally or alternatively, the ASM may search for a trend in CO. When the IMD data for the patient exhibits a downward trend in the patient's cardiac output (CO), and the BGA, BRM and IMD data indicate that the patient is exhibiting diuretic resistance, the ASM may diagnose a patient to be experiencing an advanced AF state based on the foregoing combination of conditions.

Additionally or alternatively, the ASM may analyze the MSI to determine a malnutrition state of the patient and to incorporate the malnutrition state into the overall analysis in deriving the prescription. For example, in some patients, a low-albumin level by itself results in peripheral edema. However, after reviewing all of the foregoing data sources, the ASM may determine that it is not preferred to use a physical finding of an edema as a surrogate for increased intravascular volume (increased PAP), as such a surrogate may result in an undesirable diagnosis to increase a prescription for the patient's diuretic medication. Instead, the ASM accounts for the potential that the patient is not experiencing intravascular volume overload, but instead may be experiencing a condition in which the total body is overloaded, but intravascularly. The ASM recognizes the difference between total body overload and intravascular volume overload and avoids a diagnosis that calls for a treatment that is less effective than desired or otherwise.

For example, the ASM may determine that a patient exhibits a normal PAP, but experiences peripheral edema, and exhibits a moderate or severe malnutrition state (as indicated by the MSI) and a low albumin level. Based on the foregoing combination, the ASM may render a diagnosis to replenish the patient in terms of nutrition. The etiology of the foregoing combination of conditions experienced by the patient is multifactorial: decreased ability of the adenomatous GI tract to absorb nutrition and particular proteins; the inability of the liver that is congested results in an inability to build proteins. This can be further impacted by the cognitive ability of patient and their desire to ingest nutritious food, and hence a higher prevalence of malnutrition in these HF patients.

Additionally, the ASM may account for the fact that BUN can be changed by nutritional state. As described in the embodiment for the medical application #1, tracking the nutritional status of the patient allows for correction of malnutrition to avoid un-necessary usage of diuretics. Both medications and dietary intake impact electrolyte levels: and monitoring for potassium levels allow for better titration of the medications.

Diabetes is a very common co-morbidity in HF patients; with over 60% of HF patients having a concomitant diagnosis of diabetes. These patients have a higher risk of fluctuations resulting in episodes of hyper and hypoglycemia. Hypoglycemic events are accompanied by very high changes in PAP, which return to normal when the blood sugar level returns to normal. Exercise does not cause the PAP to rise to the levels associated with hypoglycemic events. The ASM factors in PAP elevations are associated with low blood glucose level, to render a diagnosis recommendation to treat the underling blood glucose management without unnecessarily adjusting the diuretic dosage. The foregoing examples represent non-limiting examples of combinations of the BGA and IMD data that may be analyzed in connection with HF patients.

The present discussion concerns examples primarily related to PAP levels and electrolyte levels. Additionally or alternatively, cardiac output, transthoracic impedance and heart rate may be utilized as inputs at 822. For example, at 822, the processor may obtain the CO level and/or trend, transthoracic impedance level/trend, and/or a heart rate level and/or trend. The analysis at 830 includes analyzing the CO level/trend and/or the heart rate level/trend as part of the generation of the treatment diagnosis and assignment of the health risk index. For example, the analysis at 830 may compare the CO level/trend to a CO threshold, the transthoracic impedance level/trend to a transthoracic impedance threshold, and/or the heart rate level/trend to a HR threshold. Based on the comparison, the analysis at 830 may then go on to analyze electrolyte levels and/or other types of BGA data. Thus, the CO, transthoracic impedance, PAP and heart rate may be utilized as a triggering event to initiate further BGA data collection and/or analysis of the more recent BGA data.

Optionally, the process of FIG. 8D may utilize functional information about the patient, such as activity, fatigue symptoms, quality of life. The functional information may be entered by the patient as BRM data that would enhance the diagnosis. A high PAP level may also be caused by high salt intake, caffeine intake, less/excess fluids, stress and the like. The BRM data may include indicators of high salt intake, caffeine intake, less/excess fluids, stress and the like, which could help the ASM in correcting the diagnosis and treatments recommendations. For example, if a patient enters high salt intake (e.g., using a PDE device), and patient also exhibits a moderately high PAP level, the ASM and/or a clinician may recommend lowering salt intake before starting diuretics.

Following the diagnosis and assignment of a health risk index at 830, flow returns to the process of FIG. 6D and transitions from 632 to 634. At 634, the processor determines whether the health risk index exceeds a threshold. When the health risk index exceeds the health threshold, at 636, the processor generates a treatment notification (e.g., a clinical recommendation) based on the diagnosis rendered at 830. The treatment notification is then conveyed to one or more corresponding recipients and one or more corresponding devices.

The operations described in in connection with FIGS. 8A-8D may be executed in various temporal relations to one another and in various temporal relations to changes in medication. For example, when a patient is prescribed a change in diuretic dosage, the process may wait a select period of time (e.g., 2 days), and then obtain new IMD data (e.g., obtain new PAP measurements 2 days after a diuretic change). The process may then wait another select period of time (e.g., 2 more days), and then obtain new BGA data (e.g., obtain new potassium and creatinine measurements indicative of renal function). Thus, the process obtains a collection of new IMD data and BGA data over select periods of time to monitor a physiologic response to a treatment (e.g., medication change). In the present example, the process affords a real-time analysis, albeit over several days, indicating whether the prescription change caused a positive impact on a primary related physiologic characteristic (e.g., impact of diuretic change on PAP), without introducing a negative impact on a secondary/side effect on another physiologic characteristic (e.g., impact on renal function).

The process described in connection with Medical Application #2 allows the medical personnel to limit an amount of time spent by the medical personnel directly reviewing BGA and IMD data, given that the process automatically preforms the review of the BGA and IMD data. If the process automatically identifies a change in one or more physiologic conditions of interest, based on BGA and/or IMD data, the process generates a treatment diagnosis and recommendation. The treatment diagnosis and recommendation may include a therapy, prescription change and the like that is directly conveyed to the patient. Optionally, the treatment diagnosis and recommendation may include a notification to medical personnel regarding the change in the physiologic condition of interest.

### Application #3: Estimating a Probability of HF Hospitalization

Next, the discussion turns to embodiments that provide predictions for when a condition of an HF patient will deteriorate to a state where the patient should be hospitalized. Embodiments herein provide methods and systems for analyzing any and all available sources of patient data (e.g., IMD data, BGA data, etc.) and performing a risk assessment that the patient will experience a predetermined event, such as a heart failure (HF) episode or other episode warranting hospitalization. The risk assessment includes an assessment of whether the patient will experience the predetermined event within a predetermined period of time (e.g., the next 30 days, 60 days). Additionally or alternatively, embodiments herein may prioritize patients for clinician management. By integrating an analysis of multiple types of data from dis-similar data sources, embodiments afford a more accurate determination of risk assessment.

The ASM analyzes IMD and BGA data to generate a diagnosis and treatment recommendation. In the embodiment of medical application #3, the diagnosis and treatment recommendation include a risk score related to the probability or likelihood that the patient will have an HF episode or other episode that will require hospitalization within a given period of time (e.g., the next week, next month). Optionally, medication changes may be done remotely, instead of at an office visit. When an in-office appointment, or visit is required, an algorithm may be used, based on the risk score and patient specific parameters, to calculate a priority score. The patient-based parameters can include time since a last PAP measurement, medication usage, changes in risk score, changes in medication prescription, and the like. Once the priority score of a patient is determined, the priority score of the patient is compared to other patient risk scores and the patient is prioritized accordingly. As nonlimiting examples, methods and systems may be provided for assigning risk scores to patients in connection with predicting impending hospitalization. As one example, embodiments herein may implement the methods and systems described in the pending patent application (docket number 13464USO1, Ser. No. 16/262,592, titled “SYSTEM AND METHOD FOR PRIORITIZING PATIENTS FOR CLINICAL MANAGEMENT” and filed on 30 Jan. 2019 the complete subject matter of which is incorporated by reference herein in its entirety.

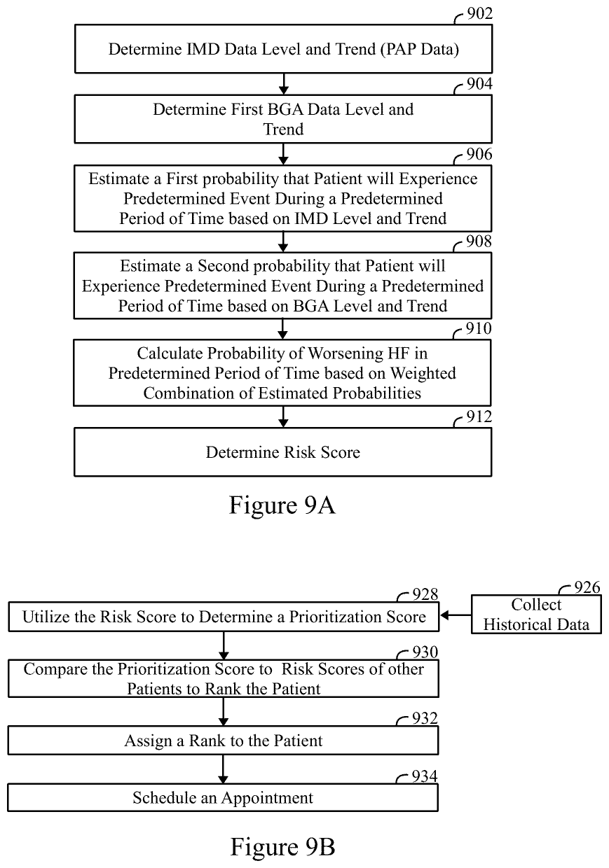

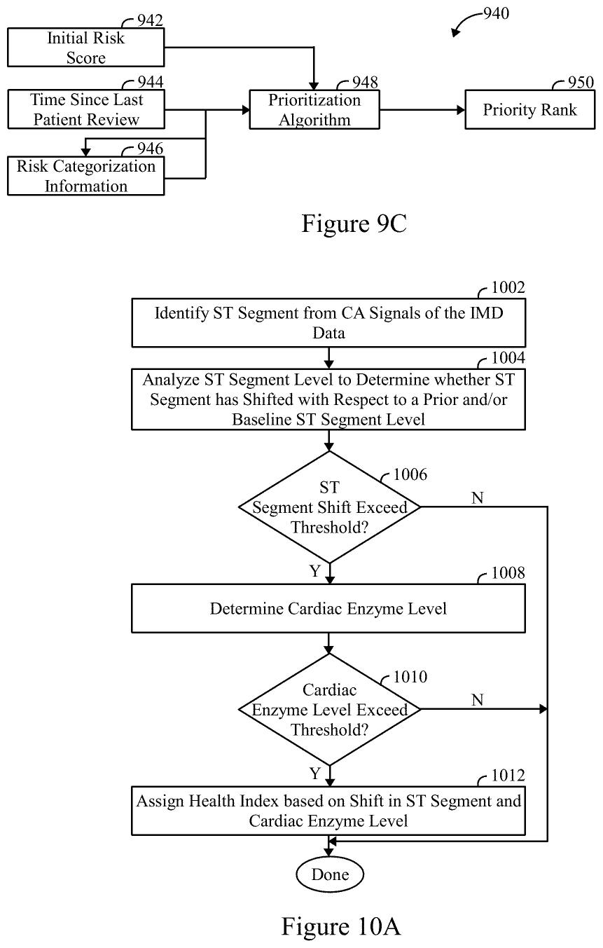

FIG. 9A illustrates a method for determining a risk score for a patient and predicting a risk of an impending hospitalization for the patient. The method of FIG. 9A may be initiated based on various criteria, such as periodically, continuously, at the direction of a clinician, network manager or patient, in response to updates in a patient's BGA and/or IMD data (e.g., based on the methods of the figures herein), in response to negative trends detected by an IMD, BGA test device and/or BRM device, and the like. The method of FIG. 9A represents an embodiment of an implementation for the ASM performed at 632 in FIG. 6D. At 902, the processor accesses one or more patient medical records. The patient(s) medical record to be analyzed maybe identified in various manners. For example, when flow moves from FIG. 6A and/or FIG. 6B to FIG. 6D, the process of FIG. 9A may be implemented in connection with the patient for which new IMD data and/or new BGA data is received. Additionally or alternatively, a physician or patient record manager may designate a patient medical record to be analyzed, such as in connection with an office visit, a telephone conversation, electronic mail, and the like. Additionally or alternatively, the process may be automatically initiated on a periodic basis for all patients and/or for patients who exhibit certain criteria. For example, the patient medical records, for heart failure patients, may be analyzed daily, weekly, monthly and the like.

At 902 and 904, the processor determines levels and trends for desired combinations of one or more types of IMD and BGA data that are indicative of a potential hospitalization. For example, the combination of IMD, BRM and BGA data may relate to frequent causes of hospitalization such as kidney failure, nutrition deficiencies, malnutrition and the like. The following example is primarily in connection with PAP and cardiac markers, although other combinations of IMD and BGA data may be utilized as well. For example, the IMD, BRM and/or BGA data may include markers that are indicative of a potential hospitalization due to pneumonia. For example, the IMD and/or BRM data may include data indicative of activity, transthoracic impedance and the like, that provide trend information for pneumonia and afford a basis for prediction of a potential hospitalization.

At 902, the processor determines a level and trend for one or more types of IMD data that are indicative of a potential hospitalization. For example, the IMD data may represent PAP data. In one example, a sensor detects the PAP and the PAP data is communicated to one or more processors of the healthcare system. In one example, the sensor is in communication with a local external device (e.g., 508 in FIG. 5A) that is remote to the one or more processors of the healthcare system 120. Transmission includes wireless and wire-based transmissions.

At 904, the processor determines a level and/or trend for one or more types of BGA data that are indicative of a potential hospitalization. For example, the BGA data may represent a cardiac marker related BGA (e.g., BNP and/or NT-proBNP) from the BGA data. The level may be based on one sample or based on an average or other mathematical combination of multiple samples. Similarly, a trend may be based on a mathematical function applied to a series of prior levels (e.g., a running average).

At 906, the processor estimates a first (e.g., PAP) probability that the patient will experience a predetermined event (e.g., a heart failure event) during a predetermined period of time (e.g., the next month) based on the PAP level and/or trend. In one example, the first probability is estimated by the processor utilizing a statistical model derived from a database for a patient population. For example, when a current PAP level has a certain value, the database for the patient population may indicate that approximately 10% of the patients with the same PAP level were hospitalized within a predetermined period of time (e.g., the next 30 days, six months, etc.) and based thereon may assign a first probability (e.g., 10% the patient will need to be hospitalized within 60 days). The first probability may be based on numerous variables. Variables may include age, detected PAP, preexisting health conditions including kidney disease, heart disease, cancer, high blood pressure, previous heart attack, medication titration, reaction to medication changes, changes in variables including risk score, and the like. In one example, estimating the first probability includes determining the time since a last appointment and since receiving medication titration data. Additionally and alternatively, the prescription information can include the date when medication was altered and the change in medical usage, or dosage, or type of medication.

Additionally or alternatively, the processor may base the first probability estimate upon historical data related to the probability that the patient will experience the predetermined event during the predetermined period. For example, at 906, the processor collects historical data related to the patient. This includes age, height, weight, medications, changes in medications, previous medical procedures, diagnosed medical conditions, medical history, time since last appointment, family medical history, smoking history, drinking history, drug use history, prior health readings such as blood pressure, PAP data, EKG readings, test results, or the like. The one or more processor may receive such information and data from a local memory, a remote processor, a remote memory, the cloud, or from more than one of the local memory, remote processor, remote memory, the cloud, or the like. The historical data may include previous PAP data of the patient, or a group of patients. Additionally or alternatively, the historical data may include one or previous patient risk estimates, patient co-morbidities, demographics, or medication changes. By using different data and information in estimating the probability the patient will experience a predetermined event, an enhanced evaluation is provided, increasing accuracy and improving patient care. Thus, lives may be prolonged, and costs reduced.

At 908, the processor estimates a second (e.g., BNP) probability that the patient will experience a predetermined event (e.g., a heart failure event) during a predetermined period of time (e.g., the next month) based on the BNP and NT-proBNP levels and trends. In one example, the second probability is estimated by the processor utilizing a statistical model derived from a database for a patient population. For example, when a current BNP level has a certain value, the database for the patient population may indicate that approximately 30% of the patients with similar BNP levels were hospitalized within a predetermined period of time (e.g., the next 30 days, six months, etc.) and based thereon may assign a second probability (e.g., 30% the patient will need to be hospitalized within 60 days). Alternatively, the second probability may be assigned based on predetermined criteria. For example, medical personnel may assign different probabilities in connection with particular ranges of BNP levels and/or particular BNP trends.

At 910, the processor calculates an overall probability of a predetermined event (e.g., worsening HF) within the predetermined period of time based on a weighted combination of the first and second probabilities. For example, the overall probability may represent an overall probability that a patient will experience a heart failure episode within a set period of time. By combining the first and second estimated probabilities, embodiments herein allow for a more accurate prediction of heart failure events based on the integration of BGA data (e.g., BNP, NT-proBNP) with the PAP trends and patient characteristics. In particular, both BNP and PAP represent independent types of data that are derived from dissimilar data sources, but are both good indicators of a condition of HF. When a patient experiences a degradation in an HF state, the BNP and PAP similarly change to reflect the worsened HF state. The processor combines readings from the independent types of data (BNP and PAP) thereby reducing a variance in an estimation for worsening HF. For instance if p, is the probability of worsening HF in a coming month, it may be modeled as p=α₀+α₁PAP+α₂BNP+Σᵢβᵢxᵢ where PAP represents a PAP probability, BNP represents a BNP probability, xᵢ are other covariates such as age, gender and comorbidities. The knowledge of laboratory findings (BGA data) enables embodiments herein to better interpret PAP data excursions to allow for highly specific diagnosis of decompensation and a high risk of a HF hospitalization in the predetermined period of time (e.g., the next 30 days). As a nonlimiting example, the first probability may indicate a 30% likelihood that a patient will need to be hospitalized within 30 days, while the second probability may indicate a 50% likelihood that the patient will need to be hospitalized within the same 30 day period. The foregoing model may be applied to derive an overall probability that the patient will be hospitalized within 30 days.

Optionally, the probability of a worsening HF may be considered in connection with a patient's diet and activity level. When a patient's diet and/or activity level are determined to prevent a prescription from having an expected impact, a treatment notification may be generated to inform the patient of the health consequences of how a poor diet and lack of activity are increasing the patient's probability of worsening heart failure. As a nonlimiting example, the notification may inform the patient that, if the patient continues to have a poor diet and avoid exercise, the patient has a 95% likelihood of being hospitalized in the next 30 days for a heart failure episode. As a further example, when a patient follows a strict diet, limits fluids in a prescribed manner and regularly exercises, the treatment notification may inform the patient that the foregoing lifestyle choices are having a positive effect on the patient's physiology (e.g., “you are doing great with your diet and exercise, and you have reduced your likelihood of having an HF episode by 20%”).

As a further example, if a doctor prescribe exercise as an initial treatment, the BRM data may include patient entered exercise compliance information. When the patient does not enter BRM data corresponding to the exercise compliance, the ASM may render a diagnosis and treatment recommendation that includes a recommendation for the nurse/doctor's office to call the patient and request that patient improve their compliance reporting. Optionally, the call may be an automated notification that is automatically conveyed from a server to a local electronic device proximate or under control of the patient. The automated notification may be a predetermined message, such as “Max please tell us about your exercise history”, “Cate, did you work out today”, “Sophie, our records indicate that you have not worked out this week, is that true?”, “Joe, you were doing so well, but lately we have not heard from you. Please continue to tell us about your exercise”. The automated communication may be designed to make a patient more compliant. A doctor may want to look at patient vitals and or PAP during the exercise, and how the exercise/PAP changes over time which may provide a wealth of additional information.

The patient BRM data may be used by the ASM to calculate a probability of HF hospitalization in the next month. The metric of “probability” may not be meaningful to the patient. Optionally, the metric of probability can be presented as a relative indicator (e.g., 2× higher risk or 5× higher risk) of hospitalization in the next 6 months. Optionally, another metric could be “days alive out of hospital” which would take into account the risk of HF hospitalization, length of stay and risk of mortality.

At 912, the processor determines a risk score for the patient based on the combined BNP and PAP probability estimates. Optionally, the processor may alter the risk score based on patient-based parameters to determine a priority score. In one example, patient parameters include time since a last appointment, visit, or measurement, previous patient risk scores, changes in medication usage, and the like. Patient based parameters may also include data related to a group of patients, such as demographics, percentage of patients with similar test results that experience the event in the predetermined period, effect of medication on similarly situated patients, morphological data, including waveform data of similarly situated patients, and the like. Similarly situated includes patients that have at least one variable in common with a current patient, including approximate age, PAP, BNP, medication, and the like.

At the conclusion of the operations of FIG. 9A, the risk score may be utilized to generate a diagnosis and related health risk index. Optionally, the process may move to FIG. 9B for further analysis.

FIG. 9B illustrates a process for applying a risk score in connection with implementing a treatment diagnoses and recommendations in accordance with embodiments herein. The operations of FIG. 9B may be initiated following the analysis of FIG. 9A, and/or based on other criteria.

At 926, the processor collects historical data related to the patient and other patients. For example, the processor may collect information related to age, height, weight, medications, changes in medications, previous medical procedures, diagnosed medical conditions, medical history, time since last appointment, family medical history, smoking history, drinking history, drug use history, prior health readings such as blood pressure, PAP levels/trends, BNP levels/trends, EKG readings, test results, or the like. The processor may receive such information and data from a local memory, a remote processor, a remote memory, the cloud, or from more than one of the local memory, remote processor, remote memory, the cloud, or the like.

At 928, the processor alters the risk score based on patient-based parameters to determine a priority score. In one example, patient parameters include time since a last appointment, visit, or measurement, previous patient risk scores, changes in medication usage, and the like. Patient based parameters may also include data related to a group of patients, such as demographics, percentage of patients with similar test results that experience the event in the predetermined period, effect of medication on similarly situated patients, morphological data, including waveform data of similarly situated patients, and the like. Similarly situated includes patients that have at least one variable in common with a current patient, including approximate age, PAP, medication, and the like. In the present example, the processor alters the risk score based on patient parameters to arrive at the priority score for an individual patient. Optionally, multiple estimations, calculations, determinations, operations, or algorithms are utilized to alter the risk score and arrive at the priority score. In one example, determining the final patient risk score may include calculating the time since a last appointment, and receiving medication titration data. The medication titration data can include the date when medication was altered and the change in medical usage or dosage or type of medication.

Optionally, the prioritization algorithm may utilize predetermined weights for each of the risk score, time since last appointment, and medication titration data in determining the priority score. In an example the prioritization algorithm is a machine learning algorithm and modifies the weights over time based on patient data related to previous patients that experience the event, such as a heart failure event of a patient compared to their risk score at the time of the event.

At 930, the processor compares the priority score to priority scores of other patients in order to filter or rank the patient compared to other patients. For example, patients that have a priority score corresponding to a “high risk” of an HF episode, may be filtered or ranked at a top of a list for immediate action. Patients that have a priority score corresponding to a “low risk” of an HF episode, may be filtered or ranked at an intermediate or lower point on the list as not needing immediate action.

In one example a comparator is utilized to make the comparison. In another example the priority score of all patients is placed on a list that is in numerical order, and the priority score of the patient inserted in order onto the list. Optionally, the list to visually illustrated on an interface with the priority score of the patient next to the list. In this manner, a clinician or scheduler with knowledge of all of the patients may use the information to schedule the patient based on additional information that cannot be quantified by the clinician management system. Such additional information includes known other appointments, ability of individual patients to get to the facilities for tests on certain days, patient specific work schedules, and the like.

At 932, the processor assigns a rank to the patient based on the comparison of the priority score to the priority scores of the other patients. In an example the rank of the patient is assigned based on an ascending or descending order of a patient on a prioritization list and patient is provided a rank according. Specifically, the patient with the highest score is given a rank of one (1), the patient with the second highest score is given a rank of two (2) and so on. Additionally, in an example where the amount of time since a previous appointment, visit, or measurement is a variable, each patient on the list has their priority score recalculated after a predetermined interval. In one example, the clinician management system automatically updates a patient's score after two days, five days, seven days, ten days, and fourteen days. Optionally, the higher the priority score, the more iterations are provided for updates, thus for a high-risk category patient, their score may be updated daily. Alternatively, every patient on the list is updated periodically, such that updates occur independent of when a patient was placed on the list. Thus, the entire list may update daily, weekly, bi-weekly, and the like.

At 934, the processor schedules an appointment for the patient based on the patient rank. In one example, based on a category a patient is placed based on their priority score determines the starting point for when to schedule an appointment. For example, for an individual that is in the low risk category, the processor begins searching for open appointments that are three weeks away and place the patient in the first available time slot more than three weeks away. Alternatively, for a high-risk patient, the processor searches for open appointments that are two days away and place the patient in the first available time slot more than two days away. Additionally and alternatively, a high risk patient may be given priority, such that a high risk patient must be scheduled within a predetermined period, such as two weeks. In a case when no appointments are available during the two weeks, the processor searches for the patient with the lowest risk score scheduled over the next two weeks, and as long as that patient is not a high risk patient, the one or more processors send a message, through email, text, and the like, and reschedule the scheduled patient to a later time to ensure the high risk patient has priority. If all of the patients over the two-week period are high risk, the one or more processors in one example communicate with remote clinician management systems to find a different location where the patient may be scheduled for an appointment, visit, or measurement over the predetermined two-week time period.

FIG. 9C illustrates a block diagram of an example method utilized by embodiments herein to determine patient-specific priority rank. In one example, the method 940 is performed by a risk score circuitry and prioritization circuitry provided on one or more of the various servers and devices described herein. In another example the method 940 is performed by software, including of a processor of one or more computing devices. At 942, the processor determines determine an initial risk score. In an example, the risk score algorithm determines a probability of a predetermined event and assigns an initial risk score according. In one example the predetermined event is a heart failure (HF) within a predetermined interval of time. The predetermined interval may be one day, one week, two weeks, one month, and the like. In one example, numerous variables are utilized by the risk score algorithm in determining the probability a patient will experience heart failure within the predetermined interval of time. The variables include historical data, pulmonary artery pressure, heart rate, medication usage, and the like. The historical data can include previous patient risk estimates, patient co-morbidities, demographics, medication changes, and the like. Based on these variables the risk score algorithm determines the probability of heart failure within the predetermined interval of time in order to determine the risk score.

In another example, the risk score is determined by the processor by forming a linear model. In an example, the linear model is formed by receiving systolic PAP, diastolic PAP, and heart rate data from an existing group of patients and creating a generalizable estimator based on the received systolic PAP, diastolic PAP, and heart rate data. In one example the generalizable estimator utilizes measured pulmonary artery pressure to form the linear model. In yet another example, the risk score is determined by forming a non-linear model. In one example, PAP data, systolic PAP, diastolic PAP, historical data, and the like is utilized when forming the non-linear model.

At 944, the processor determines an interval since the last appointment or measurement to be inputted into a prioritization algorithm for determining a priority score. In an example, the time since the last recorded appointment or measurement is determined by utilizing historical data recorded in a memory during a previous visit. Alternatively, the time since the last appointment or measurement is inputted into the one or more computing devices 102 by a clinician at the time of the appointment or measurement. The time determined or inputted may be in units of hours, days, weeks, or the like.

At 946, the processor determines risk categorization information, including categorizing the risk score with a risk category algorithm. In one example, the risk category algorithm receives risk scores from numerous patients and ranks them in a list and groups the risk scores into categories based on the score. In one example the risk score is provided on a scale from 0-100 with 0 representing a completely healthy individual with no risk of heart failure in the predetermined interval and 100 representing a patient that has a 100% probability of having heart failure in the predetermined interval. In this example, the risk category algorithm assigns any patient having a risk score between 90-100 as highest risk, any patient having a risk score between 80-90 as higher risk, any patient having a risk score between 70-80 as average risk, any patient having a risk score between 60-70 as low risk, and any patient having a risk score less than 60 has minimal risk. In yet another example the risk category algorithm utilizes a color-coded bar with an indicia pointer. In this example, colors may range from a color such as red, representing the highest risk to blue, representing the lowest risk. The indicia pointer then indicates where on the color-coded bar a particular risk score is positioned. In yet another example, the risk category algorithm utilizes a highlighting function to highlight the risk score of any patient with a risk score exceeding a threshold value. This highlight can include bolding the risk score, providing a background color, placing a symbol such as an asterisk by a name or risk score, and the like. In each example the risk category algorithm organizes or categorizes a risk score for use after a risk score of a patient is determined.

At 948, the processor determines a priority patient ranking based on the information received from the operations at 942-946. In one example, a prioritization algorithm is utilized to make the ranking determinations based on each input. In another example, only one or two of the inputs from 942-946 are utilized. Specifically, the categorizing at 946 may just be utilized as a tool to provide visual information to a clinician. In an example, the prioritization algorithm considers each of the inputs from 942-946 as variables and weights are assigned to each input in determining the priority ranking. In one example, the ranking algorithm is an artificial intelligence, or machine learning type algorithm that continuously receives heart failure data including when a patient in a group of patients experiences a heart failure event during a predetermined interval before an appointment is scheduled. The time interval and event are utilized in addition to time intervals of patients that did not experience a heart failure event and had an appointment. Based on these intervals, the weights provided to each of 302, 304, and 306 may be altered.

In yet another example, at 948, ranking is determined by determining patients that entered into a high-risk category since their last visit and providing greater or increased weight when determining the rankings to those patients. In one example additional points are added to a determined risk score and a priority score is determined and ranked accordingly. In another example a multiplier is provided to a risk score to determine the priority score and final patient ranking. Alternatively, all patients that have entered into the high risk category are placed in a first category or group and ranked based on risk score to determine prioritization and then all other patients, whether in the high risk category or otherwise are placed in a lower priority category or group than the group being ranked based on risk score. In this manner, an individual with a lower risk score, but having just entered into a high risk category receives a higher ranking than a patient with a higher risk score, but has been in the high risk category for a predetermined interval of time, such as at a previous appointment that occurred a week before, two weeks before, a month before, and the like.

At 950, the processor determines the final patient ranking. In an example, once the priority patient ranking at 948 is finalized, each patient is provided with an individual ranking. In one example, a methodology as described in relation to 948 is utilized to determine the final patient ranking. Once the final patient ranking is determined, the one or more processors may schedule appointments based on the final patient rankings. In one example the one or more processors send electronic notifications with open dates provided to patients based on their patient ranking, category placement, and the like. The open dates are determined based on such patient ranking, category placement, and the like. Alternatively, a clinician or scheduling personal utilizes the list in scheduling. In one example, medical data related to the patient is inputted into a prioritization algorithm during an appointment as measurement are being detected and determined. This includes inputted directly and automatically from a measurement device or monitor, or from a clinician utilizing measurement and monitoring devices. Based on these inputs the prioritization algorithm determines the patient specific priority rank prior to the appointment terminating, such that a clinician, or scheduling personnel, can coordinate with the patient at the end of the visit to set a follow up visit based on the data received during the appointment or visit. Therefore, higher risk patients have a quicker turn-around and follow-up, reducing risk for a heart failure event, potentially prolonging the patient's life, and saving costs associated therewith.

### Application #4—Myocardial Ischemia/Infarction Detection

Embodiments herein provide methods and systems for improved specificity of detecting a myocardial ischemia/infarction. Often, when a patient experiences chest pain, the patient may immediately go to an emergency room for evaluation and treatment. During the ER visit, medical personnel may perform various diagnostic tests, one of which includes testing one or more cardiac enzymes, such as a patient's troponin level. Troponin is one example of a cardiac enzyme (a protein) that is released when the heart muscle has been damaged, such as during a heart attack or other myocardial injury. Today, when patients visit an ER with chest pains, the medical personnel may have limited or no information regarding the patient's medical history as related to myocardial ischemia and/or myocardial infarctions.

Typically, when a patient exhibits certain symptoms, the medical personnel seek to quickly determine whether the patient is experiencing an acute coronary syndrome, such as an acute coronary stenosis. When a patient experiences an acute coronary stenosis, the stenosis causes the heart tissue to produce certain types of cardiac enzymes, such as Troponin. Accordingly, an elevated Troponin level may be a good indicator of an acute coronary stenosis. If a patient has an acute coronary stenosis, the patient typically undergoes coronary bypass surgery (e.g., a left side catheterization) to bypass the stenosis. When a left side catherization is necessary the procedure should be performed relatively quickly. However, left side catheterization is an invasive procedure and should only be performed when necessary.

However, elevated Troponin levels may also arise, when a patient experiences other heart episodes, that do not warrant bypass surgery, such as a left side catherization. For example, patients with chronic heart disease experience congestion which “stretches” the heart and places strain on the heart that creates “mis-matches” in blood flow and myocardium. The stretching process, caused by congestion, damages cells of the heart tissue, which causes the heart tissue to produce certain types of cardiac enzymes, such as Troponin. A patient with normal coronary arteries may still have cardiac myopathy which causes congestive heart failure and elevated Troponin levels. While the patient's Troponin level may indicate a potential acute myocardial infarction, in actuality the patient may not need bypass surgery. If considered alone, the elevated Troponin level would lead to a diagnosis of acute myocardial infarction (albeit incorrect).

Embodiments herein overcome the foregoing problem by automatically combining an analysis of patient data from dis-similar sources, such as combining analysis of IMD data with the analysis of the cardiac enzymes. The analysis of the combined patient data from dissimilar sources allows methods and systems herein to avoid in-correct diagnosis of an acute coronary syndrome (that would lead to acute intervention, such as bypass surgery) when a patient may be experiencing a less severe syndrome (e.g., myocardial ischemia) that leads to congestion.

In accordance with embodiments herein, a healthcare system and method are provided that provide improved specificity of myocardial ischemia detection by tracking ST segment levels, heart rate information and dynamic cardiac enzyme levels. In accordance with embodiments herein, when a patient makes a trip to an emergency room, the healthcare system has already collected sufficient IMD and BGA data to allow medical personnel to accurately and quickly diagnose acute ischemia and/or an acute myocardial infarction with a high degree of specificity.

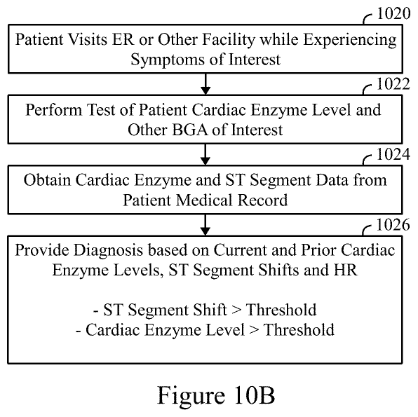

FIG. 10A illustrates a method for tracking certain types of IMD data and BGA data indicative of myocardial ischemia and/or myocardial infarctions. The method of FIG. 10A may be initiated based on the various criteria discussed herein. As described in connection with other embodiments, the processor accesses one or more patient medical records. The patient(s) medical record to be analyzed maybe identified in various manners. For example, when flow moves from FIG. 6A and/or FIG. 6B to FIG. 6D, the process of FIG. 10 may be implemented in connection with the patient for which new IMD data and/or new BGA data is received. For example, when a patient experiences chest-pain, he/she may unilaterally decide to take a troponin test, such as using various types of BGA devices. The BGA data includes troponin I and T levels and is transmitted to the healthcare system. Additionally or alternatively, a physician or patient record manager may designate a patient medical record to be analyzed, such as in connection with an office visit, a telephone conversation, electronic mail and the like. Additionally or alternatively, the process may be automatically initiated on a periodic basis for all patients and/or for patients who exhibit certain criteria. For example, the patient medical records, for heart failure patients, may be analyzed daily, weekly, monthly and the like.

At 1002, the processor identifies an ST segment in the CA signals for one or more cardiac beats. For example, the processor may analyze the CA signals, within the IMD data, to identify a peak in one or more R waves. When a peak in the R-wave is identified, the processor may set an ST search window to begin a predetermined period of time after the peak of the R-wave (e.g., R-to-ST delay). The ST search window may have a predetermined search length. The processor aligns the ST search window with a portion of the CA signal following the peak of the R-wave by the predetermined period of time (e.g., following the R-to-ST delay). The processor captures the portion of the CA signal that aligns with the ST search window as an ST segment.

At 1004, the processor analyzes the ST segment to determine whether the ST segment has shifted to an elevated level with respect to a prior ST segment level and/or a baseline ST segment level. For example, the processor may identify a peak, average, median, “area under the curve” or other mathematical function indicative of the CA signal during the ST segment to obtain a current ST segment level. The level/change of ST segment elevation may be quantified in volts or millivolts. The processor compares the current ST segment level to one or more prior or baseline ST segment levels. The prior or baseline ST segment levels may be defined in various manners. For example, the processor may maintain a most recent ST segment level as the “prior” ST segment level. Additionally or alternatively, the processor may maintain a running average for a series of prior ST segment levels. Additionally or alternatively, a baseline ST segment level may be programmed by medical personnel at a time of implant of the IMD and/or at various follow-up clinical visits. The baseline ST segment level may be updated remotely, over the medical network, through a local external device in communication with the IMD.

Optionally, the baseline ST segment level may be automatically identified by the IMD. For example, the IMD may determine when a patient is experiencing normal physiologic cardiac beats, from which the IMD identifies the baseline ST segment level. For example, the processor may collect CA signals for the series of normal cardiac beats, identify R-wave peaks therein and overlay the CA signals with an ST search window following the R-wave peak by a predetermine time interval. The processor applies a mathematical function to the CA signals in the ST search window to obtain a current level for the ST segment, and combines one or more levels for one or more corresponding ST segments to form a baseline ST segment level.

At 1006, the processor determines whether the current level for the ST segment has shifted, with respect to a prior or baseline ST segment level, by more than a predetermined threshold. When the shift exceeds a predetermined threshold, flow continues to 1008. When the shift does not exceed the predetermined threshold, flow skips to the end of the operations of FIG. 10A, and no change is made in the patient's health risk index.

At 1008, the processor determines a level of one or more cardiac enzymes indicative of a degree of myocardial damage. For example, the cardiac enzyme may represent troponin I, troponin T and/or complexes of troponin I and T. The BGA device may include an assay system that uses specialized antibodies for the detection and quantitation of troponin I and troponin T in body fluids as an indicator of myocardial infarction. Since troponin I and T exist in various conformations in the blood, the ratios of the monomeric troponin I and T and the binary and ternary complexes, as well as which form of troponin present in the blood, may be analyzed to identify the metabolic state of the heart. As a nonlimiting example, embodiments herein may implement the methods and/or structures described in US patent publication 2012/0129198, titled “NOVEL METHODS FOR THE ESSAY OF TROPONIN I AND T AND COMPLEXES OF TROPONIN I AND T AND SELECTION OF ANTIBODIES FOR USE IN IMMUNOASSAYS”, published May 24, 2012, the complete subject matter which is expressly incorporated herein by reference in its entirety.

At 1010, the processor determines whether the level of the cardiac enzyme (e.g., troponin) exceeds a corresponding threshold. When the cardiac enzyme level exceeds the threshold, the processor interprets this as an indication that a patient is experiencing acute cardiac ischemia, acute myocardial infarction and the like. When the level of the cardiac enzyme does not exceed the corresponding threshold, flow moves to the end of FIG. 10, and no change is made in the patient's health risk index.

Alternatively, at 1010, when the level of the cardiac enzyme exceeds the corresponding threshold, flow moves to 1012. At 1012, the processor assigns a health risk index based on the shift in the ST segment level and the shift in the cardiac enzyme level. Thereafter, the operations of FIG. 10A are completed, and the patient's medical record is updated accordingly with the new ST segment level information, new cardiac enzyme level information, and any changes in the health risk index assigned.

The troponin level may be elevated, alone or in combination with an elevated ST segment level, thereby warranting an assignment of a health risk index indicative of an elevated or severe myocardial ischemia and/or myocardial infarction. The patient may be requested to immediately come to an emergency room or to schedule an appointment in the near future. When an appointment occurs, an additional cardiac enzyme measurement may be obtained to determine a current troponin level or other cardiac enzyme level. The measurements obtained in connection with the patient visit to the emergency room may then be analyzed in combination with one or more prior measurements (e.g., obtained at home).

Optionally, the IMD may monitor CA signals for ST segment elevation. When elevated ST segment level is identified, the IMD may direct an external device to deliver an audible alarm to alert the patient to take a troponin or other cardiac enzyme measurement.

FIG. 10B illustrates a method for utilizing the information collected by the operations of FIG. 10A during or in connection with a patient visit to an emergency room or other medical facility. At 1020, the patient visits an emergency room or other medical facility while experiencing symptoms of interest (e.g., chest pains, shortness of breath). At 1022, a test is performed to obtain new cardiac enzyme levels presently being experienced by the patient. Optionally, the test may collect other BGA data of interest as well. The results of the test are entered into the healthcare system.

After completion of the test at 1022, the operations of FIG. 10B may be implemented in parallel or alternative manners, namely in a generally manual, physician-controlled process and/or an automated matter by the healthcare system. For example, once the newest cardiac enzyme levels (e.g., troponin level) is measured at 1022 and entered into the healthcare system (at 1022), the processor of the healthcare system may implement the operations of FIG. 6D. As an example of an automated implementation, at 1024, a processor of the healthcare system accesses the patient medical record of the current patient to obtain, among other things, previously acquired cardiac enzyme levels and changes, as well as ST segment levels and changes previously measured, such as in connection with. the operations of FIG. 10A. As an example of a manual implementation, medical personnel may access the patient medical record though one or more MP devices (e.g., laptop computer, tablet device, smart phone).

At 1026, a diagnosis is rendered based on the current and prior cardiac enzyme levels, ST segment levels, heart rate and other patient information of interest. The diagnosis at 1026 may be performed automatically by the healthcare system and/or manually by medical personnel. For example, the processor may determine whether the current cardiac enzyme level exceeds a prior cardiac enzyme level by more than a threshold. Optionally, the processor may combine and/or compare the current and prior cardiac enzyme levels in other manners to determine whether a change in the level of the cardiac enzyme is indicative of an acute myocardial ischemia and/or acute myocardial infarction.

Optionally, at 1026, the processor may utilize the IMD data and ST segment level information (determined in connection with FIG. 10A) to further assess whether the patient is experiencing an acute myocardial ischemia and/or acute myocardial infarction.

Optionally, the BRM data may include patient symptoms that are entered through an application on a PDE or other device. Optionally, the patient system may be entered as a voice recording of the patient that the PDE device then passes through an NLP (natural language processing) algorithm to determine keywords that indicate patients state. The symptom related BRM data entered by the patient would represent actual symptoms entered in real-time that would affect the quality of the diagnosis.

The diagnosis at 1026 may be rendered manually by one or more medical personnel and/or automatically by the healthcare system. Optionally, at 1026, in an automated implantation, the processor of the healthcare system may also render a treatment notification based on the cardiac enzyme levels/changes and ST segment levels/changes.

The operations of FIGS. 10A and 10B allow the healthcare system to collect more than one troponin level measurement before rendering an analysis. For example, a prior troponin level measurement is first obtained in connection with the operations of FIG. 10 a, after which a second troponin level measurement may be obtained in connection with a patient visit to an ER (FIG. 10B).

In accordance with the embodiment of FIGS. 10A and 10B, methods and systems are provided herein that enabled transmission and collection of cardiac enzyme levels from an at home point of care system. By maintaining cardiac enzyme levels, and ST segment levels, within a patient's medical record, embodiments herein afford more accurate ER diagnosis of ischemia because this measurement is done early in the symptom of chest pain.

### Application #5—Maintenance of VAD or AF Patients at Desired INR Levels

In accordance with embodiments herein, a healthcare system and method are provided that manage treatment for patients with mechanical circulation support devices, such as maintaining VAD or AF patients at desired INR levels. Among other things, the system and method herein seek to detect when a physiologic COI is within a therapeutic range, such as when a coagulation COI for a patient's blood is within an INR range. The system and method further seek to provide treatment diagnosis and recommendations to maintain the coagulation COI within the therapeutic range. In accordance with aspects herein, at least one goal is to increase a patient's “Time in Therapeutic Range” or TTR. For a given patient, TTR is defined as the duration of time in which the patient's International Normalized Ratio (INR) values were within a desired range.

As one example, when a patient receives a mechanical circulatory support device, such as a VAD or other mechanical IMD, the patient begins taking medication related thereto. In the past, patients have undergone continuous antithrombotic management, including anticoagulation with warfarin, with a targeted international normalized ratio [INR] of 2.0-3.0 and aspirin therapy (81-325 mg daily). Embodiments herein allow frequent measurements of BGA data, through an at home point of care BGA test device. The BGA data is analyzed to identify the patient's INR level and to make adjustments in prescriptions to maintain the INR level at a prescribed level within the therapeutic range and, in some instances, to lower the INR level.

However, an effect of warfarin and similar prescription drugs may vary over time for a given patient based on factors such as a patient's food in-take and other medication. Therefore, the effect that warfarin has on a patient's blood stream will change based on what the patient eats and what other medication the patient takes. Some foods and other drugs may have an unintended influence over an anticoagulation effectiveness of warfarin. Aspects of the medical application #5 seek to control anticoagulants through assessment of certain BGA data to improve the patient's Time in Therapeutic Range, while also accounting for changes in a patient's diet and or other medication.

Patient data, other than coagulation indicative BGA data, is also analyzed to improve an accuracy of the anticoagulant control. For example, the system and method analyze IMD data indicative of AF burden to assist in treatment diagnosis and recommendations, such as whether a coagulation or anti-coagulation prescription dosage change or new dosage is appropriate. The IMD data may include levels/trends in parameters associated with an MCS, pacemaker, cardioverter defibrillator, loop monitor, PAP sensor and the like.

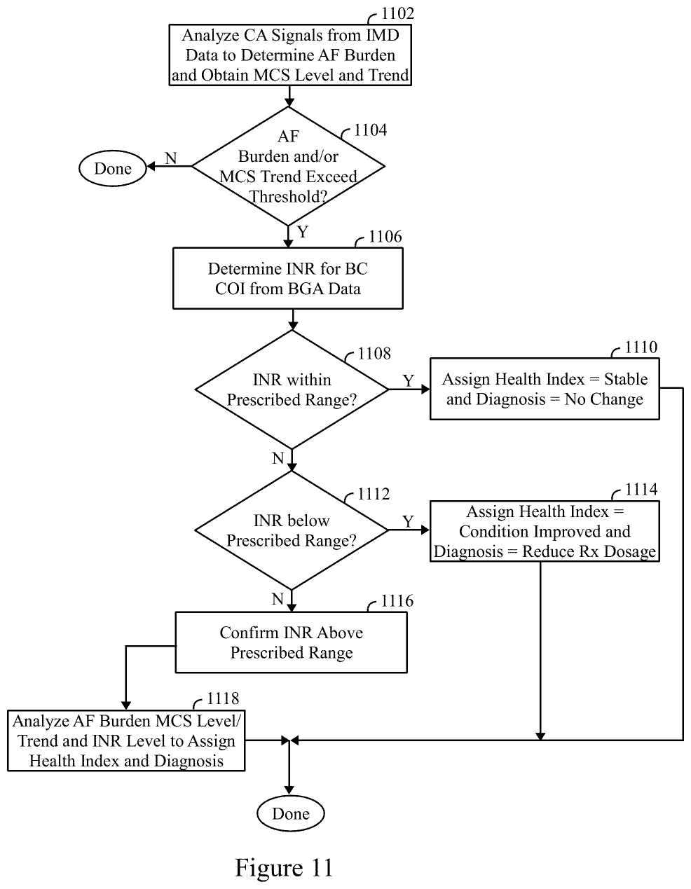

FIG. 11 illustrates a method for managing treatment of patients with a mechanical circulation support device, such as mechanical ventricular assist types of IMDs, in accordance with embodiments herein. The method of FIG. 11 may be initiated based on the various criteria discussed herein. As described in connection with other embodiments, the processor accesses one or more patient medical records. The patient(s) medical record to be analyzed may be identified in various manners. For example, when new IMD data and/or new BGA data is received. For example, the method may be implemented each time a patient collects BGA data indicative of the INR level and/or when new IMD data indicates a start or change in AF burden. Additionally or alternatively, the process may be automatically initiated on a periodic basis for all patients and/or for patients who exhibit certain criteria.

At 1102, the processor of the healthcare system analyzes CA signals from IMD data to determine an AF burden experienced by the patient. In some examples, the analysis of the CA signals to determine the AF burden may be performed in real time in combination with the subsequent operations of FIG. 11. Alternatively, a portion or all of the analysis of the CA signals to determine the AF burden may be performed at a time substantially prior to implementation of the remaining analysis of FIG. 11. When the AF burden is determined at an earlier point in time, the operation at 1102 may simply be to access the AF burden previously recorded. The CA signals from the IMD may indicate that no AF burden is currently being experienced. Optionally, a patient may exhibit no AF burden initially, but after implant of a MCS device (e.g., left ventricular assist device), the patient begins to exhibit a certain level of AF burden. The IMD data collected and analyzed at 1102 may indicate that prior to implant of the LV ND, the patient did not exhibit an AF burden, but did begin to exhibit AF burden after implant. The IMD data may further indicate that onset of the AF burden occurred at a particular point in time with the level of the AF burden leveling off at a certain steady-state, that may then later exhibit an increasing or decreasing trend. Based on the onset, severity and trend in the AF burden, embodiments herein may assign various health indices and generate corresponding diagnoses.

Additionally or alternatively, the processor may analyze PAP signals from a PAP sensor to determine AF episodes and AF burden. For example, embodiments herein may implement one or more of the methods and systems described in one or more of the following:
U.S. Published Application 2013/0204147, titled “ATRIAL FIBRILLATION DETECTION BASED ON PULMONARY ARTERY PRESSURE DATA” and publishing on Aug. 8, 2013, the complete subject matter of which is expressly incorporated herein by reference in its entirety.

Additionally, at 1102, the processor obtains mechanical circulatory support (MCS) data from the IMD data. The CA signals and MCS data may be stored in connection with the patient's medical record and/or directly conveyed from a local external device. The MCS data is indicative of a parameter of an MCS device. For example, the parameter may be indicative of at least one of an RPM level, flow rate or device alert from the MCS device. Additionally or alternatively, the MCS data may represent a raw level and/or trend for one or more parameters, such as an RPM level or trend, flow rate level or trend, change in RPM level, change in flow rate, change in flow level and the like. As a further example, the MCS device may represent a ventricular assist device, where the MCS data is analyzed, as explained herein, in connection with assessing a risk for at least one of hem olysis or thrombosis.

At 1104, the processor determines whether the AF burden and/or MCS level/trend exceeds a threshold. For example, an AF burden threshold and/or MCS threshold may be defined manually by medical personnel or automatically by the healthcare system based on various criteria. As a further example, the AF burden may be defined based on a number of atrial fibrillation episodes experienced within a certain period of time, a number of atrial fibrillation episodes experienced in total, a degree of change in the number of atrial fibrillation episodes experienced and the like. As a further example, the determination at 1104 may include determining whether a number of AF episodes within a recent predetermined period of time (the prior day, the prior week, the prior month) has increased over a prior average number of AF episodes and/or number of AF episodes occurring in prior predetermined periods of time (e.g., daily or weekly). When the AF burden does not exceed the threshold, the process of FIG. 11 may terminate without further analysis. Alternatively, when the AF burden does not exceed the threshold, all or a portion of the remaining operations in FIG. 11 may still be implemented.

Optionally, the MCS threshold may be defined automatically based on prior MCS data acquired from an MCS type IMD. For example, a calibration operation may be implemented to derive baseline information, from which an MCS threshold is calculated. As another example, the MCS threshold may represent an average or other mathematical function characterizing prior samples of MCS data levels over time (e.g., the average RPM level per day, per week). Optionally, the MCS threshold may represent an average or other mathematical function characterizing prior samples of the MCS data during certain cardiac conditions, such as when a patient is experiencing a normal physiologic cardiac signal (e.g., as measured by an implantable pacemaker, cardiac monitor and the like), when a patient is exhibiting a particular heart rate, or steady-state heart rate, a particular blood pressure level and/or steady-state blood pressure, and the like.

At 1106, the processor accesses BGA data recorded in connection with the patient. The BGA data may be previously acquired and stored in the patient's medical record. Optionally, the BGA data may be obtained in real time by a BGA test device during the operations of FIG. 11. At 1106, the processor analyzes a portion of the BGA data indicative of a blood clotting characteristic of interest to derive an INR level associated with the BC COI. By way of example, the BGA data may include coagulation related BGA data, such as levels for ACT Kaolin, ACT Celite and other coagulation related BGA data indicative of a blood clot characteristic of interest. At 1106, the processor also obtains medication information indicating a prescription currently being taken by the patient, such as the level of anticoagulation medication (e.g., warfarin and aspirin).

At 1108, the processor determines whether the INR is within a prescribed range. The prescribed range may be manually set by medical personnel and/or automatically determined and updated by the healthcare system. For example, an individual patient may have an initial INR prescribed range programmed by a physician. The healthcare system may then periodically update the INR range based on additional information collected in connection with the individual patient and/or a patient population. When the newly calculated INR is within the prescribed range, flow moves to 1110. When the newly calculated INR is not within the prescribed range, flow moves to 1112.

At 1110, the processor assigns a healthcare index based on the IMD data (e.g., CA signals, MCS data) and BGA data currently and previously acquired in connection with the patient. For example, the AF burden and/or MCS level/trend may only slightly exceed the threshold at 1104, while the INR remains within the prescribed range, and based thereon the health risk index may be assigned to a stable state and the like. At 1110, the processor may also generate an INR based diagnosis, such as no change. The foregoing example represents a health risk index and diagnosis representative of a relative change in a state of the patient. Additionally or alternatively, health risk index and/or INR based diagnosis may be set to characterize a particular condition of the patient, such as exhibiting medium AF burden, moderate AF burden, low AF burden, exhibiting a low or moderate MCS RPM/flow level and/or exhibiting a steady trend in INR, a decreasing INR trend, an increasing INR trend and the like. Thereafter, flow moves to the end of FIG. 11.

At 1108, when the INR is not within the prescribed range, flow moves to 1112. At 1112, the processor determines whether the INR is below the lower limit of the prescribed range. When the INR is below the prescribed range, the process may interpret the circumstance to indicate that the patient's condition is improving. Accordingly, when the INR is below the prescribed range, flow moves to 1114. Otherwise, flow continues to 1116.

At 1114, the processor assigns a health risk index based on the IMD data and the BGA data currently and previously acquired in connection with the patient. For example, the AF burden and/or MCS level/trend may exhibit a reducing trend (even though the AF burden exceeded a threshold at 1104). Additionally, the INR may exhibit a decreasing trend that has now crossed the lower limit of the prescribed range. In the foregoing example, the health risk index may be assigned to a “condition improved” state and/or other indicator designating that the patient's health condition of interest is improving. At 1114, the processor may also generate an INR based diagnosis, such as to reduce a prescription dosage (e.g., reduce a dosage of anticoagulation prescription such as warfarin). The foregoing example represents a health risk index and diagnosis representative of a relative change in a state of the patient. Additionally or alternatively, the health risk index and/or INR based diagnosis may be assigned to characterize a particular condition of the patient, such as exhibiting decreasing AF burden, and MCS RPM/flow level within a desired range or below a desired threshold, exhibiting a downward INR trend and the like. Thereafter, flow moves to the end of FIG. 11.

When flow advances from 1112 to 1116, the process interprets this condition to indicate that the INR is above the prescribed range. Accordingly, at 1116, the processor confirms that the INR is above the prescribed range. At 1118, the processor assigns a health risk index based on the IMD data and the BGA data currently and previously acquired in connection with the patient. At 1118, the processor may also generate a diagnosis, such as increase a dosage of anticoagulation and the like. For example, the AF burden may exhibit an increasing trend and/or and AF burden level that is substantially above a threshold. Additionally, the INR may exhibit an increasing trend and/or in INR level that is above the upper limit of the prescribed range. Accordingly, the health risk index may be assigned to an “condition deteriorating” state, a severe AF burden state, and excessive INR level state and the like. The foregoing example represents a health risk index and diagnosis representative of a relative change in a state of the patient. Additionally or alternatively, the health risk index and/or INR based diagnosis may be assigned to characterize a particular condition of the patient, such as exhibiting severe AF burden, a high MCS RPM/flow level, a severe INR level and the like. Thereafter, flow moves to the end of FIG. 11.

The operations of FIG. 11 provide, among other things, a way to utilize MCS parameters (e.g., a ventricular assist device RPM level, flow rate, alerts), in combination with AF burden and a BC COI to track a condition of a patient who has received a mechanical circulatory support device. For example, the operations of FIG. 11 may allow for continuous monitoring for future risk of hemolysis or thrombosis. Closer monitoring the foregoing data and parameters, among other things, afford better management of patients with MCS devices.

Optionally, embodiments herein may update the patient medical record to include historic information as appropriate based on patient history at various points throughout the processes described herein.

### Closing Statements

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the Figures, which illustrate example methods, devices and program products according to various example embodiments. These program instructions may be provided to a processor of a general purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A method for managing treatment for a patient, the method comprising:
under control of a processor, obtaining a medical data collection, for the patient, that includes at least two of the following a) to e):
a) implantable medical device (IMD) data indicative of at least one of ai) a hemodynamic COI experienced by the patient, aii) a physiologic COI from the patient, aiii) cardiac activity (CA) signals for one or more cardiac beats; or
b) body generated analyte (BGA) data indicative of at least one of bi) a malnutrition state (MS) COI of a patient, bii) electrolyte COI of a patient, biii) a COI of a heart, iv) a cardiac enzyme COI of a patient;
c) diuretic medication information indicative of a diuretic prescription to a patient;
d) anticoagulant medication information indicative of an anticoagulant prescription to a patient; and
e) behavior related medical (BRM) data indicative of an action, conduct or state by a patient in connection with one or more physiologic COI;
applying an application-specific model (ASM) to the medical data collection to determine a diagnosis and a treatment notification based on the diagnosis;
wherein the ASM is implemented as at least one of a threshold-based algorithm, template correlation algorithm, lookup table, decision tree, or machine learning algorithm, the ASM performing at least one of the following a) to f) determining operations to determine the diagnosis and treatment notification:
f) determining at least one of: f1) whether a patient is a candidate for a procedure, f2) an effectiveness of a prior procedure, f3) a degree of perfusion from pulmonary hypertension experienced by the patient;
g) determining a health risk index indicative of a chronic disease state and malnutrition state currently exhibited by the patient;
h) determining h1) a diuretic response profile of the patient based on the diuretic medication information and hemodynamic data, h2) an heart failure (HF) diagnosis based on the diuretic response profile, the level of the electrolyte COI indicated by the BGA data; and h3) an HF treatment notification based on the HF diagnosis;
i) determining: i1) a risk score for a patient based on the BGA data and the IMD data, the risk score related to a probability that a patient will experience a predetermined event during a predetermined period of time; i2) an HF diagnosis based on the risk score; and d3) an HF treatment notification based on the HF diagnosis;
j) determining: j1) an ST segment level for the one or more cardiac beats based on the CA signals; j2) an myocardial infarction (MI) diagnosis based on the ST segment level and a level of the cardiac enzyme COI indicated by the BGA data; and j3) an MI treatment notification based on the MI diagnosis; or
k) determining: k1) a level for an international normalized ratio (INR) indicative of a blood clotting (BC) characteristic of interest (COI) of a patient based on the BGA data; k2) an INR diagnosis based on the AF burden, the level of the INR and the anticoagulant medication information; and k3) an INR treatment notification based on the INR diagnosis.

2. The method of claim 1, wherein the medical data collection includes the IMD data indicative of the hemodynamic COI experienced by the patient and the ASM determines, as the diagnosis and treatment notification, an indication that a patient is or is not a candidate for at least one of implant of a ventricular assist device, a transplant, or a valve repair procedure.

3. The method of claim 1, wherein the medical data collection includes first and second IMD data from first and second IMDs, respectively, the first IMD including a pulmonary arterial pressure (PAP) sensor, the first IMD data corresponding to hemodynamic data collected by the PAP sensor indicative of ai) the hemodynamic COI, the second IMD connected to two or more subcutaneous electrodes configured to sense CA signals as the second IMD data.

4. The method of claim 3, wherein the ASM analyzes the first and second IMD data, to determine whether the patient is experiencing a select degree of perfusion resulting from pulmonary hypertension.

5. The method of claim 1, wherein the medical data collection includes the IMD data indicative of the hemodynamic COI experienced by the patient, the method further comprising obtaining the IMD data after the patient has undergone a surgical procedure, the treatment notification including an indication regarding an effectiveness of the procedure.

6. The method of claim 1, wherein the medical data collection comprises the BGA data indicative of the MS COI of the patient and further comprising the IMD data indicative of the physiologic COI from the patient, the ASM determining the health risk index by:
calculating a malnutrition state related index (MSI) based on the BGA data, the MSI indicative of a degree of malnutrition experienced by the patient,
calculating a congestion state related index (CSI) based on the IMD data, the CSI indicative of a degree of congestion experienced by the patient; and
generating the diagnosis and treatment recommendation based on the MSI and CSI.

7. The method of claim 6, wherein BGA data includes a serum albumin level of the patient, the ASM calculating a geriatric nutrition risk index (GNRI) level based on the serum albumin level, the ASM classifying the MSI to have one of a number of malnutrition states based on a correlation between the GNRI level and predetermined ranges.

8. The method of claim 1, wherein the medical data collection comprises the BGA data indicative of the level of the electrolyte COI of the patient, the ASM determining the diagnosis and treatment notification, in part, by calculating a level for at least one of glomerular filtration rate (GFR), blood urea nitrogen (BUN) or creatinine from the BGA data.

9. The method of claim 8, wherein the diagnosis represents a HF diagnosis including recommending an increase or decrease in the diuretic prescription based on predetermined combinations of changes in the hemodynamic data and the level of the GFR.

10. The method of claim 8, further comprising identifying cardiorenal syndrome (CRS) based on an increase in the BUN and creatinine, assigning a health risk index of advanced AF, when the CRS is identified in combination with a downward trend in cardiac output and a diuretic resistance indicated by the diuretic response profile.

11. The method of claim 10, wherein at least one of:
i) the BGA data includes malnutrition state BGA (MS-BGA) data indicative of a malnutrition state (MS) COI of the patient, wherein the HF diagnosis declares one of an intravascular volume overload or a total body overload based on the hemodynamic data, diuretic response profile and MS-BGA data;
ii) the BGA data includes a glucose level indicative of a blood sugar level for the patient, the method further comprising identifying episodes of increased pulmonary arterial pressure associated with a decrease in the blood glucose level, and based thereon, generating the HF diagnosis that avoids an increase in a dosage of the diuretic prescription;
iii) the diagnosis includes a recommendation to adjust intake of a nutritional supplement configured to correct a malnutrition state and avoid an increase in a dosage of the diuretic prescription.

12. A system for managing treatment for a patient, the system comprising:
memory configured to store program instructions;
an input configured to obtain a medical data collection, for the patient, that includes at least two of the following a) to e):
a) implantable medical device (IMD) data indicative of at least one of ai) a hemodynamic COI experienced by the patient, aii) a physiologic COI from the patient, aiii) cardiac activity (CA) signals for one or more cardiac beats; or
b) body generated analyte (BGA) data indicative of at least one of bi) a malnutrition state (MS) characteristic of interest (COI) of a patient, bii) an electrolyte COI of a patient, biii) a COI of a heart, iv) a cardiac enzyme COI of a patient;
c) diuretic medication information indicative of a diuretic prescription to a patient;
d) anticoagulant medication information indicative of an anticoagulant prescription to a patient; and
e) behavior related medical (BRM) data indicative of an action, conduct or state by a patient in connection with one or more physiologic COI;
a processor configured to implement the program instructions to:
apply an application-specific model (ASM) to the medical data collection to determine a diagnosis and a treatment notification based on the diagnosis;
wherein the ASM is implemented as at least one of a threshold-based algorithm, template correlation algorithm, lookup table, decision tree, or machine learning algorithm, the ASM configured to perform at least one of the following a) to f) determine operations to determine the diagnosis and treatment notification:
f) determine at least one of: f1) whether a patient is a candidate for a procedure, f2) an effectiveness of a prior procedure, f3) a degree of perfusion from pulmonary hypertension experienced by the patient;
g) determine a health risk index indicative of a chronic disease state and malnutrition state currently exhibited by the patient;
h) determine h1) a diuretic response profile of the patient based on the diuretic medication information and hemodynamic data, h2) an heart failure (HF) diagnosis based on the diuretic response profile, the level of the electrolyte COI indicated by the BGA data; and h3) an HF treatment notification based on the HF diagnosis;
i) determine: i1) a risk score for a patient based on the BGA data and the IMD data, the risk score related to a probability that a patient will experience a predetermined event during a predetermined period of time; i2) an HF diagnosis based on the risk score; and d3) an HF treatment notification based on the HF diagnosis;
j) determine: j1) an ST segment level for the one or more cardiac beats based on the CA signals; j2) an myocardial infarction (MI) diagnosis based on the ST segment level and a level of the cardiac enzyme COI indicated by the BGA data; and j3) an MI treatment notification based on the MI diagnosis; or
k) determine: k1) a level for an international normalized ratio (INR) indicative of a blood clotting (BC) characteristic of interest (COI) of a patient based on the BGA data; k2) an INR diagnosis based on the AF burden, the level of the INR and the anticoagulant medication information; and k3) an INR treatment notification based on the INR diagnosis.

13. The system of claim 12, wherein the medical data collection includes BGA data indicative of the COI of the heart and IMD data indicative of the COI of the heart, the processor is configured to implement the ASM to:
determine a risk score for a patient based on the BGA data and the IMD data, the risk score related to a probability that a patient will experience a predetermined event during a predetermined period of time;
generate, as the diagnosis, an HF diagnosis based on the risk score; and
generate, as the treatment notification, an HF treatment notification based on the HF diagnosis.

14. The system of claim 13, wherein IMD data includes pulmonary arterial pressure (PAP) data, the processor configured to implement the ASM to at least one of:
estimate a PAP probability that the patient will experience the predetermined event during the predetermined period of time based on at least one of a PAP level or PAP trend; or
calculate an overall probability that the patient will experience a heart failure episode, as the predetermined event, the determining the risk score comprising assigning a level to the risk score based on the overall probability.

15. The system of claim 13, wherein the IMD data includes the CA signals for one or more cardiac beats, the processor configured to implement the ASM to:
determine an ST segment level for the one or more cardiac beats based on the CA signals;
generate, as the diagnosis, a myocardial infarction (MI) diagnosis based on the ST segment level and a level of the cardiac enzyme COI indicated by the cardiac enzyme related BGA data; and
generate, as the treatment notification, an MI treatment notification based on the MI diagnosis.

16. The system of claim 15, wherein the processor is further configured to implement the ASM to calculate a troponin level for at least one of troponin I or troponin T from the BGA data, the MI diagnosis generated based on the troponin level.

17. The system of claim 15, wherein the BGA data includes obtaining cardiac enzyme related BGA data by implementing a first test with an at home point-of-care BGA test device to obtain a first troponin level, and by implementing, at a later point in time, a second test with a medical facility BGA test device to obtain a second troponin level, the generating the MI diagnosis based on a relation between the first and second troponin levels.

18. The system of claim 12, wherein the medical data collection includes the IMD data indicative of CA signals for one or more cardiac beats, the BGA data and the anticoagulation medication information indicative of the anticoagulant prescription to the patient, the processor configured to implement the ASM to:
determine an atrial fibrillation (AF) burden based on the CA signals;
determine a level for an international normalized ratio (INR) indicative of a blood clotting (BC) characteristic of interest (COI) of a patient based on the BGA data;
generate an INR diagnosis based on the AF burden, the level of the INR and the anticoagulant medication information; and
generate an INR treatment notification based on the INR diagnosis.

19. The system of claim 18, wherein the IMD data further includes mechanical circulatory support (MCS) data indicative of a parameter of an MCS device, the INR diagnosis generated in part based on the parameter of the MCS device.

20. The system of claim 18, wherein the IMD data includes a parameter that is indicative of at least one of an RPM level, flow rate or device alert from a ventricular assist device (VAD), the processor further configured to implement the ASM to analyze the AF burden and the parameter from the VAD in connection with risk of at least one of hemolysis or thrombosis.
